(19) United States
(12) Patent Application Publication
(10) Pub. No.: US 2008/0267882 A1
(43) Pub. Date: Oct. 30, 2008

(54) Imaging compounds, methods of making imaging compounds, methods of imaging, therapeutic compounds, methods of making therapeutic compounds, and methods of therapy

(75) Inventors: Xiaoyuan Chen, Union city, CA (US); Zibo Li, Logan, UT (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Stanford University, Palo Alto, CA (US)

(21) Appl. No.: 12/150,473

(22) Filed: Apr. 28, 2008

Related U.S. Application Data
(60) Provisional application No. 60/926,816, filed on Apr. 27, 2007.

(51) Int. Cl. A61K 49/04; C07K 5/09; C07K 7/06; C07K 14/00; C07K 5/12; G01N 33/483; A61K 49/00

(52) U.S. Cl. 424/9.4; 530/331; 530/329; 530/328; 530/325; 530/317; 435/40.5; 424/9.1

(57) ABSTRACT

Embodiments of the present disclosure provide for RGD compounds that include a multimeric RGD (arginine-glycine-aspartic acid (Arg-Gly-Asp)) peptide, methods of making the RGD compound, pharmaceutical compositions including RGD compound, methods of using the RGD compositions or the pharmaceutical compositions including RGD compositions, methods of diagnosing and/or targeting angiogenesis related disease and related biological events, kits for diagnosing and/or targeting angiogenesis related disease and related biological events, and the like. In addition, the present disclosure includes compositions used in and methods relating to non-invasive imaging (e.g., positron emission tomography (PET) imaging) of the RGD compounds in vivo.

### CROSS-REFERENCE TO RELATED APPLICATION

This application claims priority to U.S. provisional applications entitled, “IMAGING COMPOUNDS, METHODS OF MAKING IMAGING COMPOUNDS, METHODS OF IMAGING, THERAPEUTIC COMPOUNDS, METHODS OF MAKING THERAPEUTIC COMPOUNDS, AND METHODS OF THERAPY,” having Ser. No. 60/926,816, filed on Apr. 27, 2007, which is entirely incorporated herein by reference.

### STATEMENT REGARDING FEDERALLY SPONSORED RESEARCH OR DEVELOPMENT

This invention was made with government support under Grant No.: 1R01CA119053 awarded by the National Institute of Health (NIH). The government has certain rights in the invention.

### BACKGROUND

Members of the integrin family play vital roles in the regulation of cellular activation, migration, proliferation, survival, and differentiation. Integrin αᵥβ₃ has been found to be highly expressed on osteoclasts and invasive tumors such as late-stage glioblastomas, breast and prostate tumors, malignant melanomas, and ovarian carcinomas. The expression level of integrin αᵥβ₃ is an important factor in determining the invasiveness and metastatic potential of malignant tumors in both experimental tumor models and cancer patients. Therefore, non-invasive imaging of integrin αᵥβ₃ expression using radiolabeled RGD-peptides may provide a unique means of characterizing the biological aggressiveness of a malignant tumor in an individual patient. It should also be noted that integrin is important in other diseases as well.

Cyclic Arginine-Glycine-Aspartic acid (RGD) peptides bind to integrin αᵥβ₃ and can inhibit new blood vessel formation, or angiogenesis. ¹⁸F-labeling of cyclic RGD peptide was first reported by Haubner et al and the tracer ¹⁸F-galacto-RGD exhibited integrin αᵥβ₃ specific tumor uptake in integrin-positive M21 melanoma xenograft model. In the clinical setting, ¹⁸F-galacto-RGD also showed tumor uptake in certain cancer patients yet the SUV values were suboptimal due to the relatively low αᵥβ₃ binding affinity of the monomeric RGD peptide and the imperfect pharmacokinetics. Therefore, we and others have developed a series of dimeric and multimeric RGD peptides to improve the integrin αᵥβ₃ targeting efficacy [7-19]. One tracer in particular, ¹⁸F-fluorobenzoyl-E[c(RGDyK)]₂ (¹⁸F-FB-E[c(RGDyK)]₂, denoted as ¹⁸F-FRGD2, FIG. 1a), exhibited excellent integrin αᵥβ₃-specific tumor imaging with favorable in vivo pharmacokinetics. The binding potential extrapolated from Logan plot graphical analysis of the PET data correlated well with the receptor density measured by SDS-PAGE/autoradiography in various xenograft models. The tumor-to-background ratio at 1 h after injection of ¹⁸F-FRGD2 also gave a good linear relationship with the tumor tissue integrin αᵥβ₃ expression level. However, the overall yield of ¹⁸F-FRGD2 was not satisfactory, due in part, to the bulk of the two cyclic pentapeptides and the prosthetic group N-succinimidyl-4-¹⁸F-fluorobenzoate (¹⁸F-SFB). The glutamate α-amine group has a pKa of 9.47, which is also less reactive than the ε-amino group on the lysine side chain (pKa=8.95) usually used for ¹⁸F-labeling of peptides.

### SUMMARY

Embodiments of the present disclosure provide for RGD compounds that include a multimeric RGD (arginine-glycine-aspartic acid (Arg-Gly-Asp)) peptide, methods of making the RGD compound, pharmaceutical compositions including RGD compound, methods of using the RGD compositions or the pharmaceutical compositions including RGD compositions, methods of diagnosing and/or targeting angiogenesis related disease and related biological events, kits for diagnosing and/or targeting angiogenesis related disease and related biological events, and the like. In addition, the present disclosure includes compositions used in and methods relating to non-invasive imaging (e.g., positron emission tomography (PET) imaging) of the RGD compounds in vivo.

One exemplary embodiment of the present disclosure includes an RGD compound, among others, includes: a multimeric RGD (arginine-glycine-aspartic acid) peptide; a tag, wherein the tag is selected from a detecting unit, a therapeutic unit, or a combination thereof; and a linker connecting the tag and multimeric RGD peptide.

One exemplary embodiment of the present disclosure includes a method of imaging tissue, cells, or a host, among others, includes: contacting with or administering to a tissue, cells, or host a RGD compound, and imaging the tissue, cells, or host with an imaging system.

One exemplary embodiment of the present disclosure includes a method of diagnosing the presence of one or more angiogenesis related diseases or related biological events in the tissue, cells, or a host, among others, includes: contacting or administering to a tissue, cells, or a host an RGD compound; and imaging the tissue, cells, or a host with an imaging system, wherein the location of the RGD compound corresponds to the location of the angiogenesis related diseases or related biological events.

These embodiments, uses of these embodiments, and other uses, features and advantages of the present disclosure, will become more apparent to those of ordinary skill in the relevant art when the following detailed description of the preferred embodiments is read in conjunction with the appended figures.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIGS. 1-1a to 1-1d illustrate embodiments of RGD compounds.

FIGS. 1-2a to 1-2d illustrate embodiments of multimer RGD peptides.

FIGS. 1-3a and 1-3b illustrate embodiments of tags.

FIGS. 1-4a and 1-4b illustrate embodiments of linkers.

FIGS. 1-5a to 1-5e illustrate embodiments of RGD compounds.

FIG. 1-6a illustrates a method of making RGD compounds.

FIG. 1-6b illustrates an embodiment of a RGD compound made using the method shown in FIG. 1-6a

FIG. 2-1 illustrates the chemical structures of ¹⁸F-FRGD2 (a) and ¹⁸F-FPRGD2 (b). The only difference between the two structures is the mini-PEG spacer.

FIG. 2-2(a) illustrates serial microPET images of U87MG tumor-bearing mice after intravenous injection of ¹⁸F-FPRGD2. FIG. 2-2(b) illustrates, for direct visual comparison, serial microPET images of U87MG tumor-bearing mice after intravenous injection of ¹⁸F-FRGD2 are also shown. FIG. 2-2(c) illustrates the coronal and sagittal microPET images of a U87MG tumor-bearing mouse 1 h after co-injection of ¹⁸F-FPRGD2 and a blocking dose of c(RGDyK). Note that the scale (0-2.5% ID/g) is different from those in (a) and (b) (0-5% ID/g). FIG. 2-2(d) illustrates microPET images of a c-neu oncomouse after intravenous injection of ¹⁸F-FPRGD2. Arrows indicate tumors in all cases.

FIG. 2-3 illustrates the time-activity curves of major organs after intravenous injection of ¹⁸F-FPRGD2.

FIG. 2-4 illustrates a comparison between ¹⁸F-FRGD2 and ¹⁸F-FPRGD2 in U87MG tumor, kidneys, liver, and muscle over time.

FIG. 2-5 illustrates the metabolic stability of ¹⁸F-FPRGD2 in mouse blood and urine samples and in liver, kidneys, and U87MG tumor homogenates at 1 h after injection. The HPLC profile of pure ¹⁸F-FPRGD2 (Standard) is also shown.

FIG. 3-1(A) illustrates a radiosynthesis of scheme of ¹⁸F-FPRGD4. FIG. 3-1(B) illustrates a chemical structure of ¹⁸F-FPRGD4.

FIG. 3-2(A) illustrates a decay-corrected whole-body coronal microPET images of athymic female nude mice bearing U87 MG tumor at 5, 15, 30, 60, 120 and 180 min post-injection (p.i.) of ¹⁸F-FPRGD4 (3.7 MBq [100 μCi]). FIG. 3-2(B) illustrates the decay-corrected whole-body coronal microPET images of c-neu oncomice at 30, 60 and 150 min (5-min static image) after intravenous injection of ¹⁸F-FPRGD4. FIG. 3-2(C) illustrates the decay-corrected whole-body coronal microPET images of orthotopic MDA-MB-435 tumor-bearing mouse at 30, 60 and 150 min after intravenous injection of ¹⁸F-FPRGD4. FIG. 3-2(D) illustrates the decay-corrected whole-body coronal microPET images of DU-145 tumor-bearing mouse (5-min static image) after intravenous injection of ¹⁸F-FPRGD4. FIG. 3-2(E) illustrates the coronal microPET images of a U87 MG tumor-bearing mouse at 30 min and 60 min after co-injection of ¹⁸F-FPRGD4 and a blocking dose of c(RGDyK). Arrows indicate tumors in all cases.

FIG. 3-3 illustrates the time-activity curves of major organs after intravenous injection of ¹⁸F-FPRGD4. Data was derived from multiple time-point microPET study. ROIs are shown as the % ID/g±SD (n=3).

FIG. 3-4 illustrates a comparison between the uptake of ¹⁸F-FRGD2 and ¹⁸F-FPRGD4 in U87 MG tumor, kidneys, liver, and muscle over time. Data was derived from multiple time-point microPET study. ROIs are shown as the % ID/g±SD (n=3).

FIG. 3-5 illustrates the immunofluorescent staining of 3 and CD31 for tumor, liver, kidney and lung. For β₃ staining, frozen tissue slices (5 μm thick) were staining with a hamster anti mouse β₃ primary antibody and a cy3-conjugated goat anti-hamster secondary antibody. For CD31 staining, frozen tissue slices were stained with a rat antimouse CD31 primary antibody and a FITC-conjugated goat anti-rat secondary antibody. (total magnification: 200×).

FIG. 3-6 illustrates the inhibition of ¹²⁵I-echistatin (integrin αᵥβ₃ specific) binding to αᵥβ₃ integrin on U87 MG cells by RGD4, PRGD4 and FPRGD4.

FIG. 3-7(A) illustrate the comparison between the uptakes of ¹⁸F-FPRGD4 in different tumors and kidneys over time for tumor-bearing mice. Data was derived from multiple time-point microPET study. ROIs are shown as the % ID/g±SD (n=3). FIG. 3-8(B) illustrates the direct visual comparison of microPET images of U87MG tumor-bearing mice after intravenous injection of ¹⁸F-FPRGD4 and ¹⁸F-FPRGD2. FIG. 3-8(C) illustrates a comparison of biodistribution (based on PET, 60 min p.i.) results for ¹⁵F-FPRGD4 and ¹⁸F-FPRGD2 on U87MG tumor-bearing mice.

FIG. 3-8 illustrates the immunofluorescent staining of integrin β₃ and CD31 for tumor, liver, kidney, and lung of athymic nude mice. For β₃ staining, frozen tissue slices (5-μm thick) were stained with a hamster antimouse β₃ primary antibody and a Cy3-conjugated goat antihamster secondary antibody. For CD31 staining, frozen tissue slices were stained with a rat antimouse CD31 primary antibody and a FITC-conjugated goat antirat secondary antibody (×200).

FIG. 4-1(A) illustrates the radiosynthesis of ¹⁸F-fluoro-PEG-alkyne intermediate and 1.3-dipolar cycloaddition with terminal azide. R=targeting biomolecule (peptides, proteins, antibodies et al.). FIG. 4-1(B) illustrates a structure of ¹⁸F-fluoro-PEG-alkyne labeled E[c(RGDyK)]₂: ¹⁸F-fluoro-PEG-triazole-E(RGDyK)₂ (¹⁸F-FPTA-RGD2).

FIG. 4-2 illustrates a cell binding assay of E[c(RGDyK)]₂ and FPTA-RGD2 using U87MG cells with competitive displacement studies using ¹²⁵I-echistatin. The IC₅₀ values for E[c(RGDyK)]₂ and FPTA-RGD2 were 79.2±4.2 and 144±6.5 nM, respectively (n=3).

FIG. 4-3(A) illustrates a decay-corrected whole-body coronal microPET images of athymic female nude mice bearing U87MG tumor at 10, 20, 30, 60 and 125 min post-injection (p.i.) of about 2 MBq of ¹⁸F-FPTA-RGD2. FIG. 4-3(B) illustrates the coronal microPET images of U87MG tumor-bearing mice at 30 and 60 min p.i. of ¹⁸F-FPTA-RGD2 with (denoted as “Blocking”) and without coinjection of 10 mg/kg mouse body weight of c(RGDyK). Tumors are indicated by arrows.

FIG. 4-4 illustrates the time-activity curves of the U87MG tumor, liver, kidney, blood, and muscle after intravenous injection of ¹⁸F-FPTA-RGD2. Data were derived from multiple time-point microPET study. ROIs are shown as the % ID/g±SD (n=3). Note that the kidney uptake in the figure is ¼ of the actual value.

FIG. 4-5 illustrates a comparison of ¹⁸F-FPTA-RGD2, ¹⁸F-FB-RGD2 (¹⁸F-FRGD2) and ¹⁸F-FB-PEG3-RGD2 (¹⁸F-FPRGD2) in U87MG tumor, kidney, liver, muscle, and blood over time.

FIG. 4-6 illustrates a metabolic stability of ¹⁸F-FPTA-RGD2 in mouse blood and urine samples and in liver, kidney and U87MG tumor homogenates at 1 h after injection. The HPLC profile of pure ¹⁸F-FPTA-RGD2 (Standard) is also shown.

FIG. 5-1 illustrates chemical structures of DOTA-RGD tetramer (A) and DOTA-RGD octamer (B).

FIGS. 5-2(A) to 5-2(C) illustrate an in vitro cell adhesion assay and cell binding assay using U87MG human glioblastoma cells. FIG. 5-2(A) illustrates a cell adhesion assay of RGD monomer, dimer, tetramer and octamer on fibronectin coated plates (n=4, mean±SD). FIG. 5-2(B) illustrates a cell adhesion assay of RGD monomer, dimer, tetramer and octamer on vitronectin coated plates (n=4, mean±SD). FIG. 5-2(C) illustrates Inhibition of ¹²⁵I-echistatin (integrin αᵥβ₃ specific) binding to αᵥβ₃ integrin on U87MG cells by RGD dimer, tetramer, octamer, DOTA-RGD tetramer, and DOTA-RGD octamer (n=3, mean±SD).

FIGS. 5-3(A)-(C) illustrate microPET studies of U87MG tumor-bearing mice and c-neu oncomice. FIG. 5-3(A) illustrates a decay-corrected whole-body coronal microPET images of athymic female nude mice bearing U87MG tumor at 30 min, 1, 2, 6, and 20 h post-injection (p.i.) of about 9 MBq of ⁶⁴Cu-DOTA-RGD tetramer or ⁶⁴Cu-DOTA-RGD octamer. FIG. 5-3(B) illustrates a coronal microPET images of U87MG tumor-bearing mice at 2 h p.i. of ⁶⁴Cu-DOTA-RGD tetramer or ⁶⁴Cu-DOTA-RGD octamer without and with (denoted as “Blocking”) coinjection of 10 mg/kg mouse body weight of c(RGDyK). FIG. 5-3(C) illustrates a decay-corrected whole-body coronal microPET images of c-neu oncomice at 1, 5, and 20 h p.i. of about 9 MBq of ⁶⁴Cu-DOTA-RGD tetramer or ⁶⁴Cu-DOTA-RGD octamer. These mice are 7 months old and all of them have multiple tumors. ⁶⁴Cu-DOTA-RGD tetramer and ⁶⁴Cu-DOTA-RGD octamer are abbreviated in the figure as “RGD tetramer” and “RGD octamer”, respectively. All images shown are of 5 or 10 min static scans and representative of 3 mice per group. Tumors are indicated by arrows.

FIGS. 5-4(A) and (B) illustrate a quantitative analyses of the microPET data. FIG. 5-4(A) illustrates a comparison between ⁶⁴Cu-DOTA-RGD tetramer and ⁶⁴Cu-DOTA-RGD octamer uptake in the U87MG tumor, liver, kidneys, and muscle over time in the U87MG xenograft model (n=3). FIG. 5-4(B) illustrates a comparison between ⁶⁴Cu-DOTA-RGD tetramer and ⁶⁴Cu-DOTA-RGD octamer uptake in the spontaneous tumor, liver, kidney, and muscle over time in the c-neu oncomice (n=3).

FIGS. 5-5(A)-(D) illustrates a biodistribution and receptor blocking experiments. FIG. 5-5(A) illustrates a biodistribution of ⁶⁴Cu-DOTA-RGD tetramer and ⁶⁴Cu-DOTA-RGD octamer in female athymic nude mice at 20 h post-injection (p.i.) (n=3). Note that the kidney uptake of ⁶⁴Cu-DOTA-RGD octamer plotted in the figure is ⅕ of the actual value (*). FIG. 5-5(B) illustrates a biodistribution of ⁶⁴Cu-DOTA-RGD tetramer in female athymic nude mice at 20 h p.i. with and without coinjection of 10 mg/kg of c(RGDyK) (n=3). FIG. 5-5(C) illustrates a comparison of ⁶⁴Cu-DOTA-RGD tetramer uptake at 2 h p.i. in the U87MG tumor, kidneys, liver, and muscle over time with and without coinjection of 10 mg/kg c(RGDyK) (n=3). FIG. 5-5(D) illustrates a comparison of ⁶⁴Cu-DOTA-RGD octamer uptake in the U87MG tumor, kidneys, liver, and muscle over time with and without coinjection of 10 mg/kg c(RGDyK) (n=3). Note that the kidney uptake of ⁶⁴Cu-DOTA-RGD octamer plotted in the figure is ⅕ of the actual value (*).

FIG. 6-1 illustrates the chemical structure of NOTA-RGD1, NOTA-RGD2 and NOTA-RGD4.

FIG. 6-2 illustrates an inhibition of ¹²⁵I-echistatin (integrin αᵥβ₃-specific) binding to integrin αᵥβ₃ on U87MG cells by NOTA-RGD1, NOTA-RGD2 and NOTA-RGD4 (n=3, mean±SD).

FIG. 6-3(A) illustrates a decay-corrected whole-body coronal microPET images of athymic male nude mice bearing U87MG tumor from 1 h dynamic scan and a static scan at 2 h time point after injection of ⁶⁸Ga-NOTA-RGD1 , ⁶⁸Ga-NOTA-RGD2 and ⁶⁸Ga-NOTA-RGD4 (3.7 MBq/mouse). Tumors are indicated by arrows. FIG. 6-3(B) illustrates a time-activity curves of tumor and major organs after intravenous injection of ⁶⁸Ga-NOTA-RGD1, ⁶⁸Ga-NOTA-RGD2 and ⁶⁸Ga-NOTA-RGD4.

FIG. 6-4(A) illustrates a decay-corrected whole-body coronal microPET images of athymic male nude mice bearing U87MG tumor from static scan at 30, 60 and 120 min time point after injection of ⁶⁸Ga-NOTA-RGD1, ⁶⁸Ga-NOTA-RGD2 and ⁶⁸Ga-NOTA-RGD4 (3.7 MBq/mouse) (n=3 per tracer). Tumors are indicated by arrows. FIG. 6-4(B) illustrates a decay-corrected whole-body coronal microPET images of U87MG tumor bearing mice at 1 h after injection of ⁶⁸Ga-NOTA-RGD2 with/without a blocking dose of c(RGDyK) (10 mg/kg). Tumors are indicated by arrows. FIG. 6-4(C) illustrates time-activity curves of tumor and major organs after intravenous injection of ⁶⁸Ga-NOTA-RGD1, ⁶⁸Ga-NOTA-RGD2 and ⁶⁸Ga-NOTA-RGD4. FIG. 6-4(D) illustrates a comparison of tumor-to-normal organ/tissue (muscle, kidney, liver) ratios of ⁶⁸Ga-NOTA-RGD1, ⁶⁸Ga-NOTA-RGD2 and ⁶⁸Ga-NOTA-RGD4. FIG. 6-4(E) illustrates a comparing the uptake of ⁶⁸Ga-NOTA-RGD2 in U87MG tumor and major organs with/without preinjection of blocking dose of c(RGDyK) peptide (10 mg/kg). Regions of interest (ROIs) are shown as percent injected dose per gram tissue (% ID/g)±SD (n=3).

FIG. 6-5 illustrates the biodistributions of ⁶⁸Ga-NOTA-RGD2 in U87MG tumor-bearing athymic nude mice at 1 h with and without coinjection of 10 mg/kg of c(RGDyK) as a blocking agent. Data are expressed as normalized accumulation of activity in % ID/g±SD (n=3).

FIG. 7-1 illustrates chemical structures of PTX and RGD2−PTX conjugate.

FIG. 7-2(A) illustrates the effect of solvent only, RGD2+PTX and RGD2−PTX treatment on the growth of MDA-MB-435 breast cancer model. Averaged tumor size was monitored every three days and shown as mean±SE (n=8/group). FIG. 7-2(B) illustrates the mice weight of control group or treatment group over time (n=8/group). The drug administration intervals were indicated by arrows. Where * or # denotes P<0.05, ** denotes P<0.01. * and **, compared with solvent control group, # compared with RGD2+PTX treatment group.

FIG. 7-3(A) illustrates representative whole-body coronal microPET images of MDA-MB-435 tumor bearing mice with ¹⁸F-FDG at day 10 during the therapy. FIG. 7-3(B) illustrates comparison between the uptake of ¹⁸F-FDG in MDA-MB-435 tumor with solvent treatment only, RGD2+PTX or RGD2−PTX. Regions of interest (ROIs) were shown as % ID/g±SD (n=3/group). FIG. 7-3(C) illustrates representative whole-body coronal microPET images of MDA-MB-435 tumors bearing mice with ¹⁸F-FLT at day 11 during the therapy. FIG. 7-3(D) illustrates comparison between the uptake of ¹⁸F-FLT in MDA-MB-435 tumors with solvent treatment only, RGD2+PTX or RGD2−PTX. Regions of interest (ROIs) were shown as % ID/g±SD (n=3/group). Tumors were indicated by arrows. Where * denotes P<0.05, ** denotes P<0.01.

FIG. 7-4 illustrates immunofluorescence staining of DAPI, human integrin αᵥβ₃, TUNEL and the overlay for MDA-MB-435 tumor tissue from three treatment groups.

FIG. 7-5(A) illustrates immunofluorescence staining of DAPI, CD31, and the overlay for MDA-MB-435 tumor tissues from three treatment groups. FIG. 7-5(B) illustrates microvessel density (MVD) analysis of MDA-MB-435 tumor tissues from three treatment groups (n=10/group). Where ** or ## denotes P<0.01, *** denotes P<0.01. ** and ***, compared with solvent control group, ## compared with RGD2+PTX treatment group.

FIG. 7-6(A) illustrates the immunofluorescence staining of Ki67, DAPI, and the overlay for MDA-MB-435 tumor tissues from the control, RGD2+PTX, and RGD2−PTX treatment groups. FIG. 7-6(B) illustrates Ki67 positive cell counting showed little or no difference among three treatment groups (P>0.05).

FIG. 8-1 illustrates microPET images of rat myocardial infarction with 18F-FPRGD2. Transaxial images of the same animal on day 7 and 13 were shown. Both wound and the iinfarcted myocardium showed positive signal.

FIG. 8-2 illustrates microPET images of rat myocardial infarction with 64Cu-DOTA-RGD tetramer and FDG. In particular, the representative images are the following: ⁶⁴Cu-DOTA-RGD tetramer (left), ¹⁸F-FDG (right), and ⁶⁴Cu-DOTA-RGD tetramer-¹⁸F-FDG fused image (middle). FDG scan shows that coronary artery ligation resulted in a lack of ¹⁸F-FDG uptake, and that the uptake of ⁶⁴Cu-DOTA-RGD tetramer occurs in areas supplied by the ligated coronary artery. Fusion of both scans results in complementation of ¹⁸F-FDG and ⁶⁴Cu-DOTA-RGD tetramer signals. There is also increased uptake in the area of the surgical wound.

FIG. 9-1 illustrates representative coronal images of microPET scans of stroke rats at day 1 and day 9 after a suture model produced by permanent occlusion of the distal middle cerebral artery (dMCAO). Both wound and the lesion were detectable at day 1. At day 9, the wound signal is significantly decreased, but the signal in the lesion reflecting angiogenesis is remained.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

In describing and claiming the disclosed subject matter, the following terminology will be used in accordance with the definitions set forth below.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gin, His), (Asp: Glu, Cys, Ser), (Gin: Asn), (Glu: Asp), (Gly: Ala), (His: Asn, Gln), (lie: Leu, Val), (Leu: lie, Val), (Lys: Arg), (Met: Leu, Tyr), (Ser: Thr), (Thr: Ser), (Tip: Tyr), (Tyr: Trp, Phe), and (Val: lie, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxyethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

As used herein, the term “imaging probe”, “imaging agent”, or “imaging compound” refers to the labeled compounds of the present disclosure that are capable of serving as imaging agents and whose uptake is related to the expression level of certain surface cell receptors (e.g., integrin αᵥβ₃). In particular non-limiting embodiments the imaging probes or imaging agents of the present disclosure are labeled with a PET isotope, such as F-18, Cu-64, and Ga-68.

By “administration” is meant introducing a compound of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “a detectably effective amount” of the imaging agent of the present disclosure is defined as an amount sufficient to yield an acceptable image using equipment that is available for clinical use. A detectably effective amount of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The term “therapeutically effective amount” as used herein refers to that amount of the compound being administered which will relieve to some extent one or more of the symptoms of a disease, a condition, or a disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

“Treating” or “treatment” of a disease (or a condition or a disorder) includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms also mean that the life expectancy of an individual affected with a cancer will be increased or that one or more of the symptoms of the disease will be reduced.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to host or organisms noted above that are alive and are not dead. The term “living host” refers to the entire host or organism and not just a part excised (e.g., a liver or other organ) from the living host.

The term “sample” can refer to a tissue sample, cell sample, a fluid sample, and the like. The sample may be taken from a host. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “detectable signal” is a signal derived from non-invasive imaging techniques such as, but not limited to, positron emission tomography (PET), single photon emission computed tomography (SPECT), optical imaging, magnetic resonance imaging (MRI), computer topography (CT), or ultrasound. The detectable signal is detectable and distinguishable from other background signals that may be generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the acoustic detectable signal and/or the background.

Angiogenesis is the physiological process involving the growth of new blood vessels. Excessive angiogenesis occurs when diseased cells produce abnormal amounts of angiogenic growth factors, overwhelming the effects of natural angiogenesis inhibitors. Imbalances between the production of angiogenic growth factors and angiogenesis inhibitors can cause improperly regulated growth or suppression of vascular vessels. Angiogenesis-dependent or related diseases result when new blood vessels either grow excessively or insufficiently. The angiogenesis related disease can include diseases such as, but not limited to, cancer, precancerous tissue, tumors, cardiac infarction, and stroke. Excessive angiogenesis can include: cancer, diabetic blindness, age-related macular degeneration, rheumatoid arthritis, psoriasis, and more than 70 other conditions. Insufficient angiogenesis can include: coronary artery disease, stroke, and delayed wound healing. In particular, angiogenesis related disease includes diseases and conditions including or relating to the vitronectic receptor integrin αᵥβ₃ Additional details regarding integrin αᵥβ₃ are described in the Examples.

“Cancer”, as used herein, shall be given its ordinary meaning, as a general term for diseases in which abnormal cells divide without control. In particular, cancer refers to angiogenesis related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

It should be noted that precancerous cells, cancer, and tumors are often used interchangeably in the disclosure.

Diseases with ischemic or hypoxic mechanisms (e.g., ischemic or hypoxic related diseases) can be subclassified into general diseases and cerebral ischemia. Examples of such general diseases involving ischemic or hypoxic mechanisms include myocardial infarction, cardiac insufficiency, cardiac failure, congestive heart failure, myocarditis, pericarditis, perimyocarditis, coronary heart disease (stenosis of coronary arteries), angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema. Examples of cerebral ischemia disease include stroke (as well as hemorrhagic stroke), cerebral microangiopathy (small vessel disease), intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy.

### General Discussion

The present disclosure provides for RGD compounds that include a multimeric RGD (arginine-glycine-aspartic acid (Arg-Gly-Asp)) peptide, methods of making the RGD compound, pharmaceutical compositions including the RGD compound, methods of using the RGD compositions or the pharmaceutical compositions including RGD compositions, methods of diagnosing and/or targeting angiogenesis related disease and related biological events, kits for diagnosing and/or targeting angiogenesis related disease and related biological events, and the like. In addition, the present disclosure includes compositions used in and methods relating to non-invasive imaging (e.g., positron emission tomography (PET) imaging) of the RGD compounds in vivo.

Embodiments of the present disclosure include methods for imaging tissue, cells, or a host that includes contacting with or administering to a tissue, cells, or host, an RGD compound, and imaging the tissue with a PET imaging system. The imaging can be performed in vivo and/or in vitro. In particular, embodiments of the present disclosure can be used to image angiogenesis related diseases or related biological events. In this regard, the tissue, cells, or host can be tested to determine if the tissue, cells, or host include angiogenesis related diseases or related biological events. The tissue can be within a host or have been removed from a host.

In addition, embodiments of the present disclosure include methods of monitoring the progress of one or more angiogenesis related diseases or related biological events in the tissue, cells, or a host, by contacting or administering to a tissue with, an RGD compound and imaging the tissue with a PET imaging system.

Another embodiment of the present disclosure includes pharmaceutical compositions for imaging angiogenesis related diseases or related biological events that include an RGD compound.

Embodiments of the present disclosure provide RGD compounds that include a multimeric RGD peptide that can be made for cell adhesion molecule integrin αvβ3 targeting with high affinity and specificity based upon the “polyvalency effect”. The resulting RGD compounds are superior to literature reported integrin ligands in terms of imaging quality (when coupled with an imaging tag) and therapeutic efficacy (when coupled with cytotoxic compound or therapeutic radioisotope).

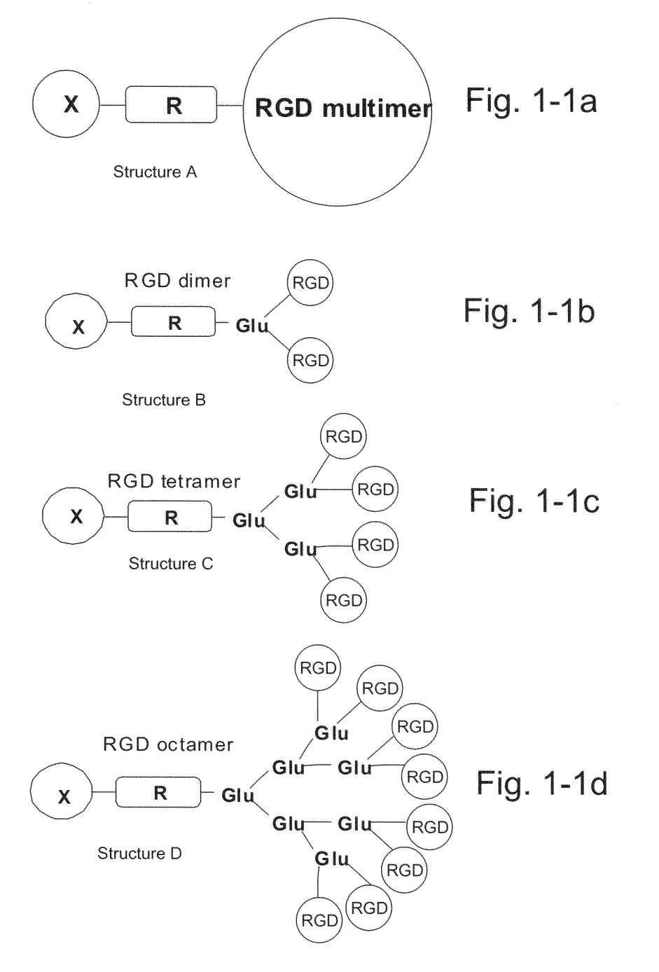

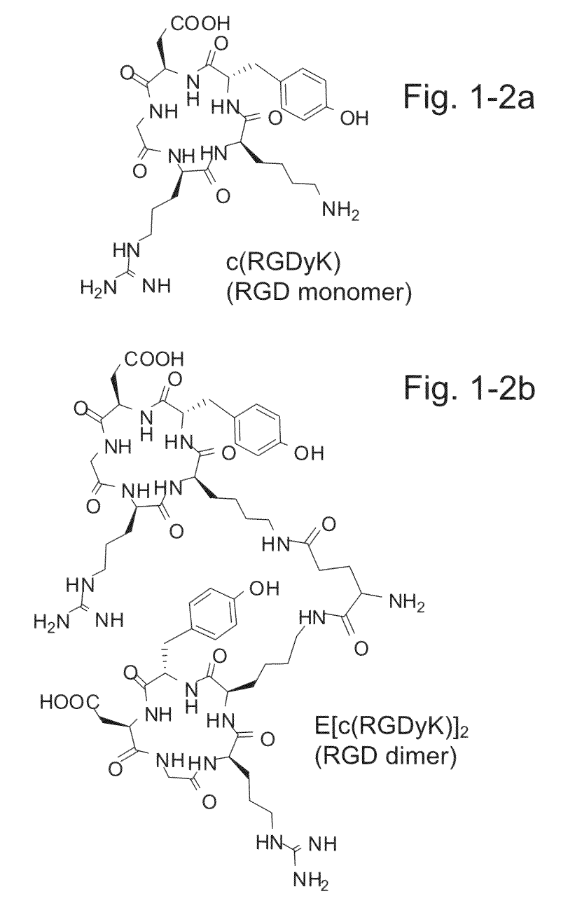

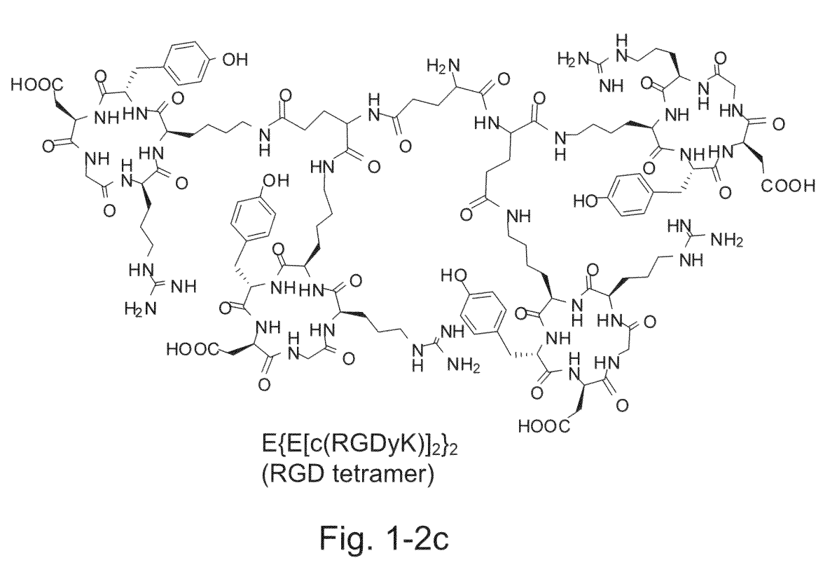

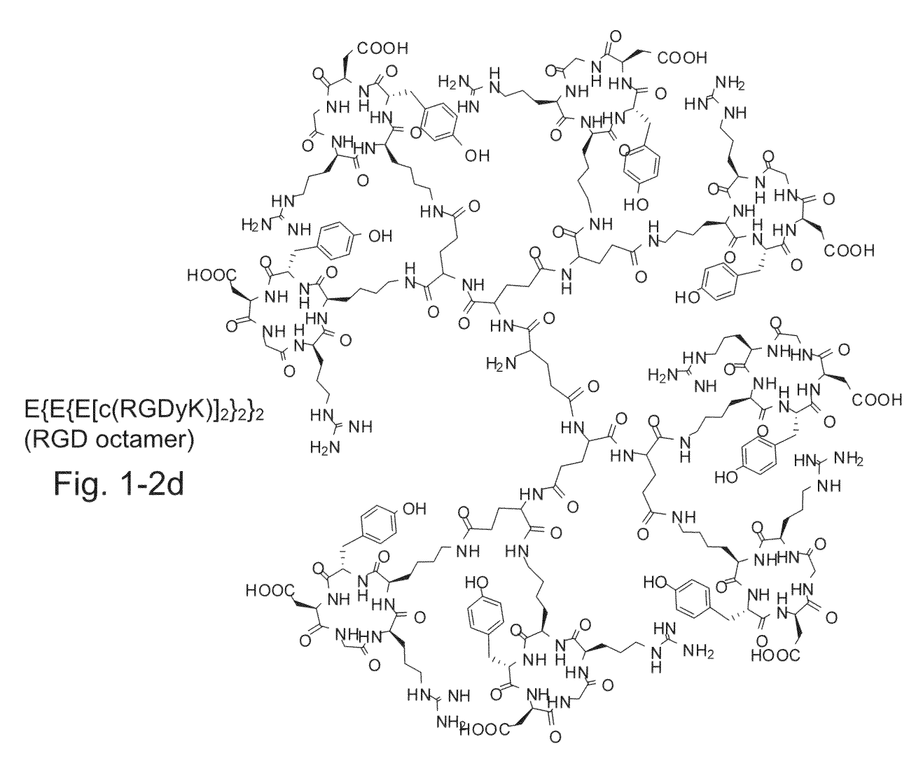

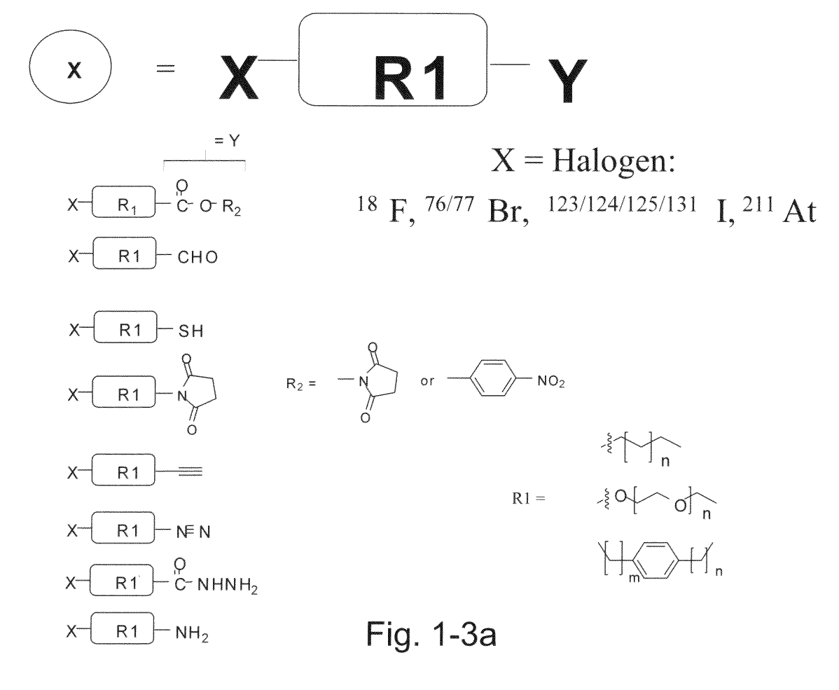

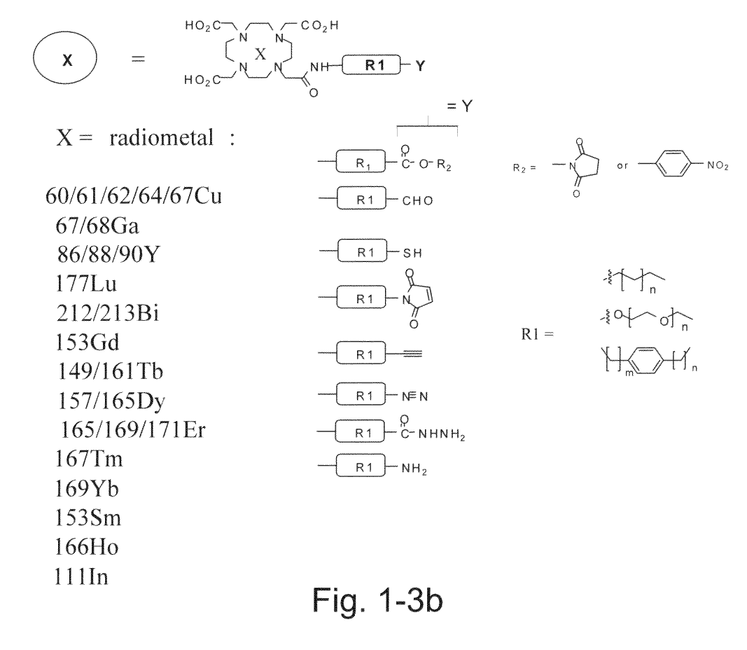

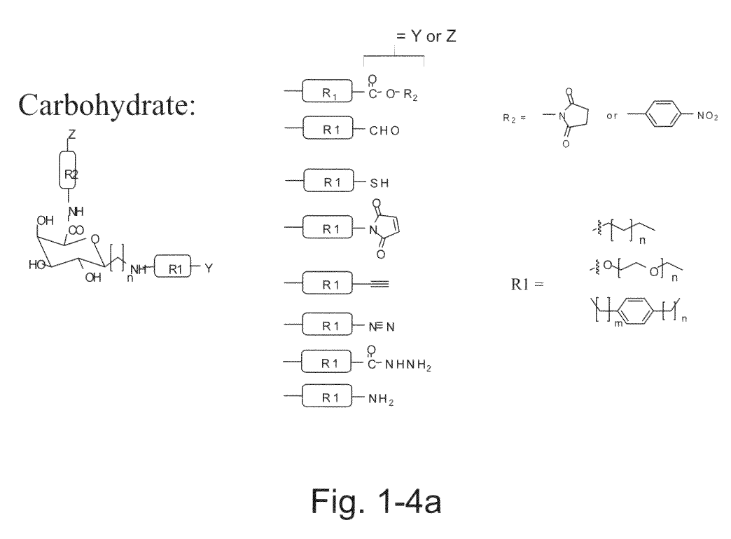

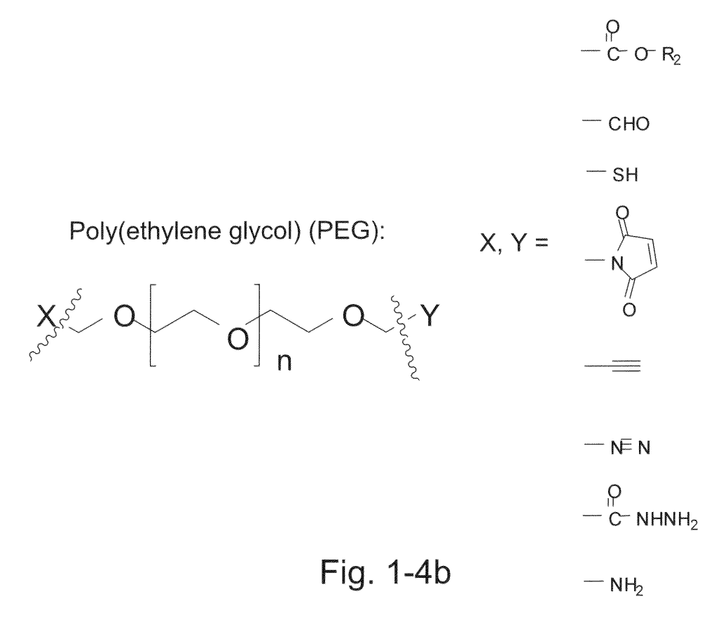

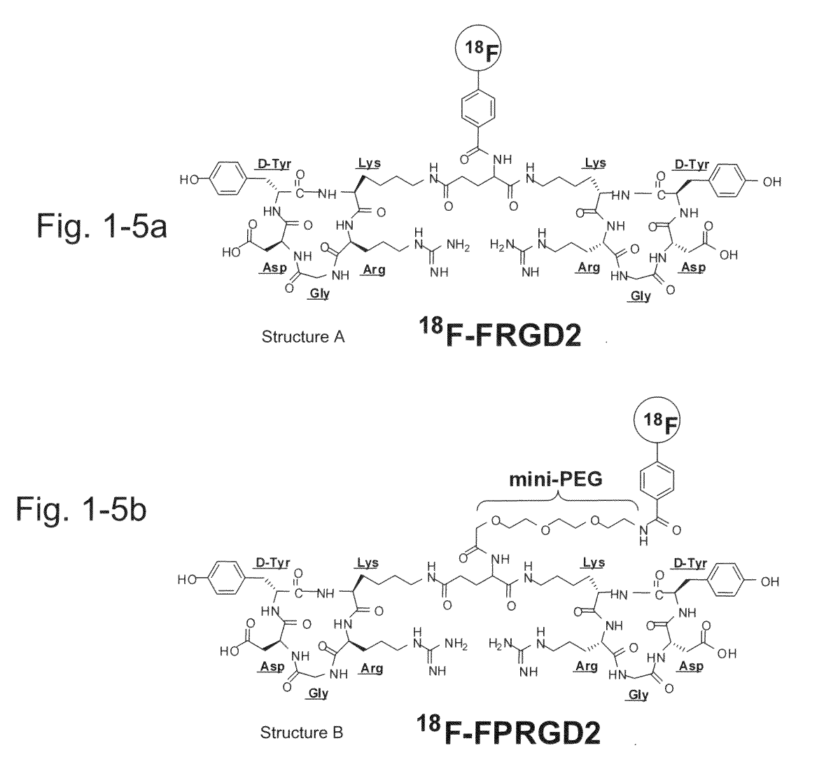

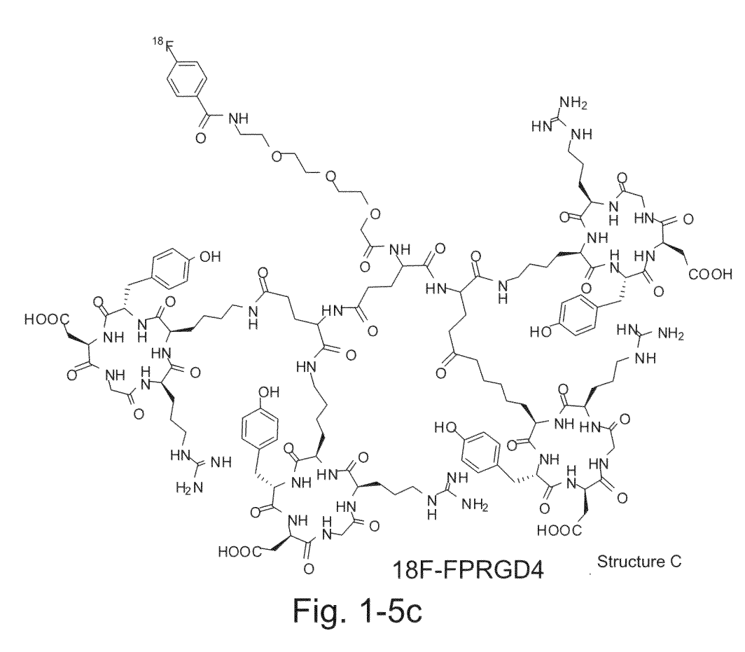

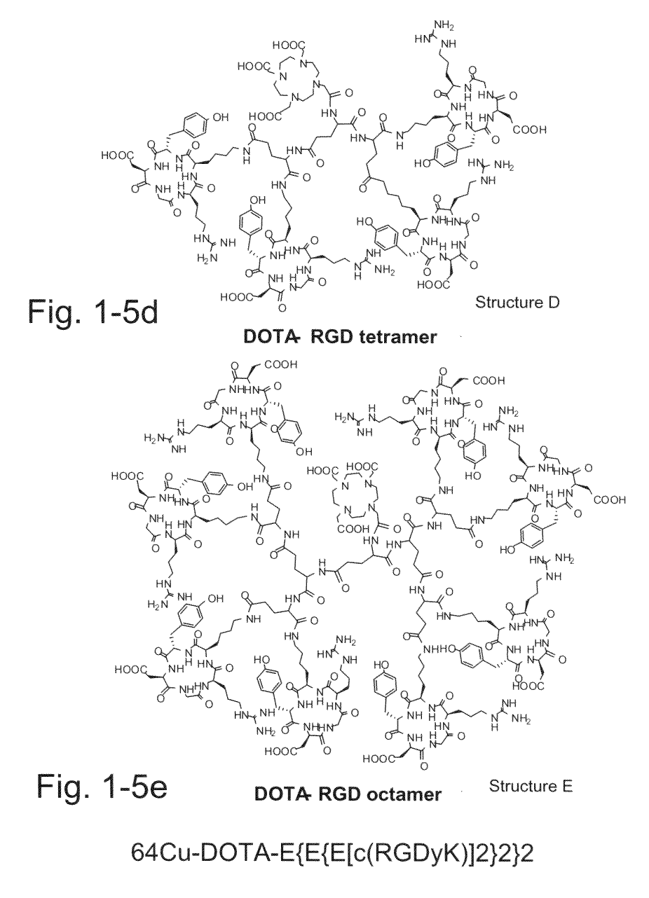

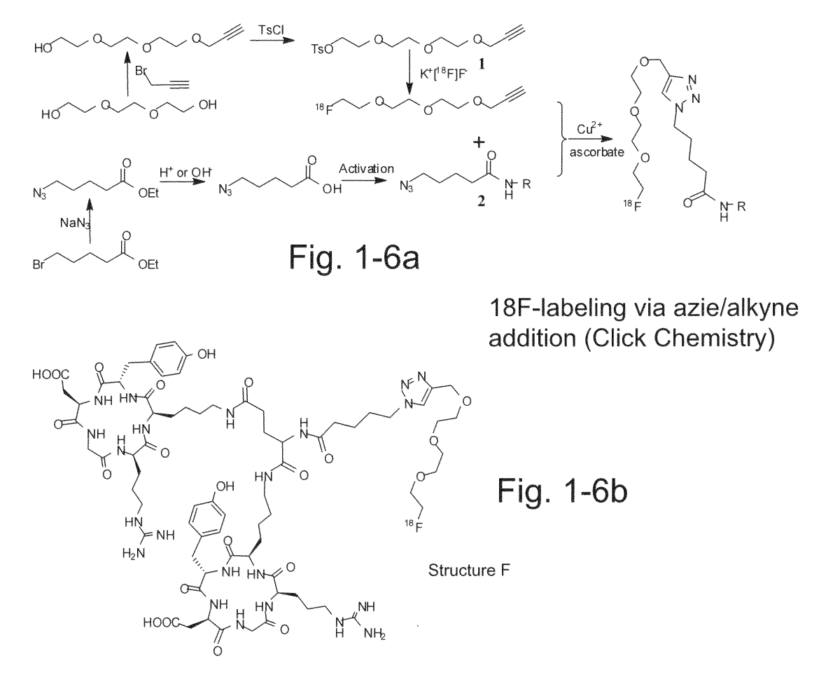

The RGD compounds can include a multimeric RGD peptide, a tag, and a linker connecting the multimeric RGD peptide and the tag. FIG. 1-1a illustrates an embodiment of an RGD compound. “Circle X” is the tag and “rectangle R” is one or more linkers. FIGS. 1-1b to 1-1d illustrate embodiments of RGD compounds having an RGD dimer (FIG. 1-1b), an RGD tetramer (FIG. 1-1c), and an RGD octamer (FIG. 1-1d). Additional details regarding the RGD compound is described below and in the Examples.

The RGD compounds can be imaged using one or more types of imaging systems. The imaging systems can include, but are not limited to, optical systems, magnetic systems, x-ray systems, nuclear systems, positron emission tomography (PET) imaging systems, ultrasound systems, and the like. In particular, the imaging techniques can include, but are not limited to, NIR fluorescence, intravital microscopy, X-ray computed tomography (CT), magnetic resonance imaging (MRI), ultrasound (ULT), single photon emission computed tomography (SPECT), PET, and combinations thereof. In an embodiment, PET imaging is a preferred embodiment.

### Multimeric RGD Peptide

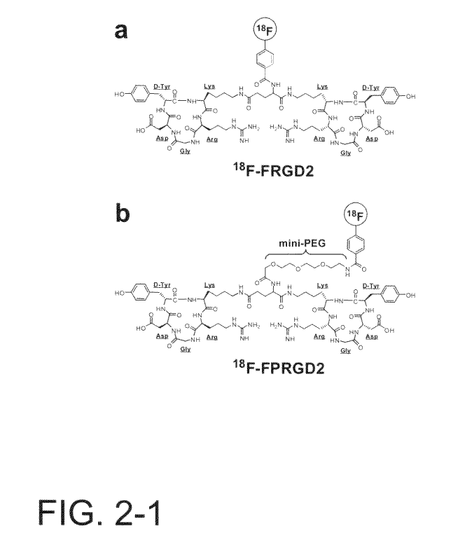

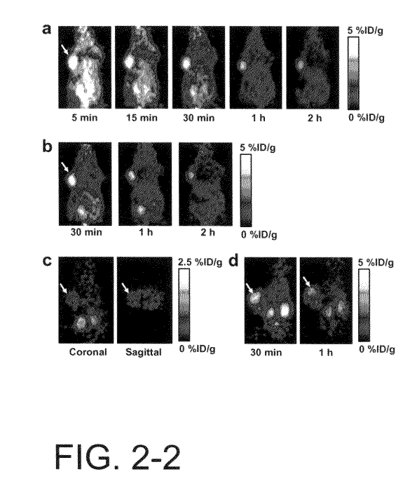

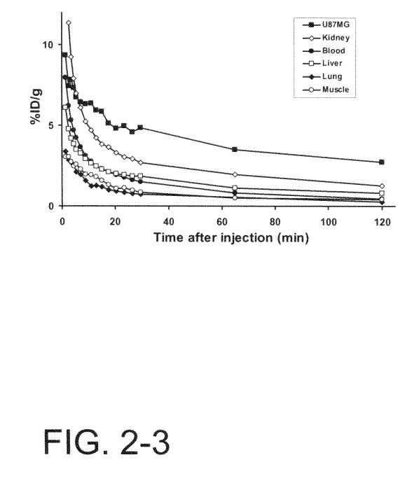

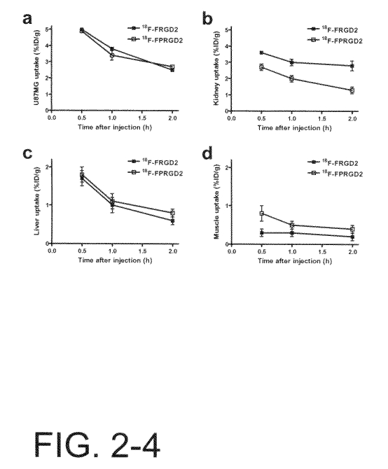

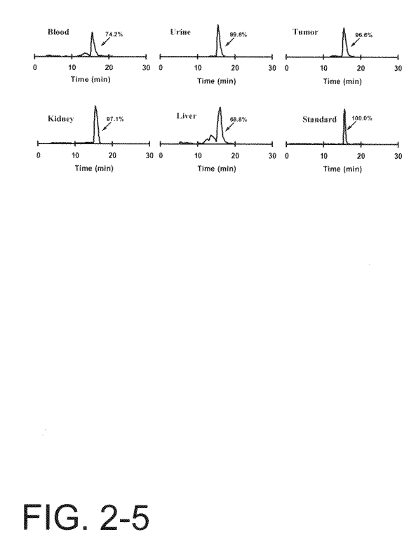

The multimeric RGD peptide can included 2 or more (e.g., 3, 4, 5, 6, 7, 8, or more) RGD peptide units (See, FIG. 1-2a). The the RGD peptide unit can be a cyclic peptide containing the Arg-Gly-Asp amino acid sequence. The term “cyclic peptide” refers to a head-to-tail cyclized peptide and/or a cyclized peptide via one or more disulfide bonds. In an embodiment, the multimeric RGD peptide includes, but is not limited to, RGD dimer peptides (E[c(RGDyK)]₂, FIG. 1-2b), RGD tetramer peptides (FIG. 1-2c, E{E[c(RGDyK)]₂}₂), and RGD octamer peptides (FIG. 1-2d, E{E{E[c(RGDyK)]₂}₂}₂).

### Tag

In an embodiment the tag can include, but is not limited to, a detecting unit and/or a therapeutic unit. In an embodiment, the RGD compound can include both a detecting unit and/or a therapeutic unit with one or more linkers between or among the multimeric RGD peptide, the detecting unit, and/or the therapeutic unit.

In an embodiment, the RGD compound includes one or more detecting units that can be used to detect, image, or otherwise identify the RGD compound, quantify the amount of RGD compound, determine the location of the RGD compound (e.g., in imaging), and combinations thereof. The detecting unit can be an element or a compound that can be detected using PET, SPECT, NIR fluorescence, ultrasound, and magnetic resonance.

In an embodiment, the detecting unit can include a radiolabel and/or a compound or chelating agent including a radiolabel. In an embodiment, the radiolabel (e.g., non-radiolabels and their radiolabel counterparts) can include, but is not limited to, F-19 (F-18), C-12 (C-11), I-127 (I-125, I-124, I-131, I-123), CI-36 (CI-32, CI-33, CI-34), Br-80 (Br-74, Br-75, Br-76, Br-77, Br-78), Re-185/187 (Re-186, Re-188), Y-89 (Y-90, Y-86), Lu-177, or Sm-153. It should be noted that an alternative way to represent F-18, C-11, and the like, is the following: ¹⁸F and ¹¹C respectively, and both ways are used herein. In an embodiment, the radiolabel can be ¹¹C, ¹⁸F, ⁷⁶Br, ¹²³I, ¹²⁴I, or ¹³¹I. In an embodiment, the radiolabel can be ¹⁸F, ⁷⁶Br, or ¹²³I, ¹²⁴I or ¹³¹I, which are suitable for use in peripheral medical facilities and PET clinics. In particular embodiments, the radiolabel or PET isotope can include, but is not limited to, ⁶⁴Cu, ¹²⁴I, ^{76/77}Br, ⁸⁶Y, ⁸⁹Zr, or ⁶⁸Ga. Embodiments for attaching the isotopes are described in the Specification and in the Examples.

In an exemplary embodiment, the PET isotope is ¹⁸F. Fluorine-18 (t_{1/2}=109.7 min; β⁺, 99%) is an ideal short-lived PET isotope for labeling small molecular recognition units such as antigen binding domain of antibody fragments and small biologically active peptides. ¹⁸F-labeled prosthetic groups such as N-succinimidyl 4-¹⁸F-fluorobenzoate (¹⁸F-SFB) have been developed that can be attached to either N-terminal or lysine ε-amino groups with little or no loss of bioactivity of the peptide ligand.

In an embodiment of the present disclosure, X can be a SPECT isotope. The SPECT isotope can include, but is not limited to, ¹²³I, ¹²⁵I, ¹³¹I, ⁹⁹Tc, ¹¹¹In, ^{186/188}Re, or combinations thereof.

In an embodiment, the RGD compound includes one or more therapeutic units that can be used to treat a disease, a condition, an injury, or a related biological event, activity, and/or function. The therapeutic unit includes, but is not limited to, alpha-emitting radionuclides (e.g., At-211, Bi-212, Bi-213, Ra-223, and Ac-225) and beta-emitting radionuclides (e.g., Cu-67, Y-90, Ag-111, I-131, Pm-149, Sm-153, Ho-166, Lu-177, Re-186, and Re-188). In embodiment, the therapeutic unit is a chemotherapeutic unit. The chemotherapeutic unit can include, but is not limited to, paclitaxel, doxorubicin, methotrexate, chlorambucil, and/or 5-fluorodeoxyuridine.

In some embodiments a chelator compound can be used to connect the tag to the multimeric RGD peptide or can be used to chelate the radiolabel and then the chelator can be connected (e.g., a linker) to the multimeric RGD peptide. The chelator compound can include, but is not limited to, a macrocyclic chelator, a non-cyclic chelator, and combinations thereof, as well as those shown in the figures. The macrocyclic chelator can include, but is not limited to, 1,4,7,10-tetraazadodecane-N,N′,N″,N′″-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), diethylenetriaminepentaacetic (DTPA), 4,11-bis(carboxymethyl)-1,4,8,11-tetraazabicyclo[6.6.2]hexadecane (CB-TE2A), 1-N-(4-aminobenzyl)-3,6,10,13,16,19-hexaazabicyclo[6.6.6]eicosane-1,8-diamine (SarAr), or combinations thereof.

Additional chelators include natural chelators and synthetic chelators. The natural chelators include, but are not limited to, carbohydrates (e.g., polysaccharides), organic acids with more than one coordination group, lipids, steroids, amino acids, peptides, phosphates, nucleotides, tetrapyrrols, ferrioxamines, lonophores (e.g., gramicidin, monensin, and valinomycin), and phenolics. The synthetic chelator include, but are not limited to, ammonium citrate dibasic, ammonium oxalate monohydrate, ammonium tartrate dibasic, ammonium tartrate dibasic solution, pyromellitic acid, calcium citrate tribasic tetrahydrate, ethylene glycol-bis(2-aminoethylether)-N,N,N,N-tetra acetic acid, sodium glycocholate, ammonium citrate dibasic, calcium citrate tribasic tetrahydrate, magnesium citrate tribasic, potassium citrate, sodium citrate monobasic, lithium citrate tribasic, sodium citrate tribasic, citric acid, N,N-dimethyldecylamine-N-oxide, N,N-dimethyldodecylamine-N-oxide, ammonium citrate dibasic, ammonium tartrate dibasic, ethylenediaminetetraacetic acid diammonium salt, potassium D-tartrate monobasic, N,N-dimethyldecylamine-N-oxide, N,N-dimethyldodecylamine-N-oxide, ethylenediaminetetraacetic acid dipotassium salt dihydrate, sodium tartrate dibasic, ethylenediaminetetraacetic acid, ethylenediaminetetraacetic acid disodium salt dihydrate, ethylenediaminetetraacetic acid, ethylenediaminetetraacetic acid tetrasodium salt hydrate, ethylenediaminetetraacetic acid tripotassium salt, ethylene glycol-bis(2-aminoethylether)-N,N,N,N-tetra acetic acid, ethylene glycol-bis(2-aminoethylether)-N,N,N,N-tetra acetic acid, potassium oxalate, sodium oxalate, ethylene glycol-bis(2-aminoethylether)-N,N,N,N-tetra acetic acid, ethylenediaminetetraacetic acid, ethylenediaminetetraacetic acid diammonium salt, ethylenediaminetetraacetic acid dipotassium salt dihydrate, ethylenediaminetetraacetic acid disodium salt dihydrate, ethylenediaminetetraacetic acid disodium salt dihydrate, ethylenediaminetetraacetic acid, ethylenediaminetetraacetic acid tetrasodium salt hydrate, ethylenediaminetetraacetic acid tripotassium salt, ethylenediaminetetraacetic acid trisodium salt trihydrate, ethylenediaminetetraacetic acid dipotassium salt dihydrate, ethylene glycol-bis(2-aminoethylether)-N,N,N,N-tetra acetic, ethylene glycol-bis(2-aminoethylether)-N,N,N,N-tetra acetic acid, sodium glycocholate, ethylene glycol-bis(2-aminoethylether)-N,N,N,N-tetra acetic acid, 5-sulfosalicylic acid, N,N-dimethyldodecylamine-N-oxide, magnesium citrate tribasic, magnesium citrate tribasic nonahydrate, ammonium oxalate monohydrate, potassium tetraoxalate, potassium oxalate, sodium oxalate, potassium citrate, ethylenediaminetetraacetic acid dipotassium salt dihydrate, potassium D-tartrate monobasic, potassium peroxodisulfate, potassium citrate monobasic, potassium citrate tribasic, potassium oxalate monohydrate, potassium peroxodisulfate, potassium sodium tartrate, potassium sodium tartrate tetrahydrate, potassium D-tartrate monobasic, potassium tetraoxalate dihydrate, pyromellitic acid hydrate, potassium sodium tartrate, potassium sodium tartrate, ethylenediaminetetraacetic acid disodium salt dihydrate, sodium citrate monobasic, sodium bitartrate, sodium tartrate dibasic, sodium bitartrate monohydrate, sodium citrate monobasic, sodium citrate tribasic dihydrate, sodium citrate tribasic, sodium glycocholate hydrate, sodium oxalate, sodium tartrate dibasic dihydrate, sodium tartrate dibasic, 5-sulfosalicylic acid dihydrate, ammonium tartrate dibasic, sodium tartrate dibasic, potassium D-tartrate monobasic, sodium bitartrate, potassium sodium tartrate, L-(+)-tartaric acid, ethylenediaminetetraacetic acid tetrasodium salt hydrate, L-(+)-tartaric acid, calcium citrate tribasic tetrahydrate, sodium glycocholate, lithium citrate tribasic, magnesium citrate tribasic, ethylenediaminetetraacetic acid tripotassium salt, sodium citrate tribasic, and ethylenediaminetetraacetic acid trisodium salt trihydrate. In particular, the chelator compound can include, but is not limited to, EDTA (ethylenediaminetetraacetic acid), DTPA (diethylenetriaminepentaacetate), DOPA (dihydroxyphenylalanine), and derivatives of each. The agent can be incorporated into the chelate compound using methods such as, but not limited to, direct incorporation, template synthesis, and/or transmetallation, as well as methods described in the Examples.

In an embodiment, the chelator can include, but is not limited to, DOTA, NOTA, EDTA, TETA, SarAr, CB-TE2A, 6-hydrazinonicotinic (HYNIC), NxSy chelates (e.g., diamide dithiolate ligand system (N2S2) and dimethylglycyl-L-seryl-L-cysteinylglycinamide (N3S)), or mercapto acetyl tri-glycine (MAG3) ligands. The NxSy chelates include bifunctional chelators that are capable of coordinately binding a metal or radiometal (See, Proc. Natl. Acad. Sci. USA 85:4024-29, 1988; Bioconj. Chem. 1:431-37, 1990; and in the references cited therein, each of which incorporated herein by reference for the corresponding discussion). In an embodiment, the radiolabel used in conjunction with the chelator can include, but is not limited to, ^{60/61/62/64/67}Cu, ^{67/68}Ga, ^{86/88/90}Y, ¹⁷⁷Lu, ^{212/213}Bi, ¹⁵³Gd, ^{149/161}Tb, ^{157/165}Dy, ^{165/169/171}Er, ¹⁶⁷Tm, ¹⁶⁹Yb, ¹⁵³Sm, ¹⁶⁶Ho, ¹¹¹In, ^{94m/99m}Tc.

FIG. 1-3a illustrates embodiments of the tag. As noted above in reference to FIGS. 1-1a to 1-1d, “circle X” is the tag. “X” without the circle is a radiolabel such as those described above or those noted in FIG. 1-3a. R1 can be any one or a combination of the groups (e.g., alkane, poly(ethylene glycol)(PEG), or aromatic ring, wherein n=1-20 (e.g., 1 in an embodiment) and m=1-10) noted in FIG. 1-3a. Y can be any one or a combination of the groups (attached to the R1 in the listing and can include an active ester, aldehyde, thiol, maleimide, alkyne, azide, hydrazone, or amine) noted in FIG. 1-3a. R2 can be any one or a combination of the groups noted in FIG. 1-3a.

FIG. 1-3b illustrates embodiments of the tag. As noted above in reference to FIGS. 1-1a to 1-1d, “circle X” is the tag. “X” without the circle is a radiolabel such as those described above or those noted in FIG. 1-3b. R1 can be any one or a combination of the groups (e.g., alkane, poly(ethylene glycol)), and aromatic ring, wherein n=1-20 (e.g., 1 in an embodiment) and m=1-10) noted in FIG. 1-3b. Y can be any one or a combination of the groups (attached to the R1 in the listing and can include an active ester, aldehyde, thiol, maleimide, alkyne, azide, hydrazone, or amine)) noted in FIG. 1-3b. R2 can be any one or a combination of the groups noted in FIG. 1-3b.

### Linker

In an embodiment, the linker is one or more compounds and/or peptides that connects one or more portions of the RGD compound by bonding (e.g., chemically, biochemically, physically, combinations, or otherwise) to two or more of the components of the RGD compound. In an embodiment, the linker connects the multimeric RGD peptide to the tag. In an embodiment, the linker is one compound or peptide or two or more compound or peptides. In an embodiment, the linker can be a carbohydrate, a peptide, and/or a PEG (e.g., mini PEG, having a molecular weight of about 200 to 20,000).

FIG. 1-4a illustrates an embodiment of a linker. FIG. 1-4a illustrates a carbohydrate linker. R1 can be any one or a combination of the groups (e.g., alkane, poly(ethylene glycol), or aromatic ring, wherein n=1-20 (e.g., 1 in an embodiment) and m=1-10) noted in FIG. 1-4a. Y and Z can be any one or a combination of the groups (attached to the R1 in the listing and can include an active ester, aldehyde, thiol, maleimide, alkyne, azide, hydrazone, or amine) noted in FIG. 1-4a. R2 can be any one or a combination of the groups noted in FIG. 1-4a. It should be noted that Y and Z should not be the same in the same carbohydrate bifunctional linker.

FIG. 1-4b illustrates an embodiment of a linker. FIG. 1-4b illustrates a poly(ethylene glycol) linker, where N=0 to 50 (in an embodiment, N is 1, 2, or 3). In an embodiment, the PEG is a mini-PEG having a molecular weight of about 200 to 20,000 or about 200 to 2000. X and Y include, but are not limited to, an active ester, aldehyde, thiol, maleimide, alkyne, azide, hydrazone, or amine. It should be noted that Y and Z may be the same or different in the same PEG bifunctional linker.

### RGD Compounds

RGD compounds include compounds such as, but not limited to, an RGD compound having a schematic structure shown in FIG. 1-5a, which is ¹⁸F-labeled RGD dimer via 4-fluorobenzoyl prosthetic group (¹⁸F-FRGD2); an RGD compound having a schematic structure shown in FIG. 1-5b, which is a miniPEG-RGD dimer via 4-fluorobenzoyl prosthetic group (¹⁸F-FPRGD2); an RGD compound having a schematic structure shown in FIG. 1-5c which is ¹⁸F-labeled miniPEG-RGD tetramer via 4-fluorobenzoyl prosthetic group (¹⁸F-FPRGD4); an RGD compound having a schematic structure shown in FIG. 1-5d, which is a DOTA conjugated RGD tetramer (DOTA-RGD tetramer); an RGD compound having a schematic structure shown in FIG. 1-5d, which is an octamer for ⁶⁴Cu-labeling (DOTA-RGD octamer); and an RGD compound having a schematic structure shown in FIG. 1-6b, which is a dimeric RGD peptide labeled with F-18 via click chemistry. FIG. 1-6a illustrates a method (click chemistry) for preparing the RGD compound shown in FIG. 1-6b.

### Method of Making RGD Compounds

The RGD compounds can be made using one or more methods or processes (e.g., click chemistry, Michael addition processes, and the like). Details regarding some exemplar methods are shown in the Examples.

In an embodiment, the RGD compound can be made using click chemistry, in which the RGD peptide is derivatized with azide functional group and then reacted with a ¹⁸F-labeled alkyne following a Cu(I)-catalyzed Huisgen cycloaddition to form 1,2,3-triazoles. Additional details are described in the Examples.

In an embodiment, the RGD compound can be made via Michael addition processes, in which a thiolated RGD peptide is reacted with a thiol-reactive synthon, N-[2-(4-¹⁸F-fluorobenzamido)ethyl]maleimide (¹⁸F-FBEM) to form a stable thiol ether. Additional details are described in the Examples.

### Methods of Use

Embodiments of this disclosure include, but are not limited to: methods of imaging tissue, cells, or a host using an RGD compound; methods of imaging an angiogenesis related disease or related biological events; methods of treating an angiogenesis related disease or related biological events; methods of diagnosing an angiogenesis related disease or related biological events; methods of monitoring the progress of an angiogenesis related disease or related biological events, and the like.

Embodiments of the present disclosure can be used to image, detect, study, monitor, evaluate, and/or screen, the angiogenesis related diseases or related biological events in vivo or in vitro using an RGD compound.

In general, the RGD compound can be used in imaging angiogenesis related diseases. For example, the labeled RGD peptide is provided or administered to a host in an amount effective to result in uptake of the compound into the angiogenesis related disease or tissue of interest. The host is then introduced to an appropriate imaging system (e.g., PET system) for a certain amount of time. The angiogenesis related disease that takes up the RGD compound could be detected using the imaging system.

In an embodiment, the RGD compound may find use both in diagnosing and/or in treating precancerous tissue, cancer, and/or tumors. In diagnosing the presence of precancerous tissue, cancer, and/or tumors in a host, the RGD compound is administered to the host in an amount effective to result in uptake of the RGD compound into the precancerous tissue, cancer, and/or tumors. After administration of the RGD compound, the precancerous tissue, cancer, and/or tumors that takes up the RGD compound is detected using an appropriate imaging system. Embodiments of the present disclosure can non-invasively image the precancerous tissue, cancer, and/or tumors throughout an animal or patient.

In another embodiment, the RGD compound can be used in treating angiogenesis related disease that has been previously diagnosed by a method described herein or by another method. The RGD compound finds use in both surgical treatment and in chemical treatment of angiogenesis related disease. In a host where angiogenesis related disease tissue or cells are to be surgically removed, the RGD compound is administered prior to and/or coincident with the surgical procedure. The host is exposed to the appropriate imaging system and an attending medical provider can then directly visualize the angiogenesis related disease.

The RGD compound can also find use in a host undergoing chemotherapy, to aid in visualizing the response of angiogenesis related disease to the treatment. In this embodiment, the RGD compound is typically visualized and sized prior to treatment, and periodically during chemotherapy to monitor the tumor size and the change of integrin expression level during the treatment.

The RGD compound also finds use as a screening tool in vitro to select compounds for use in treating angiogenesis related diseased tissue or cells. The angiogenesis related disease could be easily monitored by incubating the cells with the RGD compound during or after incubation with one or more candidate drugs. The ability of the drug compound to affect the binding of suitably labeled RGD compound (e.g., RGD peptide) will confer potency of the drug.

It should be noted that the amount effective to result in uptake of a RGD compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Typical hosts to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood, urine, or tissue samples of the animals mentioned for veterinary applications.

### Kits

The present disclosure also provides packaged compositions or pharmaceutical compositions comprising a pharmaceutically acceptable carrier and an RGD compound of the disclosure. In certain embodiments, the packaged compositions or pharmaceutical composition includes the reaction precursors to be used to generate the imaging compound according to the present disclosure. Other packaged compositions or pharmaceutical compositions provided by the present disclosure further include indicia including at least one of: instructions for using the composition to image a host, or host samples (e.g., cells or tissues), which can be used as an indicator of conditions including, but not limited to, angiogenesis related disease and biological related events. In embodiments, the kit may include instructions for using the composition or pharmaceutical composition to assess therapeutic effect of a drug protocol administered to a patient, instructions for using the composition to selectively image malignant cells and tumors, and instructions for using the composition to predict metastatic potential.

This disclosure encompasses kits that include, but are not limited to, the RGD compound and directions (written instructions for their use). The components listed above can be tailored to the particular biological event to be monitored as described herein. The kit can further include appropriate buffers and reagents known in the art for administering various combinations of the components listed above to the host cell or host organism. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Dosage Forms

Embodiments of the present disclosure can be included in one or more of the dosage forms mentioned herein. Unit dosage forms of the pharmaceutical compositions (the “composition” includes at least the RGD compound) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

Like the amounts and types of excipients, the amounts and specific type of active ingredient in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Introduction

We have previously reported that 18F-FB-E[c(RGDyK)]2 (18F-FRGD2) allows quantitative PET imaging of integrin αvβ3 expression. However, the potential clinical translation was hampered by the relatively low radiochemical yield. The goal of this study was to improve the radiolabeling yield, without compromising the tumor targeting efficiency and in vivo kinetics, by incorporating a hydrophilic bifunctional mini-PEG spacer.

In this Example, we incorporated a mini-PEG spacer, 11-amino-3,6,9-trioxaundecanoic acid, with three ethylene oxide units, onto the glutamate α-amino group of the dimeric RGD peptide E[c(RGDyK)]₂ (denoted as RGD2). The hypothesis was that the mini-PEG will increase the overall hydrophilicity and alleviate the steric hindrance thereby increase the ¹⁸F-labeling yield. The mini-PEG spacered dimeric RGD peptide was labeled with ¹⁸F through ¹⁸F-SFB and evaluated in murine tumor models by microPET imaging. Extensive in vitro, ex vivo, and in vivo experiments were carried out to evaluate the tumor targeting efficacy and pharmacokinetics of ¹⁸FPRGD2, which was compared with previously reported ¹⁸F-FRGD2. ¹⁸F-FB-mini-PEG-E[c(RGDyK)]₂ (¹⁸F-FPRGD2) was synthesized by coupling N-succinimidyl-4-¹⁸F-fluorobenzoate (¹⁸F-SFB) with NH₂-mini-PEG-E[c(RGDyK)]₂ (denoted as PRGD2). In vitro receptor binding assay, metabolic stability, integrin αᵥβ₃ specificity of the new tracer ¹⁸F-FPRGD2 was assessed. The diagnostic value of ¹⁸F-FPRGD2 was evaluated in subcutaneous U87MG glioblastoma xenografted mice and in c-neu transgenic mice by quantitative microPET imaging studies. The decay-corrected radiochemical yield based on ¹⁸F-SFB was over 60% with radiochemical purity of >99%. ¹⁸F-FPRGD2 had high receptor-binding affinity, metabolic stability and integrin αᵥβ₃-specific tumor uptake in U87MG glioma xenograft model comparable to those of ¹⁸F-FRGD2. The kidney uptake was appreciably lower for ¹⁸F-FPRGD2 compared with ¹⁸F-FRGD2 (2.0±0.2% ID/g for ¹⁸F-FPRGD2 vs. 3.0±0.2% ID/g ¹⁸F-FRGD2 at 1 h postinjection (p.i.)). The uptake in all the other organs except in the urinary bladder was at background level. ¹⁸F-FPRGD2 also exhibited excellent tumor uptake in c-neu oncomice (3.6±0.1% ID/g at 30 min p.i.).

Incorporation of a mini-PEG spacer significantly improved the overall radiolabeling yield of ¹⁸F-FPRGD2. ¹⁸F-FPRGD2 also had reduced renal uptake and similar tumor targeting efficacy as compared to ¹⁸F-FRGD2. Further test and clinical translation of ¹⁸F-FPRGD2 is warranted.

### Materials and Methods

All chemicals obtained commercially were of analytical grade and used without further purification. No-carrier-added ¹⁸F-F⁻ was obtained from in-house PETtrace cyclotron (GE Healthcare). The semi-preparative reversed-phase HPLC system was the same as reported previously (J Nucl Med 2006;47:113-121, which is incorporated herein by reference for the corresponding discussion). With a flow rate of 5 mL/min, the mobile phase was changed from 95% solvent A (0.1% trifluoroacetic acid [TFA] in water) and 5% solvent B (0.1% TFA in acetonitrile, ACN) (0-2 min) to 35% solvent A and 65% solvent B at 32 min. Analytical HPLC has the same gradient system except that the flow rate was 1 mL/min. The UV absorbance was monitored at 218 nm and the identification of the peptides was confirmed based on the UV spectrum acquired using a PDA detector. C₁₈ Sep-Pak cartridges (Waters) were pretreated with ethanol and water before use.

Synthesis of NH₂-mini-PEG-E[c(RGDyK)]₂

To a solution of 40 mg (0.13 mmol) Boc-11-amino-3,6,9-trioxaundecanoic acid (Boc-NH-mini-PEG-COOH) and 20 μL N,N′-Diisopropylethylamine (DIPEA) in ACN was added O-(N-Succinimidyl)-1,1,3,3-tetramethyl-uronium tetrafluoroborate (TSTU, 27 mg, 0.09 mmol). The reaction mixture was stirred at room temperature for 0.5 h and then added to 25 mg (0.02 mmol) of E[c(RGDyK)]₂ in N,N′-dimethylformamide (DMF). After being stirred at room temperature for 2 h, the desired product Boc-NH-mini-PEG-E[c(RGDyK)]₂ was isolated by semi-preparative HPLC. The Boc-group was then removed with anhydrous TFA and the crude product was again purified by semi-preparative HPLC. The collected fractions were combined and lyophilized to afford NH₂-mini-PEG-E[c(RGDyK)]₂ (abbreviated as PRGD2) as a white fluffy powder.

Synthesis of FB-NH-mini-PEG-E[c(RGDyK)]₂

SFB (4 mg, 16.8 μmol) and PRGD2 (2 mg, 1.3 μmol) were mixed in 0.05 mol/L borate buffer (pH 8.5) at room temperature. After constant shaking for 2 h, the desired product FB-NH-mini-PEG-E[c(RGDyK)]₂ (abbreviated as FPRGD2) was isolated by semi-preparative HPLC.

### Cell Binding Assay

In vitro integrin αᵥβ₃-binding affinity and specificity of PRGD2 and FPRGD2 were assessed via competitive cell binding assay using ¹²⁵I-echistatin as the integrin αᵥβ₃-specific radioligand (J Nucl Med 2005;46:1707-1718 and J Nucl Med 2006;47:1172-1180, each of which is incorporated herein by reference for the corresponding discussion). Experiments were performed on U87MG human glioblastoma cells with triplicate samples as previously reported. The best-fit 50% inhibitory concentration (IC₅₀) values for the U87MG cells were calculated by fitting the data with nonlinear regression using Graph-Pad Prism (GraphPad Software, Inc.) and compared to those of RGD2 and FRGD2.

### Radiochemistry

¹⁸F-SFB was synthesized as previously reported with HPLC purification [21, 23] (Eur J Nucl Med Mol Imaging 2004;31:1081-1089 and J Nucl Med 2007, each of which is incorporated herein by reference for the corresponding discussion). Recently, we incorporated ¹⁸F-SFB synthesis into a commercially available synthetic module (TRACERIab FX_{FN}; GE) with automatic computer control. The purified ¹⁸F-SFB was rotary evaporated to dryness, re-dissolved in dimethyl sulfoxide (DMSO, 200 μL), and added to a DMSO solution of PRGD2 (200 μg, 0.12 μmol) and DIPEA (20 μL). The reaction mixture was allowed to incubate at 60° C. for 30 min. After dilution with 4 mL of water with 0.1% TFA, the mixture was injected onto the semi-preparative HPLC. The collected fractions containing ¹⁸F-FPRGD2 (FIG. 2-1b) were combined and rotary evaporated to remove ACN and TFA. The activity was then reconstituted in normal saline and passed through a 0.22-μm Millipore filter into a sterile multidose vial for in vivo experiments.

### Octanol-Water Partition Coefficient

Approximately 111 kBq of ¹⁸F-FPRGD2 in 500 μL of PBS (pH 7.4) were added to 500 μL of octanol in an Eppendorf microcentrifuge tube. The mixture was vigorously vortexed for 1 min at room temperature. After centrifugation at 12,500 rpm for 5 min in an Eppendorf microcentrifuge, 100 μL aliquots of both layers were pipetted and the radioactivity was measured using a γ-counter (Packard). The experiment was carried out in triplicates.

### Cell Line and Animal Models

U87MG cells were grown in Dulbecco's medium (Gibco) supplemented with 10% fetal bovine serum (FBS), 100 IU/mL penicillin, and 100 μg/mL streptomycin (Invitrogen Co.), at 37° C. in a humidified atmosphere containing 5% CO₂. All animal experiments were performed under a protocol approved by Stanford's Administrative Panel on Laboratory Animal Care. The subcutaneous U87MG tumor model was generated by injection of 5×10⁶ cells in 50 mL PBS into the shoulder of female athymic nude mice (Harlan, Indianapolis, Ind.). The mice were subjected to microPET studies when the tumor volume reached 100-300 mm³ (3-4 weeks after inoculation) (J Nucl Med 2006;47:2048-2056 and Cancer Res 2006;66:9673-9681, each of which is incorporated herein by reference for the corresponding discussion). The c-neu oncomouse (Charles River Laboratories, Charles River, Canada) is a spontaneous tumor-bearing model that carries an activated c-neu oncogene driven by a mouse mammary tumor virus (MMTV) promoter (Cell 1988;54:105-115, which is incorporated herein by reference for the corresponding discussion). Transgenic mice uniformly expressing the MMTV/c-neu gene develop mammary adenocarcinomas between 4 and 8 months postpartum that involve the entire epithelium in each gland. These mice were subjected to microPET scans at about 8 months old and the tumor volume were about 300-500 mm³.

### MicroPET Imaging

PET scans and image analysis were performed using a microPET R4 rodent model scanner (Siemens Medical Solutions) as previously reported (J Nucl Med 2006;47:113-121 and J Nucl Med 2005;46:1707-1718, each of which is incorporated herein by reference for the corresponding discussion). Each mouse was tail-vein injected with about 3.7 MBq (100 μCi) of ¹⁸F-FPRGD2 under isoflurane anesthesia. The 30-min dynamic scan (1×30 s, 4×1 min, 1×1.5 min, 4×2 min, 1×2.5 min, 4×3 min, total of 15 frames) was started 1 min after injection. Five min static PET images were also acquired at 1 h and 2 h post-injection (p.i.). The images were reconstructed by a 2-dimensional ordered-subsets expectation maximum (OSEM) algorithm and no correction was applied for attenuation or scatter. For blocking experiment, the tumor mice were co-injected with 10 mg/kg mouse body weight of c(RGDyK) and 3.7 MBq of ¹⁸F-FPRGD2 and 5 min static PET scans were then acquired at 1 h p.i.

### Metabolic Stability of ¹⁸F-FPRGD2

A U87MG tumor mouse was intravenously injected with 3.7 MBq of ¹⁸F-FPRGD2. At 1 h after injection, the mouse was sacrificed, the blood, urine, liver, kidneys, and the U87MG tumor were collected and metabolite analysis was carried out as previously reported (J Nucl Med 2006;47: 113-121 and J Nucl Med 2006;47:1172-1180, each of which is incorporated herein by reference for the corresponding discussion). In brief, blood sample was immediately centrifuged for 5 min at 13,200 rpm. Other tissues were homogenized and then centrifuged for 5 min at 13,200 rpm. The supernatant was each passed through a C₁₈ Sep-Pak cartridge. The urine sample was directly diluted with 1 mL of PBS and passed through a C₁₈ Sep-Pak cartridge. The cartridges were each washed with 2 mL of water and eluted with 2 mL of ACN containing 0.1% TFA. The ACN eluent was concentrated and injected onto the analytical HPLC. The eluent was collected with a fraction collector (0.5 min/fraction) and the radioactivity of each fraction was measured with the γ-counter.

### Statistical Analysis

Quantitative data were expressed as mean±SD. Means were compared using One-way ANOVA and student's t-test. P values<0.05 were considered statistically significant.

### Results

### Chemistry

PRGD2 was synthesized with an overall yield of 64% (HPLC Rₜ: 12.2 min; MALDI-TOF-MS: C₆₇H₁₀₃N₂₀O₂₂, calculated 1539.7, observed 1540.1). FPRGD2 was prepared with 69% yield (HPLC Rₜ: 15.8 min; MALDI-TOF-MS: C₇₄H₁₀₆FN₂₀O₂₃, calculated 1662.7, observed 1662.8).

The total time for ¹⁸F-SFB synthesis was about 100 min and the decay-corrected yield was 67%±11% (n=10). The yield of ¹⁸F-SFB coupling with PRGD2 is dependent on the peptide concentration, temperature, pH, solvent and reaction time. After systematic investigation and optimization, 200 μg of PRGD2 was used for each reaction. The highest yield was achieved in DMSO with 20 μL DIPEA as the base. The decay-corrected radiochemical yield based on ¹⁸F-SFB was over 60% (n=3), significantly higher than the yield for ¹⁸F-FRGD2 (maximum 23%, average 4-6%). The radiochemical purity of ¹⁸F-FPRGD2 was >99% according to analytical HPLC and the specific activity was about 100-200 TBq/mmol. Starting from ¹⁸F-F⁻, the total synthesis time of ¹⁸F-FPRGD2 was about 180 min and the overall decay-corrected yield was over 40%. The much improved synthesis yield of ¹⁸F-FPRGD2 makes it feasible for clinical translation. For example, starting from 37 GBq (1 Ci) of ¹⁸F-F⁻, about 4-5 GBq (100-140 mCi) of ¹⁸F-FPRGD2 can be synthesized in 3 h (enough for 3-5 patients).

The octanol/water partition coefficient (logP) for ¹⁸F-FPRGD2 was −2.28±0.05 (¹⁸F-FRGD2: −2.10±0.03), indicating that the tracer is slightly more hydrophilic than ¹⁸F-FRGD2 after incorporation of the mini-PEG spacer.

### Cell Binding Assay

The receptor-binding affinity of PRGD2 and FPRGD2 was evaluated using U87MG cells (integrin αᵥβ₃-positive). Both peptides inhibited the binding of ¹²⁵I-echistatin (integrin αᵥβ₃ specific) to U87MG cells in a concentration dependent manner. The IC₅₀ values for PRGD2 and FPRGD2 were 70.1±3.5 and 40.6±4.6 nmol/L (n=3) respectively, comparable to that of FRGD2 (55.1±6.5 nmol/L). Due to the presence of the mini-PEG linker and/or the prosthetic group (FB), all three peptides had slightly lower binding affinity than RGD2 (IC₅₀=26.1±3.2 nmol/L). The comparable IC₅₀ values of FRGD2 and FPRGD2 suggest that incorporation of a mini-PEG linker had minimal effect on the receptor binding. It is of note that cell-based receptor binding assay typically give higher IC₅₀ values (lower binding affinity) than those measured by ELISA or solid-phase receptor binding assay. Therefore, when comparing the receptor binding affinity (IC₅₀ values), it is critical that the IC₅₀ values were obtained from the same assay.

### MicroPET Imaging Study

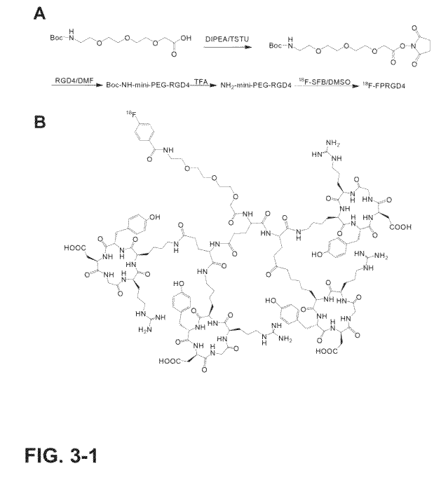

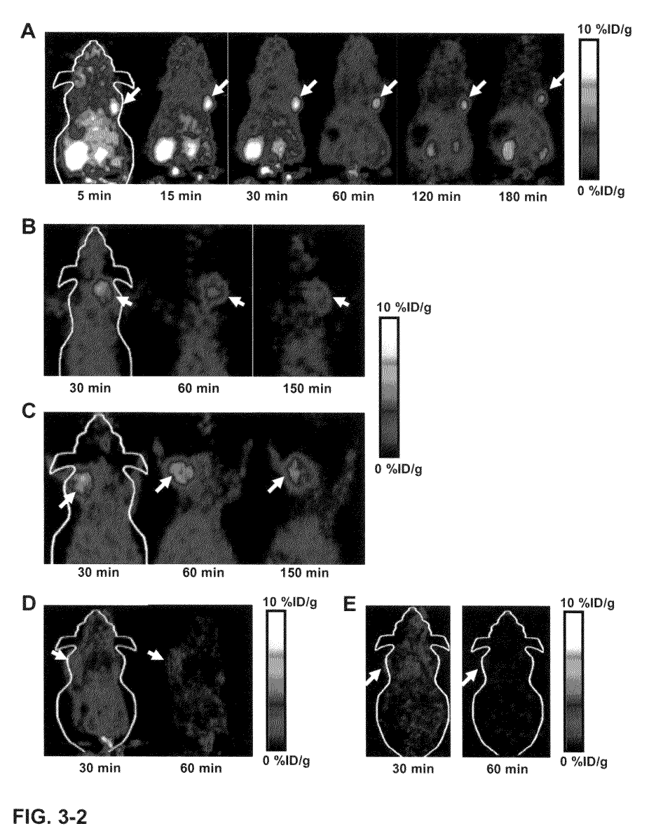

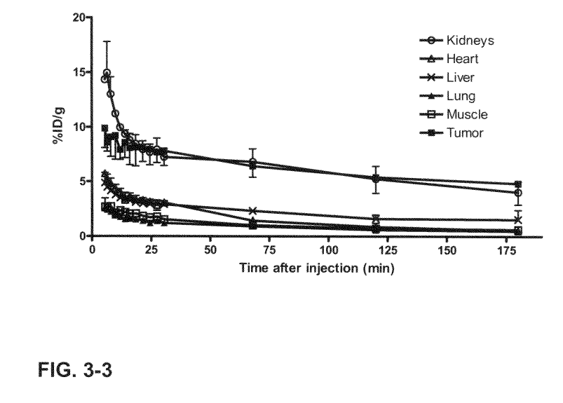

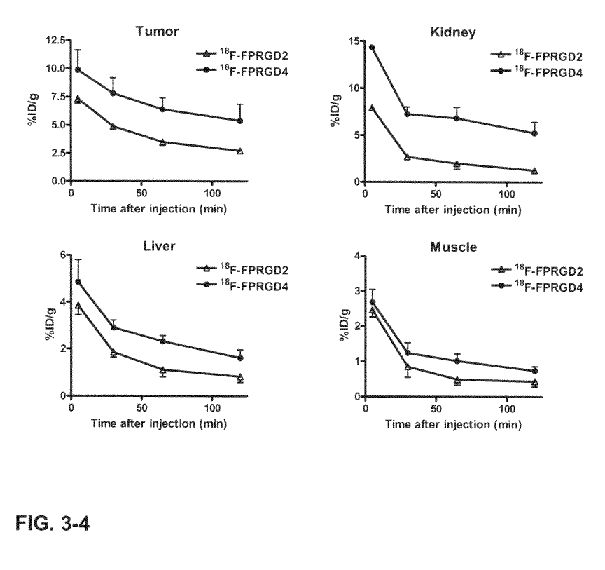

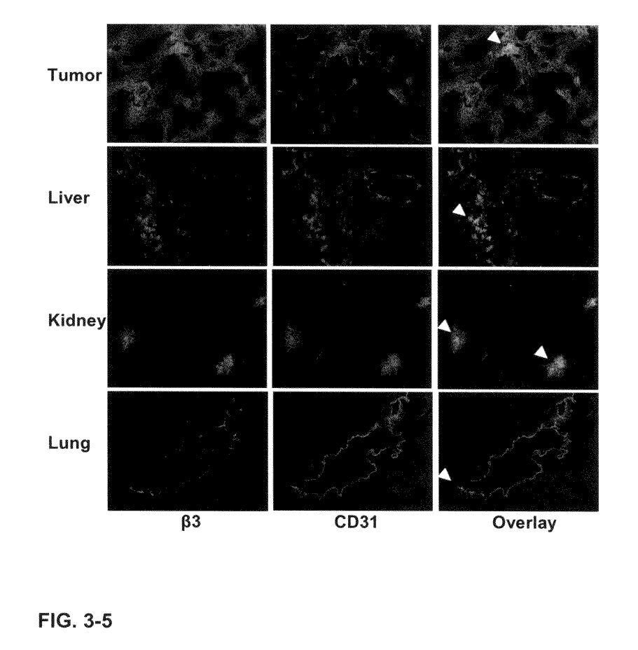

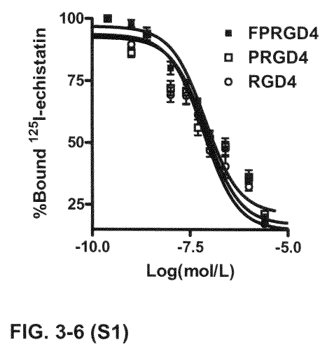

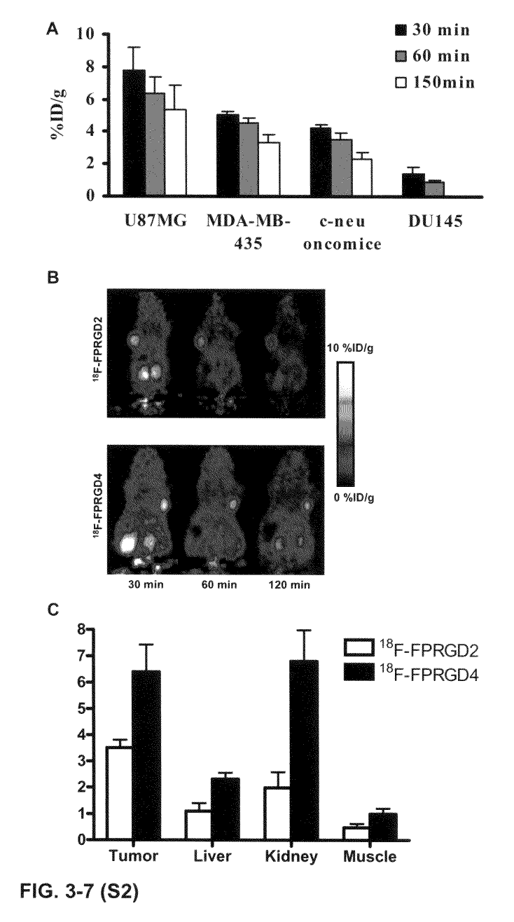

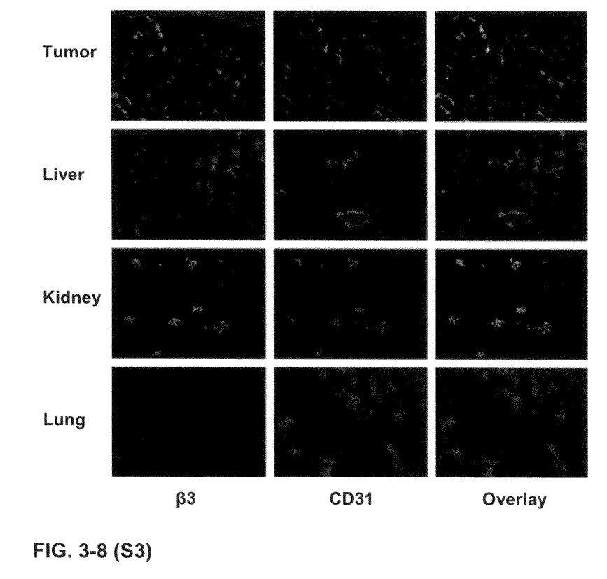

Dynamic microPET scans were performed on U87MG xenograft model and selected coronal images at different time points after injecting ¹⁸F-FPRGD2 were shown in FIG. 2-2a. High tumor uptake was observed as early as 5 min after injection. The U87MG tumor uptake was 4.9±0.1, 3.4±0.3, and 2.7±0.1% ID/g at 30 min, 1 h, and 2 h p.i. respectively (n=3). Most activity in the non-targeted tissues and organs had been cleared by 1 h p.i. For example, the uptake values in the kidneys, liver, and lung were as low as 2.0±0.6, 1.1±0.3, and 0.5±0.2% ID/g, respectively at 1 h p.i. For direct visual comparison, representative serial microPET images of U87MG tumor mice after injection of ¹⁸F-FRGD2 were also shown (FIG. 2-2b). It can be seen that both tracers gave comparable imaging quality, indicating that the mini-PEG spacer did not significantly alter the tumor targeting efficacy in vivo. Because of the very low tracer uptake in most organs especially in the abdominal region, ¹⁸F-FPRGD2 is suitable for imaging integrin positive lesions in most areas except for the kidneys and the urinary bladder. Time-activity curves showed that this tracer excreted predominantly through the renal route (FIG. 2-3).

The integrin αᵥβ₃ specificity of ¹⁸F-FPRGD2 in vivo was confirmed by a blocking experiment where the tracer was co-injected with c(RGDyK) (10 mg/kg). AS can be seen from FIG. 2-2c, the U87MG tumor uptake in the presence of non-radiolabeled RGD peptide (0.5±0.2% ID/g) is significantly lower than that without RGD blocking (3.4±0.3% ID/g) (P<0.00). Similar as previously reported [13], the tracer cleared from the body significantly faster and the uptake in most organs (e.g. kidneys and liver) were also lower than those without c(RGDyK) blocking. Western blot and immunohistochemical staining also confirmed that these organs express low levels of integrin αᵥβ₃ (data not shown).

MicroPET Imaging of C-Neu Oncomice with ¹⁸F-FPRGD2

The c-neu oncomice, a spontaneous tumor model which is more clinically relevant than the U87MG xenograft model, was also injected with ¹⁸F-FPRGD2 and scanned in the microPET scanner (FIG. 2-2d). This spontaneous breast tumor has been well-established in the literature to be integrin αᵥβ₃-positive (Bioconjug Chem 2006;17:1294-1313, Bioconjug Chem 2004;15:235-241, Cancer Biother Radiopharm 2003;18:627-641 and Anticancer Res 2005;25:197-206, each of which is incorporated herein by reference for the corresponding discussion). The spontaneous tumor uptake at 30 min p.i. was 3.6±0.1% ID/g (n=2), slightly higher than the kidney uptake (3.1±0.5% ID/g). The non-specific uptake in all the other organs was at background level (<1.5% ID/g). The tumor uptake dropped to 2.4±0.1% ID/g at 1 h p.i. Successful imaging of this spontaneous tumor model suggests the usefulness of ¹⁸F-FPRGD2 in detecting integrin αᵥβ₃-positive lesions in the clinical settings.

### Comparison of ¹⁸F-FPRGD2 and ¹⁸F-FRGD2

The comparison of tumor and various organ uptake of ¹⁸F-FPRGD2 and ¹⁸F-FRGD2 is shown in FIG. 2-4. The uptake in the U87MG tumor was essentially the same indicating that the two tracers have similar integrin αᵥβ₃ binding affinity and targeting efficacy in vivo (FIG. 2-4a). The kidney uptake is lower for ¹⁸F-FPRGD2 (FIG. 2-4b), at 2.7±0.2, 2.0±0.2, and 1.3±0.2% ID/g at 30 min, 1 h, and 2 h p.i. respectively. While for ¹⁸F-FRGD2, the kidney uptake was 3.6±0.1, 3.0±0.2, and 2.8±0.3% ID/g at 30 min, 1 h, and 2 h p.i. respectively. The liver uptake was similar for ¹⁸F-FPRGD2 and ¹⁸F-FRGD2 (FIG. 2-4c). The non-specific uptake in the muscle was slightly higher for ¹⁸F-FPRGD2 at early time points (e.g. 30 min p.i.) yet both were at a very low level (<0.5% ID/g, FIG. 2-4d) at 1 h p.i. Taken together, ¹⁸F-FPRGD2 had similar tumor, liver, and non-specific uptake as ¹⁸F-FRGD2, while the kidney uptake was appreciably lower.

### Metabolic Stability of ¹⁸F-FPRGD2

The metabolic stability of ¹⁸F-FPRGD2 was determined in mouse blood and urine samples and in the liver, kidneys, and U87MG tumor homogenates at 1 h p.i. (Table 1, Example 1). After centrifugation of the tissue homogenates, the majority of the injected radioactivity (75-95%) was in the supernatant (denoted as “extraction efficiency”), indicating successful recovery of the radiotracer from the mouse tissue. After passing the supernatant through C₁₈ Sep-Pak cartridges, most of the radioactivity was trapped and the non-retained fraction was less than 30%. After ACN elution, the radioactivity of each sample was injected onto an analytical HPLC and the HPLC chromatograms are shown in FIG. 2-5. The fraction of intact tracer (Rₜ: 15.8 min) was between 68% and 100% (Table 1, Example 1). A minor metabolite peak was found at about 13˜14 min for the blood and liver samples. No defluoridation was observed throughout the study. The metabolic stability of ¹⁸F-FPRGD2 was similar to ¹⁸F-FRGD2 (percentage of intact tracer was between 79% and 96%), demonstrating the incorporation of the mini-PEG spacer did not change the stability of the tracer in vivo.

### Discussion

We have labeled c(RGDyK) and E[c(RGDyK)]₂ with ¹⁸F using ¹⁸F-SFB as a prosthetic group (Mol Imaging 2004;3:96-104 J Nucl Med 2006;47:113-121, and Nucl Med Biol 2004;31:179-189, each of which is incorporated herein by reference for the corresponding discussion). ¹⁸F-FB-RGD had good tumor-to-blood and tumor-to-muscle ratios but also had rapid tumor washout and unfavorable hepatobiliary excretion. Because the natural mode of interactions between integrin αᵥβ₃ and RGD-containing proteins (e.g. vitronectin and fibronectin) involves multivalent binding sites, multimeric cyclic RGD peptides could improve the integrin αᵥβ₃ binding affinity thus leading to better targeting capability and higher cellular uptake through the integrin αᵥβ₃-dependent endocytosis pathway [2, 14, 15, 32] (Anti-Cancer Agents Med Chem 2006;6:407-428, Eur J Nucl Med Mol Imaging 2006;33, Suppl 13:54-63. Mol Pharm 2006;3:472-487, and J Am Chem Soc 2004;126:5730-5739, each of which is incorporated herein by reference for the corresponding discussion). Indeed, ¹⁸F-FRGD2 had two fold higher tumor uptake than the monomeric tracer ¹⁸F-FB-RGD. The dimeric RGD peptid tracer 18FRGD2 also allowed for quantification of the integrin αᵥβ₃ expression level in vivo, through either graphical analysis of dynamic PET scans (Logan plot) or the tumor-to-background ratio at 1 h p.i. when most of the nonspecific binding had been cleared. This property along with the excellent imaging quality and the favorable in vivo kinetics deserves clinical investigation in cancer patients. Unfortunately, the overall radiolabeling yield of ¹⁸F-FRGD2 was rather low. We believe that the low yield might be attributed to the steric hindrance and the low reactivity of the glutamate a-amino group (pKa: 9.47). In order to increase the overall radiolabeling yield and facilitate clinical translation, a mini-PEG spacer (three ethylene oxide units) was inserted between α-amine of the glutamate in E[c(RGDyK)]₂ and ¹⁸F-SFB.

It has been well established that PEG is a suitable polymer for the covalent modification of molecules for many pharmaceutical applications. Based on our previous reports where PEGylated (MW 3,400) RGD peptides were labeled with different isotopes, long PEG chain did improve the pharmacokinetics but at the same time also reduced the receptor binding affinity. Another concern of PEGylation is the heterogeneity of the resulting PEGylated compounds. Long-chain PEGs are mixtures of a broad range of different molecular weight compounds and polydispersity can create many problems in the characterization and quality control of the PEGylated compound. Reproducible production of PEGylated radiopharmaceuticals is quite difficult and is not amenable for clinical translation. We thus decided to use the mini-PEG spacer with definite molecular structure instead of the long polymeric PEG linker, aiming to minimize the PEGylation effect on the receptor binding affinity, imaging quality, tumor uptake, and in vivo kinetics.

To achieve optimal radiolabeling yield we tested different reaction conditions (solvent, temperature, pH, ¹⁸F-SFB/peptide ratio, reaction time, etc.). In our previous studies, the reaction between ¹⁸F-SFB and E[c(RGDyK)]₂ was carried out in borate buffer (pH 8.5). Because of hydrolysis, there are several side products (¹⁸F-FB and partially hydrolyzed species) that have similar HPLC retention time as the desired product ¹⁸F-FRGD2. The peaks of ¹⁸F-FRGD2 and ¹⁸F-FPRGD2 are both very close to ¹⁸F-FB, which makes the HPLC purification of the desired product quite difficult. In this study, we found that in anhydrous organic solvent (DMSO), the decay-corrected yield of ¹⁸F-FPRGD2 based on ¹⁸F-SFB was over 60%. The yield of ¹⁸F-FRGD2 under the same condition was significantly lower.

Comparison of the PET imaging results for ¹⁸F-FPRGD2 and ¹⁸F-FRGD2 revealed that ¹⁸F-FPRGD2 had comparable tumor uptake and non-specific muscle uptake, while the kidney uptake is appreciably lower. The residence time for kidneys (calculated based on the serial PET imaging data) is 0.016 h and 0.029 h for ¹⁸F-FPRGD2 and ¹⁸F-FRGD2, respectively. The shorter residence time is desirable as kidney is the only organ with appreciable tracer uptake and clearly the dose limiting organ. The uptake of ¹⁸F-FPRGD2 in the other major organs (e.g. liver and intestines) is at a very low level (less than 1.5% ID/g at 1 h p.i.) and will unlikely cause any adverse effects. Whether this is true for ¹⁸F-FPRGD2 remains to be tested in human patients.

In this Example, we used ¹⁸F-SFB for the peptide labeling via the amino group. To further improve the yield, other labeling strategies may also be explored. For ¹⁸F-labeling through the amino group at the N terminus or the lysine side chain, oxime formation and reductive amination using 4-¹⁸F-fluorobenzaldehyde (18F-FBA) (J Nucl Med 2004;45:892-902 and Clin Cancer Res 2004;10:3593-3606, each of which is incorporated herein by reference for the corresponding discussion), imidation reaction using 3-¹⁸F-fluoro-5-nitrobenzimidate (¹⁸F-FNB) (¹⁸F-FPB) (J Nucl Med 1987;28:462-470, which is incorporated herein by reference for the corresponding discussion), photochemical conjugation using 4-azidophenacyl ¹⁸F-fluoride (¹⁸F-APF) (Nucl Med Biol 1996;23:365-372, which is incorporated herein by reference for the corresponding discussion), and alkylation reactions using 4-¹⁸F-fluorophenacyl bromide (¹⁸F-FPB) (J Nucl Med 1987;28:462-470, which is incorporated herein by reference for the corresponding discussion) have been reported earlier. ¹⁸F-labeling of peptide or protein via the carboxylic acid group at the C terminus or glutamic/aspartic acid side chain is less common and only a few reports exist (Bioconjug Chem 1992;3:432-470, which is incorporated herein by reference for the corresponding discussion). We have previously reported the thiol-reactive synthon for thiolated RGD peptide labeling (J Nucl Med 2006;47:1172-1180, which is incorporated herein by reference for the corresponding discussion). Although the reaction between the thiol-reactive synthon and the thiolated RGD peptides was virtually quantitative, the synthesis of the thiol-reactive synthon required significant effort and time. Recently, click chemistry has been applied for ¹⁸F-labeling (Tetrahedron Lett 2006;47:6681-6684, which is incorporated herein by reference for the corresponding discussion). Although the labeling of model peptides was accomplished in good yield, there has been no in vivo PET data reported. Microfluidics has also been utilized for rapid and efficient synthesis of radiotracers and such strategy may be explored in the future for ¹⁸F-SFB/peptide coupling to minimize the amount of solvent used and further increase the overall yield (Science 2005;310:1793-1796, which is incorporated herein by reference for the corresponding discussion).

### Conclusion

¹⁸F-FPRGD2 had high activity accumulation in αᵥβ₃-integrin rich U87MG tumors and spontaneous mammary carcinnoma after injection. Excellent image quality, high integrin αᵥβ₃ binding affinity/specificity, and good metabolic stability comparable to ¹⁸F-FRGD2 were all maintained after incorporation of the mini-PEG spacer (11-amino-3,6,9-trioxaundecanoic acid). In addition, the radiolabeling yield was significantly improved and the renal uptake were significantly lowered for ¹⁸F-FPRGD2 than those of ¹⁸F-FRGD2, all of which makes ¹⁸F-FPRGD2 suitable for clinical PET applications.

TABLE 1
Example 1. Extraction efficiency, elution efficiency, and HPLC analysis of
soluble fractions of tissue homogenates at 1 h post-injection of
¹⁸F-FPRGD2 (“ND” denotes “not determined”).

| | | | | | |
|---|---|---|---|---|---|
| Fraction | Blood | Urine | Liver | Kidney | U87MG |

Extraction efficiency (%)

| | | | | | |
|---|---|---|---|---|---|
| Unsoluble fraction | 5.2 | ND | 23.3 | 21.8 | 24.4 |
| Soluble fraction | 94.8 | ND | 76.7 | 78.2 | 75.6 |

Elution efficiency (%)

| | | | | | |
|---|---|---|---|---|---|
| Nonretained fraction | 2.4 | 1.2 | 23.7 | 12.6 | 28.4 |
| Wash water | 1.2 | 0.2 | 4.3 | 2.0 | 4.3 |
| Acetonitrile eluent | 96.4 | 98.6 | 72.0 | 85.4 | 67.4 |

HPLC analysis (%)

| | | | | | |
|---|---|---|---|---|---|
| Intact tracer | 74.2 | 99.6 | 68.8 | 97.1 | 96.6 |

### Example 2

### Introduction

In vivo imaging of αᵥβ₃ expression has important diagnostic and therapeutic applications. Multimeric cyclic RGD peptides are capable of improving the integrin αᵥβ₃ binding affinity due to the polyvalency effect. In this study, we labeled PEGylated tetrameric RGD peptide NH₂-mini-PEG-E{E[c(RGDyK)]₂}₂ with ¹⁸F in reasonable yield and compared the tumor targeting efficacy and in vivo kinetics of the RGD tetramer with those of the RGD dimer analogs. Here we report the first example of ¹⁸F-labeled tetrameric RGD peptide for positron emission tomography (PET) imaging of αᵥβ₃ expression in both xenograft and spontaneous tumor models.

The tetrameric RGD peptide E{E[c(RGDyK)]₂}₂ was derived with amino-3,6,9-trioxaundecanoic acid (mini-PEG) linker through the glutamate a-amino group. NH₂-mini-PEG-E{E[c(RGDyK)]₂}₂ (PRGD4) was labeled with ¹⁸F via the N-succinimidyl-4-¹⁸F-fluorobenzoate (¹⁸F-SFB) prosthetic group. The receptor binding characteristics of the tetrameric RGD peptide tracer ¹⁸F-FPRGD4 was evaluated in vitro by cell binding assay and in vivo by quantitative microPET imaging studies. The decay-corrected radiochemical yield for ¹⁸F-FPRGD4 was about 15% with a total reaction time of 180 min starting from ¹⁸F-F⁻. The PEGylation had minimal effect on integrin binding affinity of the RGD peptide. ¹⁸F-FPRGD4 has significantly higher tumor uptake compared with monomeric and dimeric RGD peptide tracer analogs. The prominent uptake and retention of ¹⁸F-FPRGD4 in the kidneys is likely attributed to both renal clearance pathway of this hydrophilic radiotracer and integrin αᵥβ₃ positiveness of rodent kidneys. The receptor specificity of ¹⁸F-FPRGD4 in vivo was confirmed by effective blocking of the uptakes in both tumors and normal organs/tissues with excess c(RGDyK).

The tetrameric RGD peptide tracer ¹⁸F-FPRGD4 possessing high integrin binding affinity and favorable biokinetics is a promising tracer for PET imaging of integrin αᵥβ₃ expression in cancer and other angiogenesis related diseases.

### Materials and Methods

All chemicals obtained commercially were of analytical grade and used without further purification. No-carrier-added ¹⁸F-F⁻ was obtained from in-house PETtrace cyclotron (GE Healthcare). Reversed-phase extraction C-18 Sep-Pak cartridges were obtained from Waters and were pretreated with ethanol and water before use. The syringe filter and polyethersulfone membranes (pore size, 0.22 μm; diameter, 13 mm) were obtained from Nalge Nunc International. ¹²⁵I-Echistatin, labeled by the lactoperoxidasemethod to a specific activity of 74,000 GBq/mmol (2,000 Ci/mmol), was purchased from GE Healthcare. Analytical as well as semi-preparative reversed-phase high-performance liquid chromatography (RP-HPLC) was performed on a Dionex 680 chromatography system with a UVD 170U absorbance detector and model 105S single-channel radiation detector (Carroll & Ramsey Associates). The recorded data were processed using Chromeleon version 6.50 software. Isolation of peptides and ¹⁸F-labeled peptides was performed using a Vydac protein and peptide column (218TP510; 5 μm, 250×10 mm). The flow was set at 5 mL/min using a gradient system starting from 95% solvent A (0.1% trifluoroacetic acid [TFA] in water) and 5% solvent B (0.1% TFA in acetonitrile [ACN]) (0-2 min) and ramped to 35% solvent A and 65% solvent B at 32 min. The analytical HPLC was performed using the same gradient system, but with a Vydac column (218TP54, 5 μm, 250×4.6 mm) and flow of 1 mL/min. The ultraviolet (UV) absorbance was monitored at 218 nm and the identification of the peptides was confirmed based on the UV spectrum acquired using a PDA detector.

Preparation of NH₂-mini-PEG-CO-E{E[c(RGDyK)]₂}₂ (PRGD4)

The E{E[c(RGDyK)]₂}₂ (denoted as RGD4) was prepared from cyclic RGD dimer E[c(RGDyK)]₂ according to our previously reported procedure (J Nucl Med. 2005;46:1707-1718, which is incorporated herein by reference for the corresponding discussion). To a solution of Boc-11-amino-3,6,9-trioxaundecanoic acid (Boc-NH-mini-PEG-COOH, 40 mg, 0.13 mmol) and 20 μL DIPEA in ACN was added O-(N-Succinimidyl)-1,1,3,3-tetramethyl- uronium tetrafluoroborate (TSTU, 27 mg, 0.09 mmol). The reaction mixture was stirred at room temperature for 0.5 h and then added to E{E[c(RGDyK)]₂}₂ (10 mg, 3.6 μmol) in N,N′-dimethylformamide (DMF). The reaction was stirred at room temperature for another 2 h and the desired product Boc-NH-mini-PEG-CO-E{E[c(RGDyK)]₂}₂ was isolated by semi-preparative HPLC. The collected fractions were combined and lyophilized to give a fluffy white powder (60% yield). The Boc-group was readily removed by treating Boc-NH-mini-PEG-CO-E{E[c(RGDyK)]₂}₂ with anhydrous TFA for 5 min at room temperature. The crude product was purified by HPLC. The collected fractions were combined and lyophilized to afford NH₂-mini-PEG-CO-E{E[c(RGDyK)]₂}₂ (denoted as PRGD4) as a white powder (90%). Analytical HPLC (Rt=13 min) and mass spectrometry (MALDI-TOF-MS: m/z 3001.0 for [MH]⁺ (C₁₃₁H₁₉₄N₄₀O₄₂, calculated molecular weight [MW] 3001.1)) confirmed the identity of the purified product.

Preparation of FB-NH-mini-PEG-CO-E{E[c(RGDyK)]₂}₂ (FPRGD4)

N-succinimidyl-4-fluorobenzoate (SFB) (4 mg, 16.8 μmol) and PRGD4 (2 mg, 0.67 μmol) were mixed in 0.05 M borate buffer (pH 8.5) at room temperature. After 2 h, the desired product FB-NH-mini-PEG-CO-E{E[c(RGDyK)]₂}₂ (denoted as FPRGD4) was isolated by semi-preparative HPLC in 65% yield. Analytical HPLC (Rₜ=15.7 min) and mass spectrometry (MALDI-TOF-MS: m/z 3123.4 for [MH]⁺ (C₁₃₈H₁₉₇FN₄₀O₄₃, calculated [MW] 3123.3) analyses confirmed product identification.

### Radiochemistry

N-Succinimidyl-4-¹⁸F-fluorobenzoate (¹⁸F-SFB) was synthesized according to our previously reported procedure (Nucl Med Biol. 2004;31:179-189, which is incorporated herein by reference for the corresponding discussion). Recently, we adapted the procedure into a commercially available synthesis module (GE TRACERIab FX_{FN}). The purified ¹⁸F-SFB was rotary evaporated to dryness, reconstituted in dimethyl sulfoxide (DMSO, 200 μL), and added to a DMSO solution of PRGD4 (300 μg, 0.1 μmol) with DIPEA (20 μL). The peptide mixture was incubated at 60° C. for 30 min. After dilution with 700 μL of water with 1% TFA, the mixture was purified by semi-preparative HPLC. The desired fractions containing ¹⁸F-FPRGD4 (FIG. 3-1) were combined and rotary evaporated to remove the solvent. ¹⁸F-FPRGD4 was then formulated in normal saline and passed through a 0.22-μm Millipore filter into a sterile multidose vial for in vivo experiments.

### Octanol-Water Partition Coefficient

Approximately 111 kBq of ¹⁸F-FPRGD4 in 500 μL of PBS (pH 7.4) were added to 500 μL of octanol in an Eppendorf microcentrifuge tube. The mixture was vigorously vortexed for 1 min at room temperature. After centrifugation at 12,500 rpm for 5 min in an Eppendorf microcentrifuge (model 5415R, Brinkman), 200 μL aliquots of both layers were measured using a γ-counter (Packard Instruments). The experiment was carried out in triplicates.

### Cell Line and Animal Model

Animal procedures were performed according to a protocol approved by the Stanford University Institutional Animal Care and Use Committee. The U87MG tumor model was generated by subcutaneous injection of 5×10⁶ cells into the front flank of female athymic nude mice (Harlan, Indianapolis, Ind.). The MDA-MB-435 tumor model was established by orthotopic injection of 5×10⁶ cells into the left mammary fat pad of female athymic nude mice. The DU145 tumor model was established by subcutaneous injection of 5×10⁶ cells into the left front flank of male athymic nude mice. The mice were subjected to microPET studies when the tumor volume reached 100-300 mm³ (3-4 weeks after inoculation). The c-neu oncomouse (Charles River Laboratories, Charles River, Canada) is a spontaneous tumor-bearing model that carries an activated c-neu oncogene driven by a mouse mammary tumor virus (MMTV) promoter (Cell. 1988;54:105-115, which is incorporated herein by reference for the corresponding discussion). Transgenic mice uniformly expressing the MMTV/c-neu gene develop mammary adenocarcinomas between 4 and 8 months postpartum that involve the entire epithelium in each gland. These mice were subjected to microPET scans at about 8 months old and the tumor volume was about 300-500 mm³.

### Cell Integrin Receptor-Binding Assay

In vitro integrin αᵥβ₃-binding affinities and specificities of RGD4, PRGD4 and FPRGD4 were assessed via displacement cell binding assays using ¹²⁵I-echistatin as the integrin αᵥβ₃-specific radioligand. Experiments were performed on U87MG human glioblastoma cells by the method previously described (J Nucl Med. 2005;46:1707-1718 J Nucl Med. 2006;47:1172-1180, which is incorporated herein by reference for the corresponding discussion). The best-fit 50% inhibitory concentration (IC₅₀) values for the U87 MG cells were calculated by fitting the data with nonlinear regression using Graph-Pad Prism (GraphPad Software, Inc.). Experiments were performed with triplicate samples.

microPET Imaging Studies

PET scans and image analysis were performed using a microPET R4 rodent model scanner (Siemens Medical Solutions) as previously reported. For U87MG tumor model, mice (n=3) were tail-vein injected with about 3.7 MBq (100 μCi) of ¹⁸F-FPRGD4 under isoflurane anesthesia and then subjected to a 30-min dynamic scan (1×30 s, 4×1 min, 1×1.5 min, 4×2 min, 1×2.5 min, 4×3 min, total of 15 frames) starting from 1 min p.i. Five min static PET images were also acquired at 1, 2, and 3 h p.i. The images were reconstructed by 2-dimensional ordered-subsets expectation maximum (OSEM) algorithm. No attenuation or scatter correction was applied. For receptor-blocking experiment, a U87MG tumor mouse was co-injected with 10 mg/kg mouse body weight of c(RGDyK) and 3.7 MBq of ¹⁸F-FPRGD4. The 5-min static PET scans was then acquired at 30 min and 1 h p.i. Multiple time point static scans were also obtained for orthotopic MDA-MB-435, c-neu oncomouse, and subcutaneous DU145 tumor models after tail-vein injected with 3.7 MBq of ¹⁸F-FPRGD4.

For each microPET scan, regions of interest (ROIs) were drawn over the tumor, normal tissue, and major organs by using vendor software (ASI Pro 5.2.4.0) on decay-corrected whole-body coronal images. The maximum radioactivity concentration (accumulation) within a tumor or an organ was obtained from mean pixel values within the multiple ROI volume, which were converted to counts/mL/min by using a conversion factor. Assuming a tissue density of 1 g/mL, the ROIs were converted to counts/g/min and then divided by the administered activity to obtain an imaging ROI-derived % ID/g.

Immunofluorescence Staining of c-Neu Oncomice

Frozen tumor and organ tissue slices (5 μm thickness) were fixed with ice cold acetone for 10 min and dried in air for 30 min. The slices were rinsed with PBS for 3 min and blocked with 10% goat serum for 30 min at room temperature. The slices were incubated with rat anti-mouse CD31 antibody (1:100, BD Biosciences, San Jose, Calif.) and hamster anti-β₃ antibody (1:100, BD Biosciences) for 3 h at room temperature, then visualized with Cy3-conjugated goat anti-hamster and FITC-conjugated goat anti-rat secondary antibody (1:200, Jackson ImmunoResearch Laboratories, Inc., West Grove, Pa.).

### Statistical Analysis

Quantitative data was expressed as mean±SD. Means were compared using One-way ANOVA and student's t-test. P values<0.05 were considered statistically significant.

### Results

### Chemistry and Radiochemistry

The synthesis of RGD tetramer was performed through an active ester method by coupling Boc-Glu(OSu)₂ with dimeric RGD peptides followed by TFA deprotection. Boc-NH-mini-PEG-COOH was activated with TSTU/DIPEA, and then conjugated with the amino group of tetrameric RGD peptide under a slightly basic condition. After TFA deprotection, PRGD4 was obtained as fluffy white powder. The total synthesis time for ¹⁸F-SFB was about 100 min and the decay-corrected yield was 67±11% (n=10) using the modified GE synthetic module (TRACERIab FX_{FN}). The decay-corrected radiochemical yield of ¹⁸F-FPRGD4 based on ¹⁸F-SFB was 22.0±0.8% (n=4). The radiochemical purity of ¹⁸F-FPRGD4 was >99% according to analytical HPLC. The specific radioactivity of ¹⁸F-FPRGD4 was determined to be about 100-200 TBq/mmol based on the labeling agent ¹⁸F-SFB, since the unlabeled PRGD4 was efficiently separated from the product. Starting from ¹⁸F-F⁻, the total synthesis time of ¹⁸F-FPRGD4 including the final HPLC purification was about 180 min and the overall decay-corrected yield was 15±4%. In comparison, the yield of coupling E{E[c(RGDyK)]₂}₂ with ¹⁸F-SFB was less than 2% (data not shown). The octanol/water partition coefficient (logP) for ¹⁸F-FPRGD4 was −2.67±0.22, which was slightly lower than ¹⁸F-FRGD2 (−2.10±0.03) and ¹⁸F-FPRGD2 (−2.28+0.05) (Eur J Nucl Med Mol Imaging. 2007, which is incorporated herein by reference for the corresponding discussion).

### In Vitro Cell Integrin Receptor-Binding Assay

The receptor-binding affinity of RGD4, PRGD4 and FPRGD4 was determined by performing competitive displacement studies with ¹²⁵I-echistatin. All peptides inhibited the binding of ¹²⁵I-echistatin (integrin αᵥβ₃ specific) to U87MG cells in a concentration dependent manner. The IC₅₀ values for RGD4, PRGD4 and FPRGD4 were 39.1±5.5, 46.5±5.3 and 37.7±7.0 nM, respectively (n=3) (FIG. 3-6). The comparable IC₅₀ values of all three compounds suggest that the insertion of miniPEG linker and fluorobenzoyl coupling had minimal effect on the receptor binding affinity.

microPET Imaging of ¹⁸F-FPRGD4 on Tumor-Bearing Mice

Dynamic microPET scans were performed on U87 MG xenograft model and selected coronal images at different time points after injection of ¹⁸F-FPRGD4 were shown in FIG. 3-2(A). The tumor was clearly visible with high contrast to contralateral background as early as 5 min p.i. Quantitation of tumor and major organ activity accumulation in microPET scans was realized by measuring ROIs encompassing the entire organ in the coronal orientation. The U87MG tumor uptake of ¹⁸F-FPRGD4 was calculated to be 9.87±0.10, 7.80±0.14, 6.40±0.27, 5.39±0.14, and 4.82±0.22% ID/g at 5, 30, 60, 120 and 180 min p.i., respectively (n=3). The averaged time-activity curves (TACs) for the U87MG tumor, liver, kidneys, heart, lung, and muscle were shown in FIG. 3-3. ¹⁸F-FPRGD4 was cleared mainly through the kidneys. Some hepatic clearance was also observed.

Representative coronal microPET images of MDA-MB-435 tumor-bearing mice (n=3) at different times after tracer injection were showed in FIG. 3-2C. As the integrin expression level in MDA-MB-435 tumor is lower than U87MG, the tumor uptake of ¹⁸F-FPRGD4 in MDA-MB-435 tumor (5.07±0.18, 4.53±0.36, 3.38±0.48% ID/g at 30, 60, and 150 min p.i.) was also lower than that in U87MG tumor. No significant difference in normal organs and tissues was found between these two tumor models.

¹⁸F-FPRGD4 was also successful in visualizing a spontaneous murine mammary carcinoma model grown in c-neu oncomice (FIG. 3-2B) (Cancer Biother Radiopharm. 2003;18:627-641; Bioconjug Chem. 2006;17:1294-1313; Bioconjug Chem. 2004;15:235-241; and J Cardiovasc Pharmacol. 2005;45:109-113, each of which are incorporated herein by reference for the corresponding discussion). The tumor uptakes were found to be 4.22±0.18, 3.56±0.34, and 2.36±0.40% ID/g at 30, 60, and 150 min, respectively (n=3). These values are slightly lower than those in MDA-MB-435 human breast cancer tumors grown in nude mice. No significant difference was found in major organs and tissues between the spontaneous tumor model of Balb/C strain and the xenograft models of nude mice strain.

FIG. 3-7(A) illustrate the comparison between the uptakes of ¹⁸F-FPRGD4 in different tumors and kidneys over time for tumor-bearing mice. Data was derived from multiple time-point microPET study. ROIs are shown as the % ID/g±SD (n=3). FIG. 3-8(B) illustrates the direct visual comparison of microPET images of U87MG tumor-bearing mice after intravenous injection of ¹⁸F-FPRGD4 and ¹⁸F-FPRGD2. FIG. 3-8(C) illustrates a comparison of biodistribution (based on PET, 60 min p.i.) results for ¹⁸F-FPRGD4 and ¹⁸F-FPRGD2 on U87MG tumor-bearing mice.

We also tested ¹⁸F-FPRGD4 in an integrin negative DU145 tumor model (n=3). As can be seen from FIG. 3-2D, only slightly higher than contralateral muscle background signal was detected in DU145 tumor (1.44±0.34 and 0.93±0.13% ID/g at 30 and 60 min p.i.). These values were significantly lower than in all other three integrin-expressing tumor models (P<0.001). The tumor uptake followed the trend of U87MG>MDA-MB-435>c-neu>DU145 (FIG. 3-8), which is consistent with the integrin αᵥβ₃ expression pattern (quantified by SDS-PAGE/autoradiography) (Eur J Nucl Med Mol Imaging. 2007, which is incorporated herein by reference for the corresponding discussion), which is incorporated herein by reference for the corresponding discussion) in these tumor models (data not shown).

The integrin αᵥβ₃ specificity of ¹⁸F-FPRGD4 in vivo was also confirmed by a blocking experiment. Representative coronal images of U87MG tumor mice after injection of ¹⁸F-FPRGD4 in the presence of blocking dose of c(RGDyK) (10 mg/kg of mouse body weight) were illustrated in FIG. 3-2E. More than 80% of the uptake in the tumor was inhibited as compared with that in the tumor without blocking (FIG. 3-2A). Radioactivity accumulation in most other major organs and tissues was also significantly reduced in the presence of non-radioactive RGD peptide.

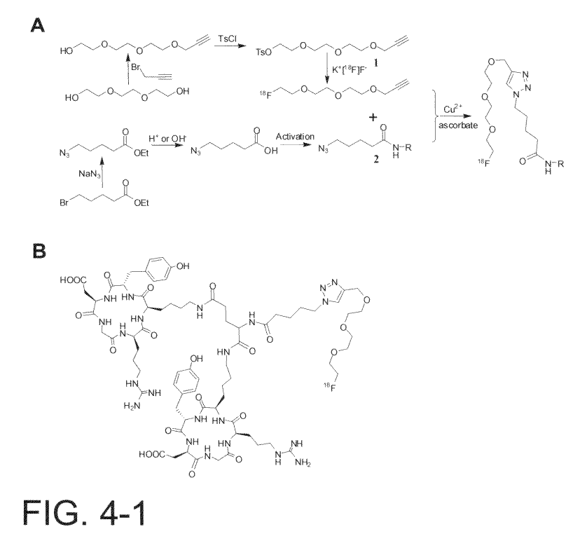

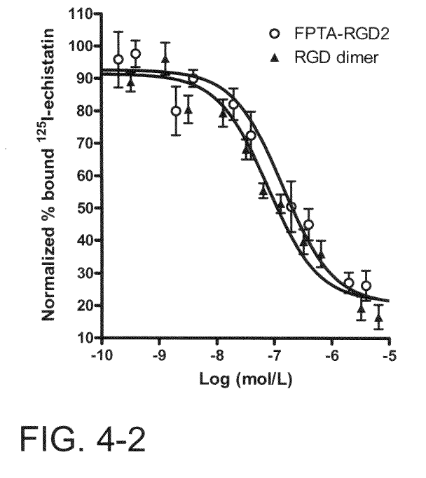

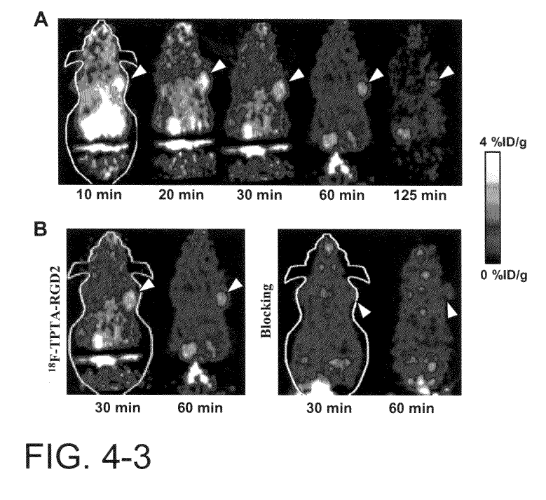

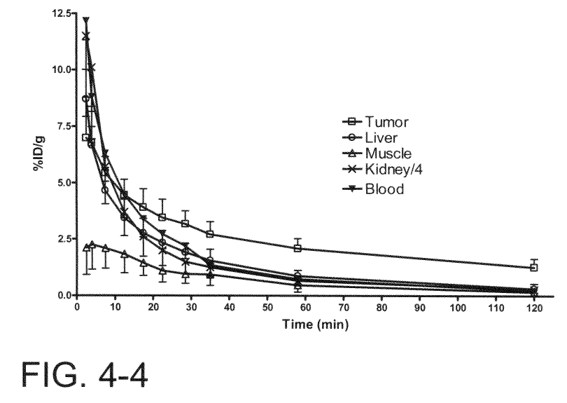

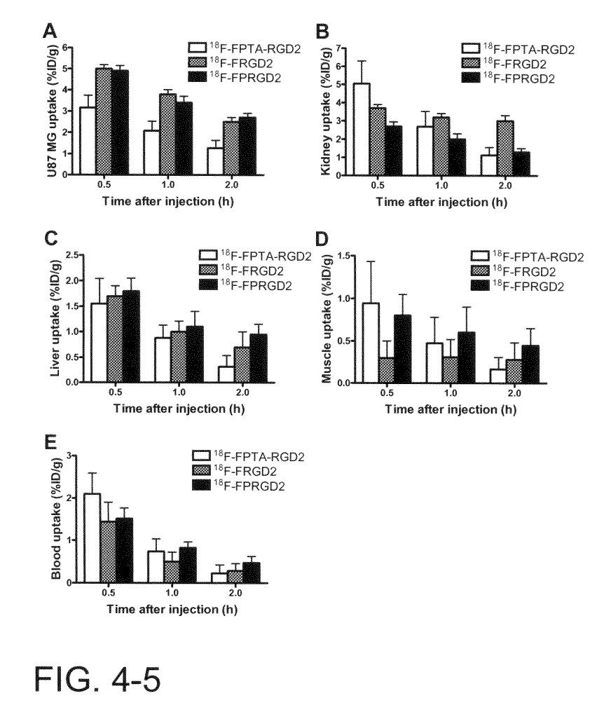

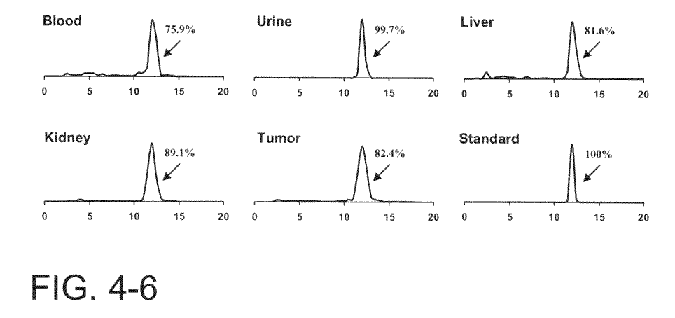

The tumor uptake and biodistribution of ¹⁸F-FPRGD4 derived from quantitative microPET imaging was compared with that of the dimeric analog ¹⁸F-FPRGD2 in the same U87MG tumor model (Eur J Nucl Med Mol Imaging. 2007, which is incorporated herein by reference for the corresponding discussion). As shown in FIG. 3-4, the uptake of ¹⁸F-FPRGD4 in U87MG tumor was significantly higher than that of ¹⁸F-FPRGD2 at all time points examined (P<0.001). ¹⁸F-FPRGD4 also showed higher uptake than ¹⁸F-FPRGD2 in the liver, kidneys (P<0.05). The initial muscle uptake of ¹⁸F-FPRGD4 was higher than ¹⁸F-FPRGD2 (P<0.05), but the difference was diminished at late time points (P>0.05).

Immunofluorescence Staining of c-Neu Oncomice

The frozen tumor, liver, kidney and lung tissue slices harvested from c-neu oncomice were stained for CD31 and mouse β₃-integrin. As can be seen in FIG. 3-5, β₃-integrin was expressed in both tumor cells and endothelial cells of the murine mammary carcinoma as most of the CD31 positive vessels were also β₃ positive. Integrin β₃ was also detected in the liver, lung and kidneys. In particular, strong staining of integrin β₃ was found in the glomerulus, which might be partially responsible for high renal uptake of ¹⁸F-FPRGD4. Similar integrin expression pattern was also seen in athymic nude mice (FIG. 3-8).

### Discussion

A variety of radiolabeled RGD peptides have been evaluated for tumor localization and therapy. However, most of the monomeric RGD peptide-based tracers developed so far have fast blood clearance accompanied by relatively low tumor uptake and rapid tumor washout, presumably due to the suboptimal receptor-binding affinity/selectivity and inadequate contact with the binding pocket located in the extracellular segment of integrin αᵥβ₃. The natural functional mode of integrin binding involves multivalent interactions, which could provide not only more effective binding molecules but also systems that could improve the cell targeting and promote cellular uptake. Thus, we and others have applied polyvalency principle to develop dimeric and multimeric RGD peptides. We have labeled c(RGDyK) and E[c(RGDyK)]₂ with ¹⁸F using ¹⁸F-SFB as a prosthetic group. ¹⁸F-FB-RGD (¹⁸F-FRGD) had good tumor/muscle ratio but rapid tumor washout and unfavorable hepatobiliary excretion, limiting its potential applications for imaging αᵥ-integrin positive tumors in the lower abdomen area. In contrast, the dimeric RGD peptide tracer ¹⁸F-FRGD2 had significantly higher tumor uptake and prolonged tumor retention than ¹⁸F-FRGD because of the synergistic effect of bivalency and improved pharmacokinetics (J Nucl Med. 2006;47:1172-1180 and J Nucl Med. 2004;45:1776-1783, each of which is incorporated herein by reference for the corresponding discussion). It is logical to assume that tetrameric RGD peptide tracer would be superior to the dimeric and monomeric peptide analogs due to the enhanced receptor binding caused by polyvalency effect. However, the labeling yield of ¹⁸F-FRGD4 was not satisfactory, owing in part to the bulk of the four cyclic pentapeptides and the prosthetic group N-succinimidyl-4-¹⁸F-fluorobenzoate (¹⁸F-SFB). The glutamate a-amine group has a pKa of 9.47, which is also less reactive than the a-amino group on the lysine side chain (pKa=8.95) usually used for ¹⁸F labeling of peptides.

In order to overcome the problem of low labeling yield, we wanted to insert a poly(ethylene glycol) (PEG) linker between the RGD tetramer and the prosthetic ¹⁸F-labeling group. PEG moieties are inert, long-chain amphiphilic molecules produced by linking repeating units of ethylene oxide. PEGylation can decrease clearance, retain biological activity, obtain a stable linkage, and enhance water solubility without significantly altering bioavailability. Moreover, polyethylene glycol spacers are nontoxic and unreactive. PEGylation has been widely used for improving the in vivo kinetics of various pharmaceuticals. Based on the previous studies, we found that PEGylated (MW 3,400) RGD peptides had lower integrin binding affinity than non-PEGylated ones. Moreover, long-chain PEGs are mixtures of a broad range of different molecular weight compounds. Polydispersity of PEG complicates the characterization and quality control of the PEGylated compounds. In contrast, a miniPEG spacer with definite molecular structure has been successfully used to reduce the spatial hindrance and improve the labeling yield for the dimeric RGD peptide (Eur J Nucl Med Mol Imaging. 2007, which is incorporated herein by reference for the corresponding discussion). It was also found that this PEGylation had minimal effect on the receptor binding affinity, imaging quality, tumor uptake, and in vivo kinetics of dimeric RGD peptide E[c(RGDyK)]₂. We thus decided to employ this strategy to make fluorine-18 labeled tetrameric RGD peptide. Indeed, the coupling yield between PRGD4 and ¹⁸F-SFB was over 20% while the same reaction between RGD4 and ¹⁸F-SFB was less than 2%. PRGD4 and FPRGD4 had similar integrin binding affinity as RGD4, demonstrating that miniPEGylation had a minimal effect on the integrin affinity of this RGD tetramer.

The imaging quality of ¹⁸F-FPRGD4 was tested in a U87MG human glioblastoma xenograft model, which has been well established to have high integrin expression. Compared with ¹⁸F-FPRGD2, the tumor uptake of ¹⁸F-FPRGD4 was more than 50% higher at all time points in U87 MG xenograft model (FIG. 3-4). The initial high tumor uptake might be mainly attributed to the high integrin affinity of ¹⁸F-FPRGD4, although other factors such as molecular weight, hydrophilicity, and circulation half-life may also affect the tumor accumulation and retention. No significant difference was observed in the tumor wash-out rate of ¹⁸F-FPRGD4 and ¹⁸F-FPRGD2. The increased uptake of ¹⁸F-FPRGD4 than ¹⁸F-FPRGD2 in the liver and kidneys may be due to the increased molecular size and some integrin expression in these organs. Overall, ¹⁸F-FPRGD4 had significantly higher tumor uptake than, and comparable tumor/liver and tumor/muscle ratios (P>0.1) with ¹⁸F-FPRGD2. A similar pattern was also found for ⁶⁴Cu labeled RGD peptides (J Nucl Med. 2005;46:1707-1718, which is incorporated herein by reference for the corresponding discussion).

In the blocking experiment, non-radioactive RGD peptide inhibited the uptake of ¹⁸F-FPRGD4 not only in U87MG tumor but also in several major organs (FIG. 3-2E). The biodistribution of ¹⁸F-FPRGD4 (FIG. 3-3 and FIG. 3-4) showed initial rapid clearance of activity in the liver and kidney but then reached a plateau. These phenomena suggest that some normal organs and tissues may also be integrin positive, although to a less extent, as confirmed by immunohistochemistry. Immunohistopathology showed strong positive staining of the endothelial cells of the small glomeruli vessels in the kidneys and weak staining in the branches of the hepatic portal vein. However, whether the higher renal uptake and retention of ¹⁸F-FPRGD4 is integrin αᵥβ₃ mediated is yet to be tested. Integrins play important roles in renal development and integrin αᵥβ₃, in particular, has been identified in many parts of the developing kidney. Rodent kidneys are constantly under development and thus high integrin expression in the glomeruli while adult human kidneys are more developed and thus less integrin expression. Thus, the relatively high renal uptake of ¹⁸F-FPRGD4 in mouse models may not be the same as in human adults if it mainly caused by integrin αᵥβ₃.

In this Example, we inserted a mini-PEG linker to improve the labeling yield between ¹⁸F-SFB and miniPEGylated RGD tetramer. The coupling yield of slightly higher than 20% based on ¹⁸F-SFB is still not satisfactory for routine clinical use. Furthermore, the synthesis of ¹⁸F-SFB synthon is quite time consuming. Other ¹⁸F-labeling strategies such as click chemistry (Tetrahedron Lett. 2006;47:6681-6684, which is incorporated herein by reference for the corresponding discussion), reductive amination (Int J Rad Appl Instrum [A]. 1992;43:1265-1274, which is incorporated herein by reference for the corresponding discussion), Michael addition for thiol-specific coupling (J Nucl Med. 2006;47:1172-1180, which is incorporated herein by reference for the corresponding discussion), and oxime formation (J Nucl Med. 2004;45:892-902, which is incorporated herein by reference for the corresponding discussion) may be utilized to simplify the labeling procedure and improve the labeling yield.

Although we have successfully demonstrated the specificity of ¹⁸F-FPRGD4 for high (U87MG), medium (MDA-MB-435 and c-neu), and low (DU145) integrin αᵥβ₃-expressing tumors, we did not determine whether the tumor/background contrast or the binding potential derived from Logan plot of the dynamic PET scans correlate well with the integrin expression level measured ex vivo by SDS-PAGE/autoradiography or Western blot. Due to the enhanced receptor binding, we found that the tetrameric RGD peptide tracer ¹⁸F-FPRGD4 showed significantly higher tumor uptake than its dimeric analog ¹⁸F-FPRGD2. However, the tumor/muscle and tumor/major-organ ratios were similar. Thereby, appropriate modification is needed to make it superior to the dimeric peptide analog ¹⁸F-FPRGD2 and the monomeric peptide analogs (¹⁸F-FRGD or ¹⁸F-Galacto-RGD). By replacing the mini-PEG linker with other pharmacokinetic modifiers, we may be able to modulate the overall molecular charge, hydrophilicity, and molecular size, thus possibly improving in vivo pharmacokinetics without compromising the tumor-targeting efficacy of the resulting radioconjugates. Moreover, the cost of tetrameric RGD peptides as compared to the dimeric and monomeric analogs cannot be ignored. More careful side-by-side comparisons among ¹⁸F-FPRGD4, ¹⁸F-FRGD2, and ¹⁸F-Galacto-RGD in human patients may be needed to assess the dosimetry and tumor targeting sensitivity/specificity and eventually identify the optimal RGD peptide tracer for PET imaging of integrin expression.

### Conclusion

A new tetrameric RGD peptide tracer ¹⁸F-FPRGD4 was designed and synthesized with good yield. Due to the polyvalency effect, this tracer showed high αᵥβ₃-integrin binding affinity and specificity both in vitro and in vivo. ¹⁸F-FPRGD4 had much higher tumor uptake (6.40±0.27% ID/g at 60 min p.i.) than the monomeric and dimeric RGD peptide analogs (3.80±0.10% ID/g for ¹⁸F-FRGD and 3.40±0.10% ID/g for ¹⁸F-FPRGD2 at 60 min p.i.). The microPET imaging studies performed in different tumor model suggest that ¹⁸F-FPRGD4 may have great potential as a clinical PET radiopharmaceutical for imaging tumor integrin expression. The mini-PEG spacer (11-amino-3,6,9-trioxaundecanoic acid) is a suitable chemical means to modify the tumor targeting ability and physiological behavior of the tetrameric RGD peptide and can improve the radiolabeling yield using ¹⁸F-SFB as a prosthetic group.

### Example 3

### Introduction

The cell adhesion molecule integrin αᵥβ₃ plays a key role in tumor angiogenesis and metastasis. A series of ¹⁸F-labeled RGD peptides have been developed for PET of integrin expression based on primary amine-reactive prosthetic groups. In this study we report the use of the Cu(I)-catalyzed Huisgen cycloaddition, also known as a ‘click reaction’, to label RGD peptides with ¹⁸F by forming 1,2,3-triazoles. Nucleophilic fluorination of a toluenesulfonic alkyne provided ¹⁸F-alkyne in high yield (non-decay-corrected yield: 65.0±1.9%, starting from the azeotropically-dried ¹⁸F-fluoride), which was then reacted with an RGD azide (non-decay-corrected yield: 52.0±8.3% within 45 min including HPLC-purification). The ¹⁸F-labeled peptide was subjected to microPET studies in murine xenograft models. Murine microPET experiments showed good tumor uptake (2.1±0.4% ID/g at 1 h postinjection (p.i.)) with rapid renal and hepatic clearance of ¹⁸F-fluoro-PEG-triazoles-RGD₂ (¹⁸F-FPTA-RGD2) in a subcutaneous U87MG glioblastoma xenograft model (kidney: 2.7±0.8% ID/g, liver: 1.9±0.4% ID/g at 1 h p.i.). Metabolic stability of the newly synthesized tracer was also analyzed (intact tracer ranging from 75-99% at 1 h p.i.). In brief, the new tracer ¹⁸F-FPTA-RGD2 was synthesized with high radiochemical yield and high specific activity. This tracer exhibited good tumor-targeting efficacy, relatively good metabolic stability, as well as favorable in vivo pharmacokinetics. This new ¹⁸F labeling method based on ‘click reaction’ may also be useful for radio-labeling of other biomolecules with azide group in high yield.

### Materials and Methods

All chemicals obtained commercially were of analytical grade and used without further purification. No-carrier-added ¹⁸F-F⁻ was obtained from a PETtrace cyclotron (GE Healthcare). Reversed-phase extraction C-18 Sep-Pak cartridges were obtained from Waters and were pretreated with ethanol and water before use. The syringe filter and polyethersulfone membranes (pore size, 0.22 μm; diameter, 13 mm) were obtained from Nalge Nunc International. ¹²⁵I-echistatin, labeled by the lactoperoxidasemethod to a specific activity of 74,000 GBq/mmol (2,000 Ci/mmol), was purchased from GE Healthcare. Analytical as well as semi-preparative reversed-phase high-performance liquid chromatography (RP-HPLC) was performed on a Dionex 680 chromatography system with a UVD 170U absorbance detector and model 105S single-channel radiation detector (Carroll & Ramsey Associates). The recorded data were processed using Chromeleon version 6.50 software. Isolation of peptides and ¹⁸F-labeled peptides were performed using a Vydac protein and peptide column (218TP510; 5 μm, 250×10 mm). The flow rate was set at 5 mL/min, with the mobile phase starting from 95% solvent A (0.1% trifluoroacetic acid [TFA] in water) and 5% solvent B (0.1% TFA in acetonitrile [ACN]) (0-2 min) to 35% solvent A and 65% solvent B at 32 min. The analytical HPLC was performed using the same gradient system, but with a Vydac column (218TP54, 5 μm, 250×4.6 mm) and a flow rate of 1 mL/min. The Ultraviolet (UV) absorbance was monitored at 218 nm and the identification of the peptides was confirmed by separate standard injection.

### Preparation of Alkyne-Tosylate (Structure 1)

The alkyne-tosylate (structure 1) (FIG. 4-1) was prepared by using modified method reported by Burgess (Chem Commun (Camb), 1652-4, which is incorporated herein by reference for the corresponding discussion). In brief, sodium hydride (1 g, 25 mmol, 60%) was slowly added to the THF solution of triethylene glycol (5.8 g, 38 mmol) at 0° C. The mixture was stirred for 30 min and propargyl bromide (2.1 mL, 19 mmol) was then added dropwise. The mixture was stirred at room temperature for 18 h and the triethylene glycol alkyne was obtained as light yellow oil after purification by chromatography (2.5 g, 70%). ¹H NMR (400 MHz, CDCl₃) δ 4.13 (d, J=2.5 Hz, 2H), 3.61-58 (m, 10H), 3.52-3.50 (m, 2H), 2.75 (br, 1H), 2.38 (t, J=2.5 Hz, 1H). After the triethylene glycol alkyne (1 g, 5.4 mmol) was reconstituted in ACN (15 mL) and triethylamine (2 mL, 14 mmol), p-toluenesulfonyl chloride (2.1 g, 11 mmol) was added slowly and the mixture was stirred at room temperature for 16 h. After the reaction was quenched followed by general workup, the crude product was purified by flash chromatography to afford the alkyne-tosylate (structure 1) (1.5 g, 81%) as a colorless oil. ¹H NMR (400 MHz, CDCl₃) δ 7.75 (d, J=8.4 Hz, 2 H), 7.30 (d, J=8.4 Hz, 2 H), 4.14-4.06 (m, 4H), 3.65-3.58 (m, 6H), 3.55-3.52 (m, 4H), 2.38 (s, 3H), 2.37 (t, J=2.5 Hz, 1H).

### Preparation of Azido-RGD2

The 5-azidopentanoic acid was obtained as colorless oil according to the procedure published by Carrie (36). ¹H NMR (400 MHz, CDCl₃) δ 3.25 (t, J=6.5 Hz, 2 H), 2.34 (t, J=7.1 Hz, 2 H), 1.68-1.59 (m, 4H). The azido-RGD2 was prepared from cyclic RGD dimer E[c(RGDyK)]₂ (denoted as RGD2). To a solution of 5-azidopentanoic acid (18.6 mg, 0.13 mmol) and 20 μL DIPEA in ACN (0.5 mL), O-(N-Succinimidyl)-1,1,3,3-tetramethyl-uronium tetrafluoroborate (TSTU, 27 mg, 0.09 mmol) was added. The reaction mixture was stirred at room temperature for 0.5 h and then added to E[c(RGDyK)]₂ (20 mg, 14.8 μmol) in N,N′-dimethylformamide (DMF). The reaction was stirred at room temperature for another 2 h and the desired product azido-RGD2 was isolated by preparative HPLC. The collected fractions were combined and lyophilized to give a white fluffy powder (12 mg, 57% yield) with a retention time of 14.8 min on analytical HPLC. MALDI-TOF-MS: m/z 1475.87 for [MH]⁺ (C₆₄H₉₅N₂₂O₁₉, calculated molecular weight [MW] 1475.71).

### Preparation of Fluoro-PEG-Triazole-E(RGDyK)₂ (FPTA-RGD2)

To a solution of alkyne-tosylate (structure 1) (6.8 mg, 0.02 mmol) in ACN, powdered potassium fluoride (6 mg, 0.10 mmol), potassium carbonate (3 mg) and Kryptofix 222 (15 mg) were added and the mixture was heated at 90° C. for 40 min. The reaction mixture was evaporated to dryness and the residue was redissolved in 0.4 mL water and 0.4 mL THF. Azido-RGD2 (1 mg, 0.7 μmol) was then added followed by CuSO₄ (100 μL, 0.1 N) and sodium L-ascorbate (100 μL, 0.3 N) solution. The resulting mixture was stirred at room temperature for 24 h and the reaction was then quenched and purified by semi-preparative HPLC. The final product fluoro-PEG-triazole-E(RGDyK)₂ (FPTA-RGD2) was obtained in 81% yield (0.91 mg) with a retention time of 13.4 min on analytical HPLC. MALDI-TOF-MS: m/z 1665.82 for [MH]⁺ (C₇₃H₁₁₀FN₂₂O₂₂, calculated [MW] 1665.81).

### Radiochemistry

[¹⁸F]Fluoride was prepared by the ¹⁸O(p,n)¹⁸F nuclear reaction and it was then adsorbed onto an anion exchange resin cartridge. Kryptofix 222/K₂CO₃ solution (1 mL 9:1 ACN/water, 15 mg Kryptofix 222, 3 mg K₂CO₃) was used to elute the cartridge and the resulting mixture was dried in a glass reactor. A solution of alkyne-tosylate (structure 1) (4 mg in 1 mL ACN/DMSO) was then added and the resulting mixture was heated at the desired temperature (Table 1, Example 3). After cooling, the reaction was quenched and the mixture was injected onto a semi-preparative HPLC for purification. The collected radioactive peak was diluted in water (10 mL) and passed through a C18 cartridge. The trapped activity was then eluted off the cartridge with 1 mL THF and used for the next reaction. To the reactor vial with azido-RGD2 (1 mg), 37 MBq activity, CuSO₄ (100 μL, 0.1N) and sodium L-ascorbate (100 μL, 0.3 N) were added sequentially. The resulting mixture was heated at 40° C. for 20 min and the reaction was then quenched and purified by semi-preparative HPLC. The final product ¹⁸F-FPTA-RGD2 (Rt: 13.4 min, decay corrected yield 69±11%, radiochemical purity>97%) was concentrated and formulated in saline (0.9%, 500 μL) for in vivo studies.

### Octanol-Water Partition Coefficient

Approximately 111 kBq of ¹⁸F-FPTA-RGD2 in 500 μL of PBS (pH 7.4) were added to 500 μL of octanol in an Eppendorf microcentrifuge tube (model 5415R, Brinkman). The mixture was vigorously vortexed for 1 min at room temperature and centrifuged at 12,500 rpm for 5 min. After centrifugation, 200 μL aliquots of both layers were measured using a γ-counter (Packard Instruments). The experiment was carried out in triplicates.

### Cell Line and Animal Models

U87MG human glioblastoma cells were grown in Dulbecco's medium (Gibco) supplemented with 10% fetal bovine serum (FBS), 100 IU/mL penicillin, and 100 μg/mL streptomycin (Invitrogen Co.). Animal procedures were performed according to a protocol approved by Stanford University Institutional Animal Care and Use Committee. U87MG xenograft model was generated by subcutaneous (s.c.) injection of 1×10⁷ U87MG cells (integrin αᵥβ₃-positive) into the front flank of female athymic nude mice. Three to four weeks after inoculation (tumor volume: 100-400 mm³), the mice (about 9-10 weeks old with 20-25 g body weight) were used for microPET studies.

### Cell Integrin Receptor-Binding Assay

In vitro integrin-binding affinity and specificity of E[c(RGDyK)]₂ and FPTA-RGD2 were assessed via competitive cell binding assays using ¹²⁵I-echistatin as the integrin αᵥβ₃-specific radioligand (J Nucl Med 46, 1707-18, which is incorporated herein by reference for the corresponding discussion). The best-fit 50% inhibitory concentration (IC₅₀) values for U87MG cells were calculated by fitting the data with nonlinear regression using GraphPad Prism (GraphPad Software, Inc.). Experiments were performed with triplicate samples.

### In Vivo Metabolic Stability Studies

The metabolic stability of ¹⁸F-FPTA-RGD2 was evaluated in an athymic nude mouse bearing a U87MG tumor. Sixty min after intravenous injection of 2 MBq of ¹⁸F-FPTA-RGD2, the mouse was sacrificed and relevant organs were harvested. The blood was collected and immediately centrifuged for 5 min at 13,200 rpm. Liver, kidneys and tumor were homogenized and then centrifuged for 5 min at 13,200 rpm. After removal of the supernatants, the pellets were washed with 1 mL PBS. For each sample, supernatants of both centrifugation steps of blood, liver, and kidneys were combined and passed through C18 Sep-Pak cartridges. The urine sample was directly diluted with 1 mL of PBS and passed through a C18 Sep-Pak cartridge. The cartridges were each washed with 2 mL of water and eluted with 2 mL of ACN containing 0.1% TFA. After evaporation of the solvent, the residues were redissolved in 1 mL PBS and were injected onto the analytical HPLC. The eluent was collected with a fraction collector (0.5 min/fraction) and the radioactivity of each fraction was measured with the γ-counter.

microPET Studies

PET scans and image analysis were performed using a microPET R4 rodent model scanner (Siemens Medical Solutions) as previously reported (J Nucl Med 46, 1707-18 and J Nucl Med 47, 113-21, which is incorporated herein by reference for the corresponding discussion). About 2 MBq of ¹⁸F-FPTA-RGD2 was intravenously injected into each mouse (n=3) under isoflurane anesthesia (1-3%) and then subjected to a 30-min dynamic scan (1×1 min, 1×1.5 min, 1×3.5 min, 3×5 min, 1×6 min, total of 7 frames) starting from 1 min p.i. Five min static PET images were also acquired at 1 and 2 h p.i. For each microPET scan, regions of interest (ROIs) were drawn over the tumor, normal tissue, and major organs on decay-corrected whole-body coronal images. The radioactivity concentration (accumulation) within a tumor was obtained from the mean value within the multiple ROIs and then converted to % ID/g (J Nucl Med 46, 1707-18, which is incorporated herein by reference for the corresponding discussion). For a receptor-blocking experiment, mice bearing U87MG tumors on the front left flank were scanned (5 min static) after co-injection with ¹⁸F-FPTA-RGD2 (2 MBq) and c(RGDyK) (10 mg/kg).

### Statistical Analysis

Quantitative data were expressed as mean±SD. Means were compared using One-way ANOVA and student's t-test. P values<0.05 were considered statistically significant.

### Results:

### Chemistry and Radiochemistry

Both alkyne-tosylate (structure 1) and azido-RGD2 were obtained in high yields (FIG. 4-1). The alkyne-fluoride was prepared in situ and could be used directly for the reaction with azido-RGD2 to make the cold standard, which was purified by HPLC and confirmed by MALDI-TOF mass spectrometry. ¹⁸F-alkyne was also obtained in high yield at various conditions (Table 1, Example 3). The presence of acetonitrile may lower the labeling yield to some extent (Table 1, entry 1-3, Example 3). Although entry 5 gave the highest decay corrected yield (84.3±2.1%), the non decay corrected yield was 69.8%, which is actually slightly lower than the non decay corrected yield from entry 4 (71.4%). Thus, the condition from entry 4 was used for the subsequent studies. We also noted that the ¹⁸F-alkyne intermediate had to be purified before the conjugation with azido-RGD2 to guarantee high labeling yield (This might due to the removal of large excess amount of unreacted alkyne). The radiochemical purity of the ¹⁸F-labeled peptide ¹⁸F-FPTA-RGD2 was higher than 97% according to analytical HPLC. The specific radioactivity of ¹⁸F-FPTA-RGD2 was determined to be about 100-200 TBq/mmol based on the labeling agent ¹⁸F-SFB, as the unlabeled azido-RGD2 was efficiently separated from the product.

The octanol/water partition coefficient (logP) for ¹⁸F-FPTA-RGD2 was −2.71±0.006, indicating that the tracer is slightly more hydrophilic than ¹⁸F-FB-RGD2 (¹⁸F-FRGD2, −2.103±0.030) and ¹⁸F-FB-PEG3-RGD2 (¹⁸F-FPRGD2, −2.280±0.054) (18F-labeled mini-PEG spacered RGD dimer (¹⁸F-FPRGD2): synthesis and microPET imaging of αᵥβ₃integrin expression. Eur J Nucl Med Mol Imaging., which is incorporated herein by reference for the corresponding discussion).

### In Vitro Cell Integrin Receptor-Binding Assay

The receptor-binding affinity of RGD2 and FPTA-RGD2 was determined by performing competitive displacement studies with ¹²⁵I-echistatin. All peptides inhibited the binding of ¹²⁵I-echistatin (integrin αᵥβ₃ specific) to U87MG cells in a concentration dependent manner. The IC₅₀ values for RGD2 and FPTA-RGD2 were 79.2±4.2 and 144±6.5 nM, respectively (n=3) (FIG. 4-2). In a parallel experiment, the IC₅₀ value for FPRGD2 was 97±4.8 nM. The comparable IC₅₀ values of these compounds suggest that the introduction of miniPEG linker and triazole group had little effect on the receptor binding affinity.

microPET Imaging of U87MG Tumor-Bearing Mice

Dynamic microPET scans were performed on U87MG xenograft model and selected coronal images at different time points after injecting ¹⁸F-FPTA-RGD2 were shown in FIG. 4-3A. Good tumor-to-contralateral background contrast was observed as early as 10 min after injection (5.4±0.7% ID/g). The U87MG tumor uptake was 3.1±0.6, 2.1±0.4, and 1.3±0.4% ID/g at 0.5, 1, and 2 h p.i., respectively (n=3). Most activity in the non-targeted tissues and organs were cleared by 1 h p.i. For example, the uptake values in the kidney, liver, and muscle were as low as 2.7±0.8, 1.9±0.4, and 1.0±0.3% ID/g, respectively at 1 h p.i. The averaged time-activity curves (TACs) for the U87MG tumor, liver, kidney and muscle were shown in FIG. 4-4. ¹⁸F-FPTA-RGD2 was cleared mainly through the kidneys. Some hepatic clearance was also observed. The integrin αᵥβ₃ specificity of ¹⁸F-FPTA-RGD2 in vivo was confirmed by a blocking experiment where the tracer was co-injected with c(RGDyK) (10 mg/kg). As can be seen from FIG. 4-3B, the U87MG tumor uptake in the presence of non-radiolabeled RGD peptide (0.9±0.3% ID/g) is significantly lower than that without RGD blocking (2.1±0.4% ID/g) (P<0.05) at 1 h p.i.

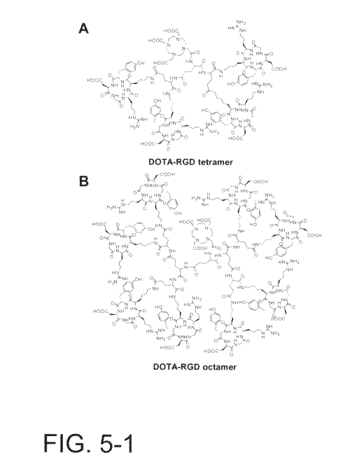

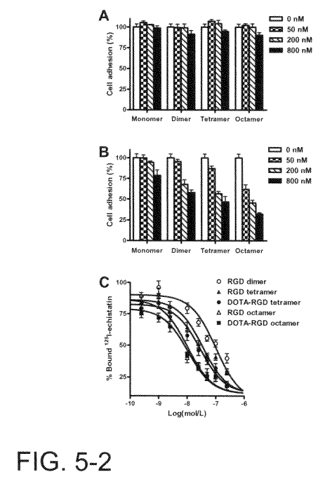

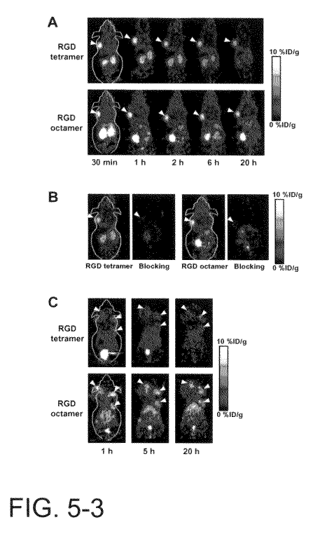

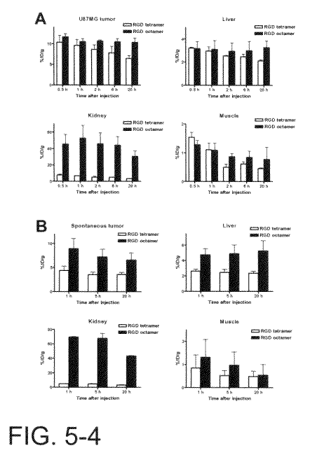

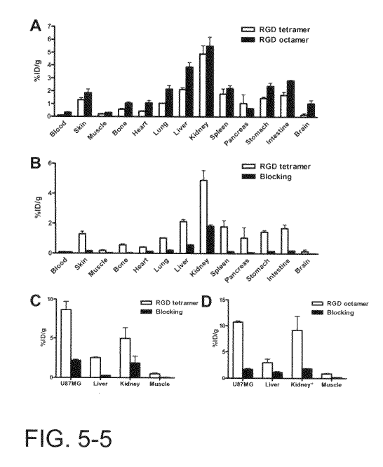

The comparison of tumor and various organ uptake of ¹⁸F-FPTA-RGD2 with ¹⁸F-FPRGD2 and ¹⁸F-FRGD2 were shown in FIG. 4-5. The uptake in the U87MG tumor was slightly lower for ¹⁸F-FPTA-RGD2 which might be caused by integrin αᵥβ₃ binding affinity difference (FIG. 4-5A). The kidney uptake for these three tracers was comparable (FIG. 4-5B) and the clearance rate was highest for ¹⁸F-FPTA-RGD2. ¹⁸F-FPTA-RGD2 had lowest liver uptake which was consistent with the hydrophilic sequence of these three compounds (FIG. 4-5C). The non-specific uptake in the muscle was at a very low level for all three compounds (FIG. 4-5D).

### In Vivo Metabolic Stability Studies

The metabolic stability of ¹⁸F-FPTA-RGD2 was determined in mouse blood and urine and the in liver, kidney and tumor homogenates at 1 h after intravenous injection of radiotracer into a U87MG tumor-bearing mouse. The extraction efficiency of all organs was between 86% and 99% (Table 2, Example 3). The lowest extraction efficiency was found for the kidney. There are 1% to 41% of the total activity could not be trapped on the C-18 cartridges, which can be related to very hydrophilic metabolites and protein-bound activity. After ACN elution, the radioactivity of each sample was injected onto an analytical HPLC and the HPLC chromatograms are shown in FIG. 4-6. The fraction of intact tracer was between 75% and 99% (Table 2, Example 3). Although we did not identify the metabolites, we found that all metabolites eluted earlier from the HPLC column than the parent compound (FIG. 4-6), which behaved similarly to ¹⁸F-FRGD2 (J Nucl Med 47, 113-21, which is incorporated herein by reference for the corresponding discussion) and ¹⁸F-FPRGD2 (18F-labeled mini-PEG spacered RGD dimer (¹⁸F-FPRGD2): synthesis and microPET imaging of αᵥβ₃ integrin expression., Eur J Nucl Med Mol Imaging, which is incorporated herein by reference for the corresponding discussion).

### Discussion

¹⁸F-labeling of cyclic RGD peptide was first reported by Haubner et al. (Bioconjug Chem 15, 61-9, which is incorporated herein by reference for the corresponding discussion). A monomeric glycopeptide based on c(RGDfK) was ¹⁸F-radiolabeled via ¹⁸F-2-fluoropropionate prosthetic group and the resulting ¹⁸F-galacto-RGD exhibited integrin αᵥβ₃ specific tumor uptake in integrin-positive xenograft models. Initial clinical trials in a limited number of cancer patients revealed that this tracer can be safely given to patients and is able to delineate certain lesions that are integrin positive (Clin Cancer Res 12, 3942-9, which is incorporated herein by reference for the corresponding discussion). We have ¹⁸F-radiolabeled both mono and dimeric RGD peptides using an ¹⁸F-4-fluorobenzoyl (¹⁸F-FB) prosthetic group (Mol Imaging Biol 8, 9-15 and J Nucl Med 47, 113-21, each of which is incorporated herein by reference for the corresponding discussion). The dimeric RGD peptide tracer, ¹⁸F-FB-E[c(RGDyK)]₂ (denoted as ¹⁸F-FRGD2), exhibited excellent integrin αᵥβ₃-specific tumor imaging with favorable in vivo pharmacokinetics (J Nucl Med 47, 113-21 and Mol Imaging 3, 96-104, each of which is incorporated herein by reference for the corresponding discussion). The binding potential extrapolated from Logan plot graphical analysis of the PET data correlated well with the receptor density measured by SDS-PAGE/autoradiography in various xenograft models. The tumor-to-background ratio at 1 h after injection of ¹⁸F-FRGD2 also gave a good linear relationship with the tumor tissue integrin αᵥβ₃ expression level (J Nucl Med 47, 113-21, which is incorporated herein by reference for the corresponding discussion). We have also reported a thiol-reactive synthon, N-[2-(4-¹⁸F-fluorobenzamido)ethyl]maleimide (¹⁸F-FBEM), for labeling mono and dimeric sulfhydryl-RGD peptides (J Nucl Med 47, 1172-80, which is incorporated herein by reference for the corresponding discussion). To extend our efforts of ¹⁸F-radiolabeling strategies, we explored and reported the possibility to label dimeric RGD peptide E[c(RGDyK)]₂ using Hsuigen 1,3-dipolar cycloaddition reaction (one of the “click chemistry” reactions) and evaluated the ability of the new PET tracer for integrin αᵥβ₃ targeting in vitro and in vivo.

Alkyne-tosylate (structure 1) was designed as the labeling precursor which allowed nucleophilic fluorination and displacement of the tosyl group to occur in high yield under mild conditions (15 min, 78.5±2.3% yield). A triethylene glycol liker was employed in the structure to reduce volatility and obtain water solubility. The azido group was introduced to RGD dimer RGD2 by reacting the glutamate amine group with the azido-NHS ester. A robust catalytic system, Cu²⁺/ascorbate, was used for the labeling reaction (Angew Chem Int Ed Engl 41, 2596-9, which is incorporated herein by reference for the corresponding discussion). In comparison with the SFB labeling procedure (starting from ¹⁸F-F⁻, the total synthesis time of ¹⁸F-FPRGD2 was about 180 min with an overall non-decay-corrected yield of 12.9% (decay-corrected yield 40%)) (18), click labeled ¹⁸F-FPTA-RGD2 could be obtained in 110 min with 26.9% non-decay-corrected yield (decay-corrected yield 53.8%). The reduced reaction time and increased labeling yield make ‘click chemistry’ a valuable method for labeling RGD peptide with ¹⁸F.

We also studied the application of ¹⁸F-FPTA-RGD2 for in vivo imaging. We found that this tracer had good tumor-to-muscle ratio and predominant renal excretion. Compared with ¹⁸F-FPRGD2 and ¹⁸F-FRGD2, the tumor targeting efficacy of ¹⁸F-FPTA-RGD2 was decreased to some extent which might be caused by the slightly decreased integrin binding affinity based on cell binding assay. The unspecific blood pool activity could be another factor. However, no significant difference was observed for these compounds (P>0.5) (FIG. 4-5E). ¹⁸F-FPTA-RGD2 also had faster clearance rate and lower liver uptake which might due to the increased hydrophilicity of this tracer (logP=−2.710±0.006), after the replacement of benzoic group with a short PEG linker. Metabolic stability study revealed that the triazoles unit, formed by click chemistry in ¹⁸F-FPTA-RGD2, has comparable in vivo stability compared with the amide bound made from SFB in the case of ¹⁸F-FRGD2 and ¹⁸F-FPRGD2 (Eur J Nucl Med Mol Imaging (see above) and J Nucl Med 47, 113-21, each of which is incorporated herein by reference for the corresponding discussion).

This Example demonstrated that RGD peptide can be labeled efficiently through the ‘Click Chemistry’. The major advantage of ¹⁸F-FPTA-RGD2 would be shortened reaction time, increased labeling yield, and comparable in vivo stability. The tumor targeting efficacy of this tracer was comparable with SFB-labeled RGD peptides and can be further improved. First, the relatively long linker (triethylene glycol plus four methylene group) in ¹⁸F-FPTA-RGD2 might account for the decreased intergin binding affinity. Our future work will focus on the development of various linkers suitable for this new labeling method and study the in vivo pharmacokinetics of the resulting tracers. Second, high αᵥβ₃ binding affinity is needed to afford high tumor uptake and retention. Based on polyvalency effect, tetrameric RGD peptide (J Nucl Med 46, 1707-18, which is incorporated herein by reference for the corresponding discussion), labeled with the synthon described here, would have more effective binding to integrin αᵥβ₃ and better tumor targeting efficacy. Third, the click labeling method developed here could also be applied to label a variety of other peptides, proteins, antibodies or oligonucleotides after the introduction of the azido group. Due to the mild labeling conditions, ¹⁸F might be easily engineered to incorporate the organo azide residue without compromising the biological activity.

### Conclusions

The new tracer ¹⁸F-FPTA-RGD2 was synthesized with high specific activity based on ‘click chemistry’. This tracer exhibited good tumor-targeting efficacy, relatively good metabolic stability, as well as favorable in vivo pharmacokinetics. The new ¹⁸F labeling method developed in this study, could also have a general application in labeling azido-containing bioactive molecules in high radiochemical yield and high specific activity for successful PET applications.

TABLE 1
Example 3. Radiolabeling yields (decay-corrected) of ¹⁸F-fluoro-PEG-
alkyne intermediate at various conditions (n = 3).

| Entry | Solvent | Temperature & time | Yield (%) |
|---|---|---|---|
| 1 | ACN | 90° C. for 15 min | 61.2 ± 2.5 |
| 2 | ACN | 110° C. for 15 min | 71.4 ± 3.0 |
| 3 | ACN/DMSO | 110° C. for 15 min | 75.0 ± 1.8 |
| 4 | DMSO | 110° C. for 15 min | 78.5 ± 2.3 |
| 5 | DMSO | 110° C. for 30 min | 84.3 ± 2.1 |

TABLE 2
Example 3. Extraction efficiency, elution efficiency, and HPLC analysis of
soluble fraction of tissue homogenates at 1 h post-injection of
¹⁸F-FPTA-RGD2 (“ND” denotes “not determined”).

| | | | | | |
|---|---|---|---|---|---|
| Fraction | Blood | Urine | Liver | Kidney | U87MG |

Extraction efficiency (%)

| | | | | | |
|---|---|---|---|---|---|
| Insoluble fraction | 0.8 | ND | 10.3 | 13.3 | 7.5 |
| Soluble fraction | 99.2 | ND | 89.7 | 86.7 | 92.5 |

Elution efficiency (%)

| | | | | | |
|---|---|---|---|---|---|
| Unretained fraction | 2.8 | 0.4 | 33.9 | 12.8 | 18.5 |
| Wash water | 8.8 | 0.5 | 7.4 | 3.9 | 5.2 |
| Acetonitrile eluent | 88.4 | 99.1 | 58.7 | 83.3 | 76.4 |

HPLC analysis (%)

| | | | | | |
|---|---|---|---|---|---|
| Intact tracer | 75.9 | 99.7 | 81.6 | 89.1 | 82.4 |

### Example 4

### Introduction

Integrin αᵥβ₃ plays a critical role in tumor angiogenesis and metastasis. Suitably radiolabeled cyclic RGD peptides can be used for noninvasive imaging of αᵥβ₃ expression and targeted radionuclide therapy. In this Example we developed ⁶⁴Cu-labeled multimeric RGD peptides, E{E[c(RGDyK)]₂}₂ (RGD tetramer) and E(E{E[c(RGDyK)]₂}₂)₂ (RGD octamer), for positron emission tomography (PET) imaging of tumor integrin αᵥβ₃ expression. In particular, the Example describes the design, synthesis, and evaluation of the new tetrameric and octameric RGD peptides based on the polyvalency principle. These multimeric RGD peptides were constructed on the c(RGDyK) motif with glutamate as the branching unit. They were conjugated with the macrocylic chelator 1,4,7,10-tetraazacyclododecane-N,N′,N″,N′″-tetraacetic acid (DOTA) and labeled with ⁶⁴Cu for microPET imaging of integrin αᵥβ₃ expression in both the c-neu oncomouse model (murine mammary carcinoma) and a subcutaneous U87MG xenograft (human glioblastoma) model.

Both RGD tetramer and RGD octamer were synthesized with glutamate as the linker. After conjugation with 1,4,7,10-tetra-azacyclododecane-N,N′,N″,N′″-tetraacetic acid (DOTA), the peptides were labeled with ⁶⁴Cu for biodistribution and microPET imaging studies (U87MG human glioblastoma xenograft model and c-neu oncomouse model). Cell adhesion assay, cell binding assay, receptor blocking experiments, and immunohistochemistry were also carried out to evaluate the αᵥβ₃ binding affinity/specificity of the RGD peptide-based conjugates in vitro and in vivo.

The RGD octamer had significantly higher αᵥβ₃ integrin binding affinity and specificity than the RGD tetramer analog (IC₅₀ value was 10 nM for octamer versus 35 nM for tetramer). ⁶⁴Cu-DOTA-RGD octamer had higher tumor uptake and longer tumor retention than ⁶⁴Cu-DOTA-RGD tetramer in both tumor models tested. Integrin αᵥβ₃ specificity of both tracers was confirmed by successful receptor blocking experiments. The high uptake and slow clearance of ⁶⁴Cu-DOTA-RGD octamer in the kidneys is mainly attributed to the integrin positiveness of the kidneys, significantly higher integrin αᵥβ₃ binding affinity, and larger molecular size of the octamer as compared to the other RGD analogs. Polyvalency has a profound effect on the receptor binding affinity and in vivo kinetics of radiolabed RGD multimers.

### Materials and Methods

All commercially available reagents were used without further purification. DOTA was purchased from Macrocyclics, Inc. Dicycicohexylcarbodiimide (DCC), 1-ethyl-3-[3-(dimethylamino)propyl]carbodiimide (EDC), N-hydroxysulfonosuccinimide (SNHS), trifluoroacetic acid (TFA), and Chelex 100 resin (50-100 mesh) were purchased from Aldrich. Water and all buffers were passed through a Chelex 100 column (1×15 cm) before radiolabeling. Reversed-phase extraction C-18 Sep-Pak cartridges were obtained from Waters. The syringe filter and polyethersulfone membranes (pore size, 0.2 μm; diameter, 13 mm) were obtained from Nalge Nunc International. ¹²⁵I-echistatin (specific activity: 74,000 GBq/mmol) was purchased from GE Healthcare. Female athymic nude mice (4-6 weeks old) were supplied from Harlan. ⁶⁴Cu (half-life: 12.7 h; β⁺: 17.4%; β⁻: 30%) was obtained by utilizing the ⁶⁴Ni(p,n)⁶⁴Cu nuclear reaction from University of Wisconsin-Madison. The dimeric RGD peptide E[c(RGDyK)]₂ was synthesized by Peptides International, Inc. Analytical and semi-preparative reversed-phase high-performance liquid chromatography (RP-HPLC) were performed on a Dionex 680 chromatography system with a UVD 170U absorbance detector and model 105S single-channel radiation detector (Carroll & Ramsey Associates). Isolation of DOTA-conjugated peptides and ⁶⁴Cu-labeled peptides was performed using a Vydac protein and peptide column (218TP510; 5 μm, 250×10 mm). The flow rate was 3 mL/min for semi-preparative HPLC, with the mobile phase starting from 95% solvent A (0.1% TFA in water) and 5% solvent B (0.1% TFA in acetonitrile) (0-2 min) to 35% solvent A and 65% solvent B at 32 min. The analytical HPLC was performed with the same gradient system, but with a Vydac 218TP54 column (5 μm, 250×4.6 mm) at a flow rate of 1 mL/min. The UV absorbance was monitored at 218 nm.

Preparation of E{E[c(RGDyK)]₂}₂ (RGD Tetramer) and E(E{E[c(RGDyK)]₂}₂)₂ (RGD Octamer)

The Boc-protected glutamic acid activated ester Boc-E(OSu)₂ was prepared as previously reported (J Nucl Med. 2005;46:1707-1718, which is incorporated herein by reference for the corresponding discussion). To a solution of Boc-E(OSu)₂ (4.4 mg, 0.01 mmol) in 1 mL anhydrous N,N-dimethylformamide (DMF), three equivalence of RGD dimer (E[c(RGDyK)]₂, 40 mg, 0.03 mmol) or RGD tetramer was added. The pH of the resulting mixture was adjusted to 8.5-9.0 with diisopropylethyl amine (DIPEA). After stirring at room temperature for overnight, the desired product Boc-RGD tetramer or Boc-RGD octamer were isolated by preparative HPLC. The Boc-group was then removed by anhydrous TFA and the crude product was again purified by HPLC. 17 mg RGD tetramer was obtained as white powder with 58% overall yield (analytical HPLC retention time Rₜ: 13.3 min). MALDI-TOF-MS: m/z 2811.0 for [MH]⁺ (C₁₂₃H₁₈₀N₃₉O₃₈, calculated molecular weight [MW] 2811.3). RGD octamer was obtained in 46% overall yield (analytical HPLC Rₜ: 14.3 min). MALDI-TOF-MS: m/z 5735.5 for [MH]⁺ (C₂₅₁H₃₆₄N₇₉O₇₈, calculated MW 5734.7).

### DOTA Conjugation and Radiolabeling

DOTA was activated and conjugated to RGD multimers as reported earlier (J Nucl Med. 2005;46:1707-1718, which is incorporated herein by reference for the corresponding discussion). The DOTA-RGD multimers were purified by semi-preparative HPLC. Detailed ⁶⁴Cu-labeling procedure has been reported earlier (J Nucl Med. 2005;46:1707-1718, which is incorporated herein by reference for the corresponding discussion). In brief, 20 μL of ⁶⁴CuCl₂ (74 MBq in 0.1 N HCl) was diluted in 400 μL of 0.1 mol/L sodium acetate buffer (pH 6.5) and added to the DOTA-RGD multimer (1 mg/mL peptide solution was made and aliquoted. 5 μg of DOTA-RGD tetramer and 10 μg of DOTA-RGD octamer per 37 MBq of ⁶⁴Cu were used for the labeling respectively). The reaction mixture was incubated for 1 h at 50° C. ⁶⁴Cu-DOTA-RGD tetramer/octamer was then purified by semi-preparative HPLC and the radioactive peak containing the desired product was collected. After removal of the solvent by rotary evaporation, the residue was reconstituted in 800 μL phosphate-buffered saline (PBS) and passed through a 0.22-μm syringe filter for in vivo animal experiments.

### Cell Adhesion Assay

Ninety-six well plates were coated with 2 μg/mL of fibronectin or vitronectin (Sigma-Aldrich) in PBS at 4° C. overnight and treated with 2% bovine serum albumin (BSA) for 1 h at 37° C. U87MG cells (human glioblastoma, ATCC; 2×10⁵ cells/mL) with various concentrations of RGD multimers (50 nM, 200 nM, 800 nM) in 100 μL serum-free Dulbecco's modified Eagle's medium (DMEM) containing 0.1% BSA were incubated for 20 min at 37° C. The resulting mixture was added to the plates and incubated for 1 h at 37° C. Plates treated with BSA only were used as negative control. After removal of the medium by aspiration, 0.04% crystal violet solution was added and incubated for 10 min at room temperature. The wells were washed three times with PBS and 20 μL Triton X-100 were added for permeabilization. Distilled water (80 μL) was then added and the number of adherent cells was assessed with a microplate reader (Tecan; measurement wavelength: 550 nm; reference wavelength: 630 nm).

### Cell Integrin Receptor-Binding Assay

In vitro integrin-binding affinity and specificity of RGD multimers and DOTA-RGD multimers were assessed via competitive cell binding assays using ¹²⁵I-echistatin as the integrin αᵥβ₃-specific radioligand (J Nucl Med. 2005;46:1707-1718, which is incorporated herein by reference for the corresponding discussion). The best-fit 50% inhibitory concentration (IC₅₀) values for U87MG cells were calculated by fitting the data with nonlinear regression using Graph-Pad Prism with 450-600 KeV energy window (GraphPad Software, Inc.). Experiments were performed with triplicate samples.

### Animal Models

Animal procedures were performed according to a protocol approved by Stanford University Institutional Animal Care and Use Committee. U87MG xenograft model was generated by subcutaneous (s.c.) injection of 1×10⁷ U87MG cells (integrin αᵥβ₃-positive) into the front left flank of female athymic nude mice. Three to four weeks after inoculation (tumor volume: 100-400 mm³), the mice (about 9-10 weeks old with 20-25 g body weight) were used for biodistribution and microPET studies. The c-neu oncomouse (integrin αᵥβ₃-positive, Charles River Laboratories, Charles River, Canada) is a spontaneous tumor-bearing model that carries an activated c-neu oncogene driven by a mouse mammary tumor virus (MMTV) promoter. Transgenic mice uniformly expressing the MMTV/c-neu gene develop mammary adenocarcinomas (4 to 8 months postpartum) that involve the entire epithelium in each gland. The animals were scanned at 7 months old at about 20 g body weight and the tumors were on both sides of the body.

### Biodistribution Studies

Female nude mice were injected with 0.74-1.11 MBq of ⁶⁴Cu-DOTA-RGD tetramer or ⁶⁴Cu-DOTA-RGD octamer to evaluate the distribution of these tracers in the major organs of mice (J Nucl Med. 2005;46:1707-1718, which is incorporated herein by reference for the corresponding discussion). Blocking experiment was also performed by co-injecting radiotracer with a saturating dose of c(RGDyK) (10 mg/kg of mouse body weight). All mice were sacrificed and dissected at 20 h post-injection (p.i.) of the tracer. Blood, U87MG tumor, major organs and tissues were collected and wet weighed. The radioactivity in the tissue was measured using a γ-counter (Packard). The results were presented as percentage injected dose per gram of tissue (% ID/g). For each mouse, the radioactivity of the tissue samples was calibrated against a known aliquot of the injectate and normalized to a body mass of 20 g. Values were expressed as mean±SD for a group of 3 animals.

microPET Studies

PET scans and image analysis were performed using a microPET R4 rodent model scanner (Siemens Medical Solutions) as previously reported (J Nucl Med. 2005;46:1707-1718 and J Nucl Med. 2006;47:113-121, each of which is incorporated herein by reference for the corresponding discussion). About 9.3 MBq of ⁶⁴Cu-DOTA-RGD multimer was intravenously injected into each mouse under isoflurane anesthesia. Five minute static scans were acquired at 30 min, 1 h, 2 h, 6 h, and 20 h p.i. The images were reconstructed by a 2-dimensional ordered-subsets expectation maximum (OSEM) algorithm and no correction was applied for attenuation and scatter. For each microPET scan, regions of interest (ROIs) were drawn over the tumor, normal tissue, and major organs on decay-corrected whole-body coronal images. The radioactivity concentration (accumulation) within a tumor was obtained from the maximum value within the multiple ROIs and then converted to % ID/g. For a receptor-blocking experiment, mice bearing U87MG tumors on the front left flank were scanned (5-min static) after co-injection of 9.3 MBq of ⁶⁴Cu-DOTA-RGD multimer and 10 mg/kg c(RGDyK).

### Statistical Analysis

Quantitative data were expressed as mean±SD. Means were compared using One-way ANOVA and student's t-test. P values<0.05 were considered statistically significant.

### Results

### Chemistry and Radiochemistry

The synthesis of RGD tetramer and RGD octamer was performed through an active ester method by coupling Boc-E(OSu)₂ with RGD dime/tetramer followed by TFA deprotection. In aqueous solution, DOTA was activated with EDC/SNHS, and the resulting DOTA-OSSu was conjugated with RGD tetramer/octamer to yield DOTA-RGD tetramer and DOTA-RGD octamer (FIG. 5-1). DOTA-RGD tetramer was synthesized in 70% yield (analytical HPLC Rₜ: 14.5 min). MALDI-TOF-MS: m/z 3199.0 for [MH]⁺ (C₁₄₀H₂₀₇N₄₂O₄₅, calculated MW 3198.4). DOTA-RGD octamer was produced in 67% (analytical HPLC Rₜ: 14.5 min). MALDI-TOF-MS: m/z 6122.3 for [MH]⁺ (C₂₆₇H₃₉₀N₈₃O₈₅, calculated MW 6121.9). On the analytical HPLC, no significant difference in retention time was observed between ⁶⁴Cu-DOTA-RGD multimer and DOTA-RGD multimer. ⁶⁴Cu-labeling was achieved in 80-90% decay-corrected yield with radiochemical purity of >98%. The specific activity of ⁶⁴Cu-DOTA-RGD tetramer and ⁶⁴Cu-DOTA-RGD octamer was about 23 MBq/nmol (0.62 Ci/μmol).

Cell Adhesion assay

The effect of RGD multimers on U87MG cell adhesion ability was investigated. Both fibronectin and vitronectin are ligands for integrin αᵥβ₃. Fibronectin binds to several other integins besides αᵥβ₃ while vitronectin is integrin αᵥβ₃ specific (Annu Rev Cell Dev Biol. 1996;12:697-715 and Cancer Res. 2005;65:113-120, each of which is incorporated herein by reference for the corresponding discussion). For fibronectin coated plates, no significant difference in U87MG cell adhesion ability was observed in the presence of RGD multimers at the tested concentration range (FIG. 5-2A). For vitronectin coated plates, RGD multimers inhibited the cell adhesion in a concentration dependent manner. The ability of different RGD peptides to inhibit cell adhesion at the same concentration followed the order of monomer<dimer<tetramer<octamer (FIG. 5-2B). The calculated IC₅₀ values for RGD monomer, dimer, tetramer and octamer were (2.7±0.7)×10⁻⁶, (7.0±1.0)×10⁻⁷, (3.2±0.9)×10⁻⁷ and (1.1±0.2)×10⁻⁷ mol/L, respectively. RGD octamer was three times as effective as the RGD tetramer and 27 times as effective as the RGD monomer.

### Cell Binding Assay

We compared the receptor-binding affinity of RGD dimer, tetramer, octamer, DOTA-RGD tetramer, and DOTA-RGD octamer using competitive cell binding assay (FIG. 5-2C). All peptides inhibited the binding of ¹²⁵I-echistatin to αᵥβ₃ integrin-positive U87MG cells in a dose-dependent manner. The IC₅₀ values for RGD dimer, tetramer and octamer, were (1.0±0.1)×10⁻⁷, (3.5±0.3)×10⁻⁸, and (1.0±0.2)×10⁻⁸ mol/L, respectively (n=3). DOTA conjugation had minimal effect on the receptor binding avidity and the IC₅₀ values for DOTA-RGD tetramer and DOTA-RGD octamer were (2.8±0.4)×10⁻⁸ and (1.1±0.2)×10⁻⁸ mol/L, respectively. Cell binding assay demonstrated that RGD tetramer had about 3-fold higher integrin αᵥβ₃ avidity than the RGD dimer, and the RGD octamer further increased the integrin avidity by another 3-fold (attributed to the polyvalency effect). It is of note that the IC₅₀ values measured from such cell binding assay are always lower than those obtained from purified αᵥβ₃ integrin protein fixed on a solid matrix (e.g., ELISA and solid-phase receptor binding assay) (J Nucl Med. 2001;42:326-336, which is incorporated herein by reference for the corresponding discussion).

microPET Imaging of U87MG Tumor-Bearing Mice and c-Neu Oncomice

The tumor targeting efficacy of ⁶⁴Cu-DOTA-RGD tetramer and ⁶⁴Cu-DOTA-RGD octamer in U87MG tumor-bearing nude mice (n=3/tracer) were evaluated by multiple time-point static microPET scans. Representative decay-corrected coronal microPET images at different time points postinjection (p.i.) are shown in FIG. 5-3A. The U87MG tumors were clearly visualized with high tumor-to-background contrast for both tracers. The uptake of ⁶⁴Cu-DOTA-RGD tetramer in U87MG tumors was rapid and high, reaching 10.3±1.6, 9.6±1.4, 8.6±1.0, 7.7±1.6, 6.4±0.7% ID/g at 0.5, 1, 2, 6 and 20 h p.i., respectively (FIG. 5-4A). The activity accumulation of ⁶⁴Cu-DOTA-E{E[c(RGDyK)]₂}₂ (the D-Tyr analog) in U87MG tumor was slightly higher than ⁶⁴Cu-DOTA-E{E[c(RGDfK)]₂}₂ (the D-Phe analog) (J Nucl Med. 2005;46:1707-1718, which is incorporated herein by reference for the corresponding discussion) and no significant difference in the liver and kidney uptake was observed between the D-Tyr and D-Phe RGD tetramer analogs, similar as previously reported for the RGD dimers (Mol Imaging Biol. 2004;6:350-359, which is incorporated herein by reference for the corresponding discussion).

The uptake of ⁶⁴Cu-DOTA-RGD octamer in U87MG tumor was higher than ⁶⁴Cu-DOTA-RGD tetramer at all time points examined, reaching 11.7±0.7, 10.6±0.7, 10.6±0.3, 10.5±0.7, 10.3±1.0% ID/g at 0.5, 1, 2, 6 and 20 h p.i., respectively (FIG. 5-4A). There was minimal wash out from the tumor during the experimental time span (20 h). Activity accumulation in the liver, kidneys, and the muscle was also shown in FIG. 5-4A. The uptake of the two tracers in the liver and muscle was similar while the kidney uptake of ⁶⁴Cu-DOTA-RGD octamer was much higher than the ⁶⁴Cu-DOTA-RGD tetramer. Representative coronal images of U87MG tumor-bearing mice with and without coinjection of a blocking dose of c(RGDyK) (10 mg/kg) were illustrated in FIG. 5-3B. The tracer uptake in the U87MG tumor was significantly reduced in the presence of c(RGDyK) in both cases (2.2±0.1% ID/g vs. 8.6±1.0% ID/g for ⁶⁴Cu-DOTA-RGD tetramer and 1.7±0.2% ID/g vs. 10.6±0.3% ID/g for ⁶⁴Cu-DOTA-RGD octamer at 2 h p.i., respectively), indicating the in vivo integrin αᵥβ₃ binding specificity of both tracers. The uptake of both tracers in all the other organs was also significantly lower, similar as those observed for other RGD peptide-based tracers (J Nucl Med. 2006;47:1172-1180, which is incorporated herein by reference for the corresponding discussion).

The c-neu oncomouse model has been characterized with radiometal labeled RGD peptides other than ⁶⁴Cu. ¹¹¹In-DOTA-E[c(RGDfK)]₂ and ⁹⁰Y-DOTA-E[c(RGDfK)]₂ had ˜3.0% ID/g at 2 h and −1.5% ID/g at 24 h p.i. while their monomeric counterparts had only ˜1.3% ID/g at 2 h and −0.5% ID/g at 24 h p.i., respectively (Top Curr Chem. 2005;252:117-153, which is incorporated herein by reference for the corresponding discussion). The tumor uptake of our newly developed ⁶⁴Cu-DOTA-RGD tetramer and ⁶⁴Cu-DOTA-RGD octamer in this spontaneous mammary carcinoma model was studied. The decay-corrected coronal microPET images are shown in FIG. 5-3C and the quantitative data are shown in FIG. 5-4B. The tumor uptake of ⁶⁴Cu-DOTA-RGD tetramer reached 4.4±0.9% ID/g (n=3) at 1 h p.i. with slow clearance (3.6±0.4% ID/g at 20 h p.i.). For ⁶⁴Cu-DOTA-RGD octamer, the tumor uptake was 8.9±2.1% ID/g (n=3) at 1 h p.i., almost twice as high as the ⁶⁴Cu-DOTA-RGD tetramer. The tumor wash out was also slow, with the uptake being 6.6±1.5% ID/g at 20 h p.i.

The uptake in the liver of the oncomice was significantly higher for the ⁶⁴Cu-DOTA-RGD octamer than the ⁶⁴Cu-DOTA-RGD tetramer, which may be attributed to possible liver metastasis (FIG. 5-4B). All the mice have multiple tumors at 7 months old. Since the spontaneous tumor had much higher uptake of ⁶⁴Cu-DOTA-RGD octamer, the liver metastasis is expected to follow the same trend. The uptake in the muscle was similar for both tracers. The kidney uptake of ⁶⁴Cu-DOTA-RGD octamer in the c-neu oncomice is also much higher than ⁶⁴Cu-DOTA-RGD tetramer, similar to that observed in the athymic nude mice.

### Biodistribution Studies and Blocking Experiment

To investigate the localization of ⁶⁴Cu-DOTA-RGD tetramer and ⁶⁴Cu-DOTA-RGD octamer in normal athymic nude mice, biodistribution studies were carried out at 20 h p.i. As can be seen in FIG. 5-5A, the kidney uptake of ⁶⁴Cu-DOTA-RGD tetramer was 5.0±0.7% ID/g (n=3) while the uptake was almost 5-fold higher for the ⁶⁴Cu-DOTA-RGD octamer (27.0±3.5% ID/g, n=3). Due to the slower clearance, the uptake of ⁶⁴Cu-DOTA-RGD octamer was also slightly higher in most of the organs than the ⁶⁴Cu-DOTA-RGD tetramer. Biodistribution of ⁶⁴Cu-DOTA-RGD tetramer in female athymic nude mice with and without a blocking dose of c(RGDyK) are shown in FIG. 5-5B and significant decrease of radioactivity in the kidney and all other dissected tissues was observed. Quantitative data of the microPET scans shown in FIG. 5-3B are presented in FIG. 5-5C and 5-5D. Excess amount of c(RGDyK) successfully reduced the tumor uptake of both ⁶⁴Cu-DOTA-RGD tetramer and ⁶⁴Cu-DOTA-RGD tetramer uptake in the U87MG tumor, and reduced kidney uptake to the background level, confirming the integrin αᵥβ₃ binding specificity of both tracers in vivo.

### Discussion

This study described the synthesis of ⁶⁴Cu-labeled RGD tetramer and RGD octamer based on the RGDyK sequence and their use for PET imaging of tumor integrin αᵥβ₃ expression. These RGD multimers showed very high integrin αᵥβ₃ binding affinity and specificity as determined by cell adhesion assay and cell binding assay. The binding affinity and specificity of the newly developed tracers (⁶⁴Cu-DOTA-RGD tetramer and ⁶⁴Cu-DOTA-RGD octamer) in vivo was also confirmed by biodistribution studies and quantitative microPET imaging experiments.

A variety of radiolabeled peptides have been evaluated for tumor localization and therapy (Eur J Nucl Med Mol Imaging. 2007;34:267-273, Nucl Med Biol. 2007;34:29-35, J Nucl Med. 2005;46:1707-1718, Mol Pharm. 2006;3:472-487, Bioconjug Chem. 2001;12:624-629, Mol Imaging Biol. 2004;6:350-359, and Cancer Res. 2002;62:6146-6151, each of which is incorporated herein by reference for the corresponding discussion). Radiolabeled RGD peptides are of particular interest because they bind to integrin αᵥβ₃ which is overexpressed on newly formed blood vessels and cells of many common cancer types. However, most RGD peptide-based tracers developed so far have fast blood clearance accompanied by relatively low tumor uptake and rapid tumor washout, presumably due to the suboptimal receptor-binding affinity/selectivity and inadequate contact with the binding pocket located in the extracellular segment of integrin α_{vβ}₃.

We and others have previously applied the concept of bivalency to develop dimeric RGD peptides for tumor targeting (J Nucl Med. 2006;47:113-121 Bioconjug Chem. 2001;12:624-629, Mol Imaging Biol. 2004;6:350-359, Cancer Res. 2002;62:6146-6151, and Cancer Biother Radiopharm. 2004;19:399-404, each of which is incorporated herein by reference for the corresponding discussion). The introduction of the dimeric RGD peptide system resulted in higher receptor-binding affinity/specificity for integrin αᵥβ₃ in vitro and enhanced tumor uptake and retention in vivo than the RGD monomer. Recently, we reported that ⁶⁴Cu-labeled tetrameric RGDfK peptide had significantly high affinity and specificity than both the RGD dimer and the RGD monomer in the integrin αᵥβ₃-positive U87MG tumor model due to the synergistic effect of polyvalency (J Nucl Med. 2005;46:1707-1718, which is incorporated herein by reference for the corresponding discussion). Previously, we also found that replacing D-Phe (f) with D-Tyr (y) increased the hydrophilicity of the RGD peptides and resulted in increased integrin αᵥβ₃ mediated tumor uptake and more favorable biokinetics in an orthotopic MDA-MB-435 breast cancer model (Mol Imaging Biol. 2004;6:350-359, which is incorporated herein by reference for the corresponding discussion). Based on these findings and incremental improvement on tumor targeting and pharmacokinetics as compared with the previous RGD peptide analogs, we then devoted our efforts to the synthesis of tetrameric and octameric RGD peptides with repeating c(RGDyK) units connected through glutamate linkers.

With the RGD/integrin system, polyvalency has been shown to be able to significantly improve integrin binding affinity and selectivity (J Med Chem. 2006;49:2268-2275, which is incorporated herein by reference for the corresponding discussion). It is reported that the minimum linker length between the two RGD moieties should be about 3.5 nm (˜25 bond distances) for simultaneous integrin αᵥβ₃ binding in the immobilized integrin αᵥβ₃ assay (Radiochimica Acta. 2004;92:317-327, which is incorporated herein by reference for the corresponding discussion). For our RGD tetramer (E{E[c(RGDyK)]₂}₂ (FIG. 5-1A), the longest distance between the two RGD motifs is ˜30 bond lengths, long enough for simultaneous binding to adjacent integrin αᵥβ₃. For the RGD octamer, the distance is ˜40 bond lengths and simultaneous binding to two or more receptors is possible.

We employed two types of assays to examine the interaction between RGD multimers and αᵥβ₃ integrin. We first used cell adhesion assay to assess the anti-adhesion effect of the RGD multimers against integrin αᵥβ₃. The RGD octamer showed significantly enhanced inhibition ability than the monomer/dimer/tetramer counterparts which could be attributed to the multiple binding sites and/or significantly increased local concentration. To evaluate the effect of polyvalency, we calculated the “multivalent enhancement ratio (MVE)” which was obtained by dividing the IC₅₀ value for the RGD monomer by the IC₅₀ of the RGD multimer (J Med Chem. 2006;49:6087-6093, which is incorporated herein by reference for the corresponding discussion). The anti-adhesion MVE of the RGD tetramer and the RGD octamer was 8.4 and 25.6, respectively (Table 1, Example 4). We then carried out cell binding assay, an often-used method to determine the receptor binding affinity of a given ligand. Again, the integrin αᵥβ₃ binding affinity followed the order of RGD octamer>RGD tetramer>RGD dimer>RGD monomer (FIG. 5-2C and Table 1, Example 4). DOTA conjugation had minimal effect on the binding affinity of the RGD peptides. The receptor binding MVE for the RGD tetramer and the RGD octamer was calculated to be 5.9 and 20.3, respectively. Based on both cell adhesion assay and cell binding assay, RGD octamer showed stronger multivalent effect than the RGD tetramer.

When applied to the U87MG glioblastoma xenograft model which has been well established to have high integrin αᵥβ₃ expresion (J Nucl Med. 2005;46:1707-1718 and J Nucl Med. 2006;47:113-121, each of which is incorporated herein by reference for the corresponding discussion), ⁶⁴Cu-DOTA-RGD tetramer showed prominent tumor uptake and primarily renal clearance (FIG. 5-3A and FIG. 5-4A). ⁶⁴Cu-DOTA-RGD octamer had slightly higher initial tumor uptake and much longer tumor retention. The initial rapid and high tumor uptake might be attributed to the high integrin αᵥβ₃ binding affinity of both tracers. The larger molecular size of ⁶⁴Cu-DOTA-RGD octamer, along with the stronger MVE, may be attributed to its longer circulation time and slower tumor washout as compared to ⁶⁴Cu-DOTA-RGD tetramer. We also tested these two tracers in the c-neu oncomouse model. Both tracers showed significantly higher uptake in the spontaneous tumor (medium integrin expression) than the dimeric and monomeric analogs (data not shown). The difference between ⁶⁴Cu-DOTA-RGD tetramer and ⁶⁴Cu-DOTA-RGD octamer in this model is more substantial than in the U87MG xenograft model. The tumor uptake of ⁶⁴Cu-DOTA-RGD octamer was almost twice as high as that of ⁶⁴Cu-DOTA-RGD tetramer (FIG. 5-4B). Similar pattern is also observed in the orthotopic MDA-MB-435 (medium integrin expression) breast cancer model (data not shown). In the medium integrin αᵥβ₃ expressing tumor models (e.g., MDA-MB-435 and the c-neu oncomice), the advantage of higher integrin αᵥβ₃ binding affinity and selectivity of the RGD octamer over the RGD tetramer appears to be more obvious than in high integrin expressing tumor models (e.g., U87MG). The mechanism underlying such phenomenon remains to be elucidated.

Comparing with ⁶⁴Cu-DOTA-RGD tetramer, ⁶⁴Cu-DOTA-RGD octamer exhibited significantly higher renal uptake in both s.c. U87MG xenografts and the mammary adenocarcinoma-bearing c-neu oncomice. We initially proposed that the very high renal uptake of ⁶⁴Cu-DOTA-RGD octamer as compared to other RGD oligomers might be caused by the overall molecular charge difference. If we assign a value of −1 to each acidic residue (Asp (D) and Glu (E)) and the C-terminal —COOH, a value of +1 to each basic residue (Arg (R) and Lys (K)) and the N-terminal —NH₂, the overall charge of the peptide can be determined by adding up the charges. For both RGD tetramer and RGD octamer, the overall molecular charges are +1 although the RGD octamer has higher number of charged amino acid residues. Positively charged radio-labeled peptides or metabolites are usually retained in the kidney after resorption by renal tubular cells and lysosomal proteolysis. Blocking cationic binding sites in the kidneys with cationic amino acid infusion has been reported to reduce the renal uptake without compromising the tumor activity accumulation in both mice and humans (J Nucl Med. 2006;47:528-533, which is incorporated herein by reference for the corresponding discussion). We tried the blocking experiment for the ⁶⁴Cu-DOTA-RGD octamer by co-injecting excess amount of D-lysine, the kidney uptake was only marginally reduced suggesting that the overall molecular charge does not contribute significantly to the high renal uptake (data not shown).

We noticed that even though the kidney uptake of ⁶⁴Cu-DOTA-RGD octamer was high, there was no appreciable activity excreted to the urinary bladder over time. Such phenomenon suggests that there might be receptor mediated binding involved. Integrins play important roles in renal development and integrin αᵥβ₃, in particular, has been identified in many parts of the developing kidney. Integrin αᵥβ₃ is expressed in the renal endothelium in adults and, to a lesser extent, in all tubular epithelium (Curr Opin Nephrol Hypertens. 1999;8:9-14, which is incorporated herein by reference for the corresponding discussion). Effective blocking of activity accumulation in the kidney in the presence of excess amount of c(RGDyK) also confirmed the integrin αᵥβ₃ specificity of both ⁶⁴Cu-DOTA-RGD tetramer and ⁶⁴Cu-DOTA-RGD octamer (FIG. 5-2B & 5-5B). Immunohistochemical staining showed that the mouse kidneys have very high β₃ expression on endothelial cells of the small glomeruli vessels (FIG. 5-5C), which further confirms that the renal uptake of both tracers are integrin specific. The trend of increased kidney uptake from RGD monomer, dimer, tetramer, to octamer would thus be due, in part, to the increased αᵥβ₃ binding affinity and the molecular size.

It is of interest to have high tumor-to-kidney ratios as well as high absolute tumor uptake and longer retention for both imaging and therapeutic applications. For imaging purposes, the renal accumulation of radiolabeled peptides will reduce the detection sensitivity in the vicinity of the kidneys. For therapeutic applications, the renal accumulation of radiolabeled peptides limits the maximum tolerated doses that can be administered without the induction of radiation nephrotoxicity. Thus, further modification is needed to improve the pharmacokinetics of RGD peptide-based radiopharmaceuticals. First, high αᵥβ₃ binding affinity is needed to afford high tumor uptake and retention. For RGD octamer, the density of RGD units is rather high and not all RGD units are amenable to effective binding to integrin αᵥβ₃ located on the same cell surface. Our future work will focus on the structure-activity relationship study to develop various dendritic and polymeric scaffolds for attaching RGD peptides thereby further enhancing the multivalency effect. Second, appropriate modification of the DOTA-RGD multimers is needed to reduce the renal uptake. Inserting a bifunctional linker between the DOTA chelator and the RGD multimer as pharmacokinetic modifier may be able to modulate the overall molecular charge, hydrophilicity, and molecular size, thus may improve the in vivo pharmacokinetics without compromising the tumor targeting efficacy of the resulted radioconjugates.

### Conclusion

⁶⁴Cu-DOTA-RGD tetramer and ⁶⁴Cu-DOTA-RGD octamer were developed for PET imaging of tumor integrin αᵥβ₃ expression. The RGD octamer showed significantly higher integrin αᵥβ₃ binding affinity in vitro than the RGD tetramer. Based on the noninvasive microPET studies, both tracers showed rapid and high tumor uptake, slow washout rate, and good tumor-to-background contrast in the U87MG xenografts and the c-neu oncomice. Overall, polyvalency has a profound effect on the receptor binding affinity and in vivo kinetics of ⁶⁴Cu-DOTA-RGD multimers. The information obtained here may guide future development of integrin αᵥβ₃-targeted imaging and internal radiotherapy agents. These RGD peptide-based radiopharmaceuticals may also have promising applications in other angiogenesis related diseases such as rheumatoid arthritis, myocardial infarction, and stroke.

### Example 5

### Introduction

In this Example, we coupled multimeric RGD peptides with 1,4,7-triazacyclononanetriacetic acid (NOTA) and labeled the NOTA-RGD conjugates with ⁶⁸Ga for quantitative PET imaging studies.

Three cyclic RGD peptides, c(RGDyK) (RGD1), E[c(RGDyK)]₂ (RGD2), and E{E[c(RGDyK)]₂}₂ (RGD4), were conjugated with macrocyclic chelator 1,4,7-triazacyclononane-1,4,7-triacetic acid (NOTA) and labeled with ⁶⁸Ga. Integrin affinity and specificity of the peptide conjugates were assessed by cell based receptor binding assay and the tumor targeting efficacy of ⁶⁸Ga-labeled RGD peptides was evaluated in a subcutaneous U87MG glioblastoma xenograft model.

U87MG cell based receptor binding assay using ¹²⁵I-echistatin as radioligand showed that integrin affinity followed the order of NOTA-RGD4>NOTA-RGD2>NOTA-RGD1. All three NOTA conjugates allowed nearly quantitative ⁶⁸Ga-labeling within 10 min. Quantitative microPET imaging studies showed that ⁶⁸Ga-NOTA-RGD4 had the highest tumor uptake but also prominent activity accumulation in the kidneys. ⁶⁸Ga-NOTA-RGD2 had higher tumor uptake (e.g. 2.80±0.11% ID/g at 1 h p.i.) and similar pharmacokinetics (4.42±0.39 tumor/muscle ratio, 2.04±0.05 tumor/liver ratio, and 1.11±0.13 tumor/kidney ratio) compared with ⁶⁸Ga-NOTA-RGD1.

The dimeric RGD peptide tracer ⁶⁸Ga-NOTA-RGD2 with good tumor uptake and favorable pharmacokinetics warrants further investigation for potential clinical translation to image integrin αᵥβ₃.

### Materials and Methods

All commercially obtained chemicals were of analytical grade and used without further purification. S-2-(4-Isothiocyanatobenzyl)-1,4,7-triazacyclononane-1,4,7-triacetic acid (p-SCN-Bn-NOTA) was purchased from Macrocyclics, Inc. Cyclic RGD peptides c(RGDyK) (denoted as RGD1) and E[c(RGDyK)]₂ (denoted as RGD2) were from Peptides International, Inc. Tetrameric RGD peptides E{E[c(RGDyK)]₂}₂ (denoted as RGD4) were synthesized as previously described (J Nucl Med. 2007;48:1162-71, which is incorporated herein by reference for the corresponding discussion). ⁶⁸Ga was obtained from a ⁶⁸Ge/⁶⁸Ga generator (produced by Cyclotron, Obninsk, Russia) eluted with 4 mL of 0.1 N HCl. The semi-preparative reversed-phase HPLC system was the same as previously reported (J Nucl Med. 2006;47:113-21, which is incorporated herein by reference for the corresponding discussion) with a flow rate of 5 mL/min. The mobile phase was changed from 95% solvent A (0.1% trifluoroacetic acid [TFA] in water) and 5% solvent B (0.1% TFA in acetonitrile, ACN) (0-2 min) to 35% solvent A and 65% solvent B at 32 min. Analytical HPLC has the same gradient system except that a Vydac 218TP54 column (5 μm, 250×4.6 mm) was used and the flow rate was 1 mL/min. The UV absorbance was monitored at 218 nm and the identification of the peptides was confirmed based on the UV spectrum acquired using a PDA detector.

### Synthesis of NOTA Conjugated Multimeric RGD Peptides

NOTA-RGD conjugates were prepared under standard SCN-amine reaction condition. In brief, a solution of 2 μmol RGD peptide (monomer, dimer, or tetramer) was mixed with 6 μmol p-SCN-Bn-NOTA in sodium bicarbonate buffer (pH=9.0). After stirring at room temperature for 5 h, the NOTA conjugated RGD peptides were isolated by semi-preparative HPLC. The collected fraction was combined and lyophilized to afford the final product as a white powder. NOTA-c(RGDyK) (NOTA-RGD1) was obtained in 61% yield with 13.4 min retention time on analytical HPLC. Matrix-assisted laser desorption/ionization (MALDI) time-of-light (TOF) mass spectrometry (MS): m/z 1070.4 for [MH]⁺ (C₄₇H₆₈N₁₃O₄S, calculated molecular weight 1070.5). NOTA-E[c(RGDyK)]₂ (NOTA-RGD2) was obtained in 52% yield with 14.1 min retention time on analytical HPLC. MALDI-TOF-MS: m/z 1800.2 for [MH]⁺ (C₇₉H₁₁₄N₂₃O₂₄S, calculated molecular weight 1800.8). NOTA-E{E[c(RGDyK)]₂}₂ (NOTA-RGD4) was obtained in 43% yield with 14.6 min retention time on analytical HPLC. MALDI-TOF-MS: m/z 3266.6 for [MH]⁺ (C₁₄₃H₂₀₆N₄₃O₄₄S, calculated molecular weight 3263.5).

### Radiochemistry

The ⁶⁸Ga labeling procedure was conducted according to the methods previously described (Eur J Nucl Med. 2000;27:273-82, which is incorporated herein by reference for the corresponding discussion). Briefly, 10 nmol of NOTA-RGD peptides were dissolved in 500 μL of 0.1 M sodium acetate buffer and incubated with 185 MBq of ⁶⁸Ga for 10 min at 40° C. ⁶⁸Ga-NOTA-RGD peptides were then purified by semi-preparative HPLC, and the radioactive peak containing the desired product was collected. After removal of the solvent by rotary evaporation, the residue was reconstituted in 800 μL of phosphate-buffered saline for further in vitro and in vivo experiments. The labeling was done with 90% decay-corrected yield for NOTA-RGD1 (retention time (Rₜ)=12.9 min), 82% for NOTA-RGD2 (Rₜ=13.8 min), and 64% for NOTA-RGD4 (Rₜ=14.4 min).

### Cell Line and Animal Model

Human glioblastoma U87MG cells were grown in Dulbecco's medium (Gibco) supplemented with 10% fetal bovine serum (FBS), 100 IU/mL penicillin, and 100 μg/mL streptomycin (Invitrogen Co.), at 37° C. in a humidified atmosphere containing 5% CO₂. All animal experiments were performed under a protocol approved by Stanford's Administrative Panel on Laboratory Animal Care (APLAC). The U87MG tumor model was generated by subcutaneous injections of 5×10⁶ cells in 100 μL of PBS into the front legs of female athymic nude mice (Harlan, Indianapolis, Ind.). The mice were subjected to microPET studies when the tumor volume reached 100-300 mm³ (3-4 weeks after inoculation) (J Nucl Med. 2007;48:1536-44 and J Nucl Med. 2007;48:1162-71, which is incorporated herein by reference for the corresponding discussion).

### Cell Binding Assay

In vitro integrin αᵥβ₃-binding affinity and specificity of NOTA-RGD1, NOTA-RGD2 and NOTA-RGD4 were assessed via competitive cell binding assay using ¹²⁵I-echistatin as the integrin αᵥβ₃-specific radioligand (J Nucl Med. 2005;46:1707-18, which is incorporated herein by reference for the corresponding discussion). The best-fit 50% inhibitory concentration (IC₅₀) values for the U87MG cells were calculated by fitting the data with nonlinear regression using Graph-Pad Prism (GraphPad Software, Inc.) and compared to that of monomeric RGD peptide c(RGDyK) (RGD1).

microPET Imaging

PET scans and image analysis were performed using a microPET R4 rodent model scanner (Siemens Medical Solutions) as previously reported (J Nucl Med. 2006;47:113-21 and J Nucl Med. 2005;46:1707-18, which is incorporated herein by reference for the corresponding discussion). MicroPET studies were performed by tail-vein injection of about 3.7 MBq of ⁶⁸Ga-NOTA-RGD1, ⁶⁸Ga-NOTA-RGD2 or ⁶⁸Ga-NOTA-RGD4 under isoflurane anesthesia. The 60-min dynamic scan (5×1 min, 10×3 min, 5×5 min, total of 20 frames) was started 1 min after injection. A 2 h time point static scan was also acquired after the 60 min dynamic scan. Five min static PET images were also acquired separately at 30 min, 1 h and 2 h time points post-injection (p.i.) for another set of tumor-bearing mice (n=3/tracer). The images were reconstructed by a 2-dimensional ordered-subsets expectation maximum (OSEM) algorithm and no correction was necessary for attenuation or scatter correction. For blocking experiment, a mouse bearing a U87MG tumor were co-injected with 10 mg/kg mouse body weight of c(RGDyK) and 3.7 MBq of ⁶⁸Ga-NOTA-RGD2. Five min static PET scan was then acquired at 1 h p.i. (n=3).

### Biodistribution Studies

Female nude mice bearing U87MG xenografts were injected with 3.7 MBq of ⁶⁸Ga-NOTA-RGD2 to evaluate the distribution of these tracers in the major organs of mice. A blocking experiment was also performed by coinjecting radiotracer with a saturating dose of c(RGDyK) (10 mg/kg of mouse body weight). All mice were sacrificed and dissected at 1 h after injection of the tracer. Blood, tumor, major organs and tissues were collected and wet weighed. The radioactivity in the tissue was measured using a γ-counter (Packard). The results were presented as percentage injected dose per gram of tissue (% ID/g). For each mouse, the radioactivity of the tissue samples was calibrated against a known aliquot of the injectate and normalized to a body mass of 20 g. Values were expressed as mean±SD for a group of 3 animals.

### Statistical Analysis

Quantitative data were expressed as mean±SD. Means were compared using One-way ANOVA and student's t-test. P values<0.05 were considered statistically significant.

### Results

### Chemistry and Radiochemistry

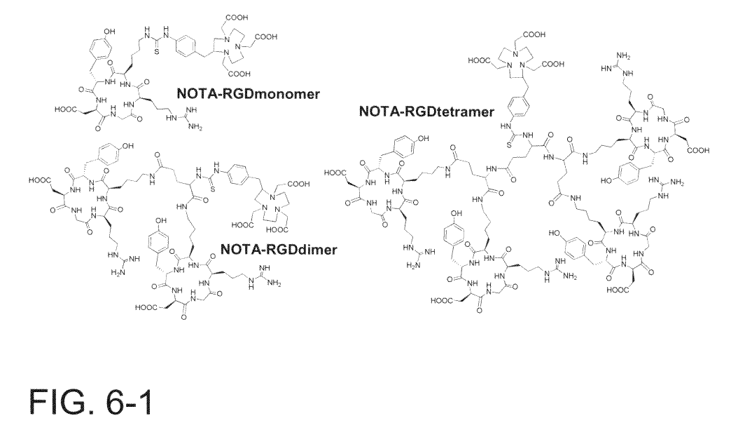

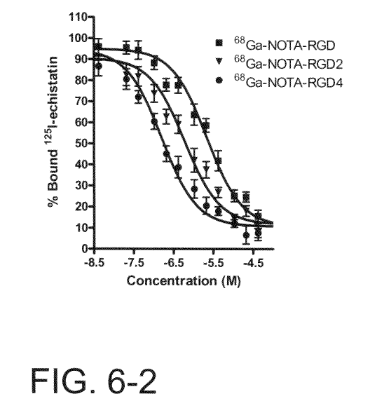

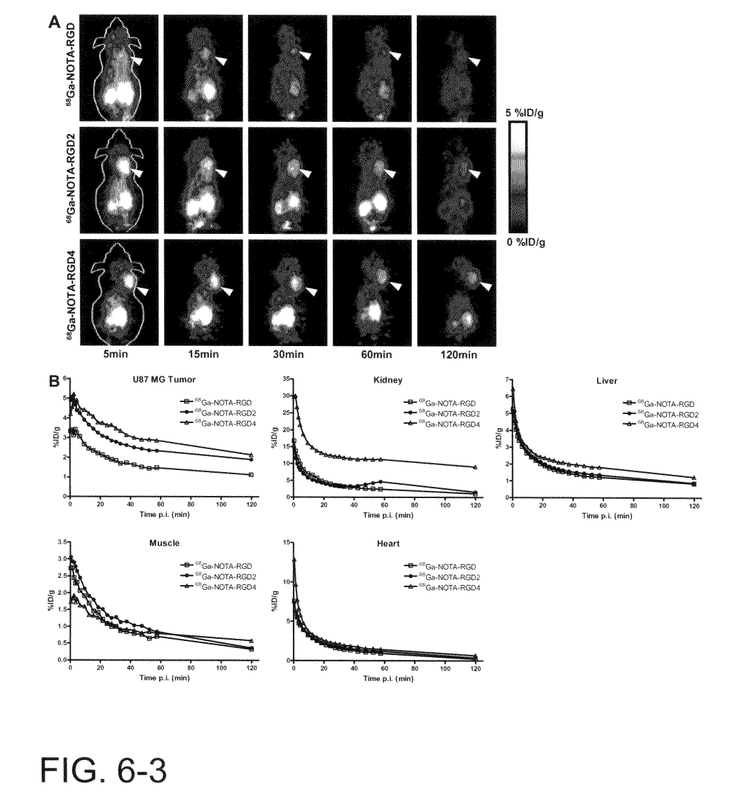

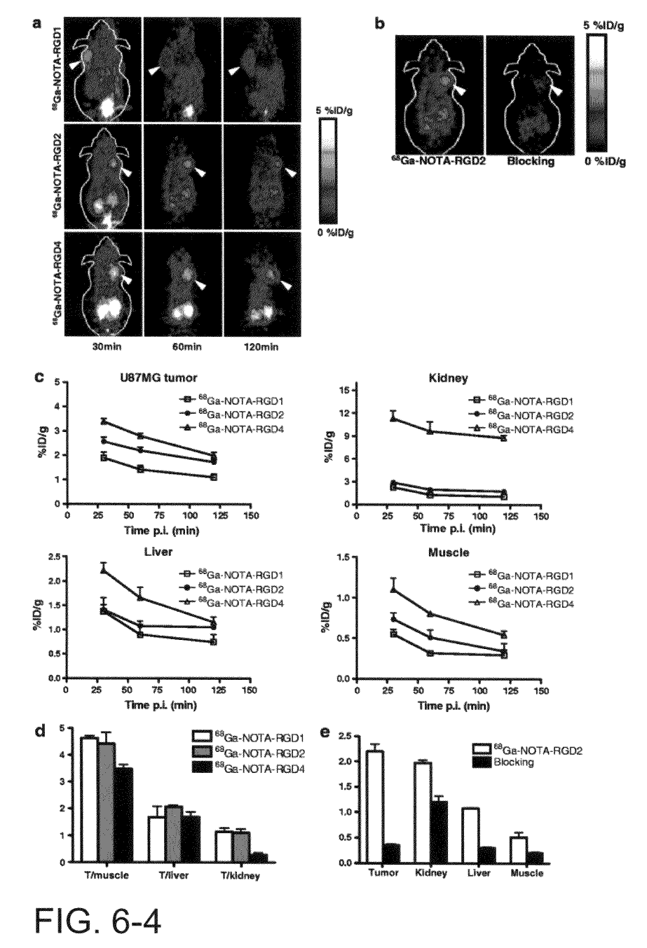

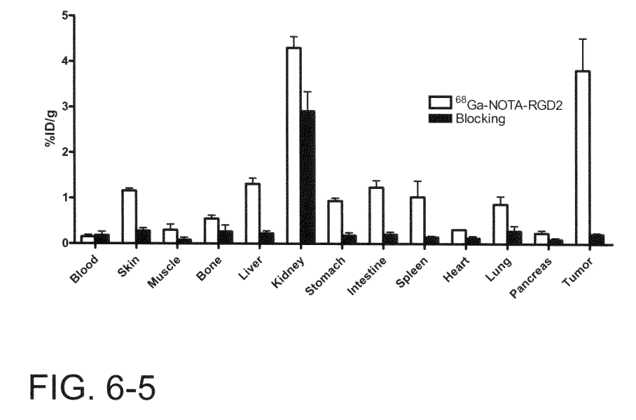

The NOTA-RGD conjugates were prepared from RGD peptides and p-SCN-Bn-NOTA in moderate yields (FIG. 6-1). Both HPLC and mass spectroscopy were used to confirm the identity of the products. ⁶⁸Ga was eluted from the ⁶⁸Ge/⁶⁸Ga generator and used directly for the reaction after adjusting the pH. On the analytical HPLC, a slightly decreased retention time was observed between ⁶⁸Ga-NOTA-RGD multimers and the unlabeled conjugates (0.5 min for monomer, 0.3 min for dimer and 0.2 min for tetramer conjugates). The labeling was done within 10 min with a decay corrected yield ranging from 64% to 90% and a radiochemical purity of more than 98%. The specific activity of purified ⁶⁸Ga-NOTA-RGD multimers was about 9.7-13.6 MBq/nmol.

### Cell Binding Assay

We compared the receptor-binding affinity of NOTA-RGD1, NOTA-RGD2 and NOTA-RGD4 using a competitive cell binding assay method (FIG. 6-2). All three peptide conjugates inhibited the binding of ¹²⁵I-echistatin (integrin αᵥβ₃ specific) to U87MG cells in a concentration dependent manner. The IC₅₀ values for NOTA-RGD1, NOTA-RGD2 and NOTA-RGD4 were 218±28, 60.1±7.6 and 16.1±3.1 nmol/L (n=3), respectively. The comparable IC₅₀ values of NOTA-RGD1 and c(RGDyK) (IC₅₀ was determined to be 189 nmol/L under the same condition, data not shown) suggest that incorporation of the NOTA motif had a minimal effect on the receptor binding avidity. Due to the polyvalency effect, NOTA-RGD2 had 3-fold higher integrin αᵥβ₃ affinity than NOTA-RGD1, and NOTA-RGD4 further increased the integrin avidity by another 3-fold as compared to NOTA-RGD2 (or 13-fold higher affinity than NOTA-RGD1). Note that the IC₅₀ values measured from cell-based integrin binding assay are typically lower than those obtained from purified αᵥβ₃ integrin protein fixed on a solid matrix (e.g., an ELISA and solid-phase receptor binding assay) (J Nucl Med. 2001;42:326-36, which is incorporated herein by reference for the corresponding discussion).

microPET Imaging Study

The tumor-targeting efficacy of ⁶⁸Ga-NOTA-RGD probes in U87MG tumor-bearing nude mice was first evaluated by 1 h dynamic microPET scans followed by a static scan at 2 h p.i. Representative decay-corrected coronal images at different time points after injection are shown in FIG. 6-3A. The U87MG tumors were clearly visualized with good tumor-to-background contrast for all three tracers. For ⁶⁸Ga-NOTA-RGD1, the tumor uptake was 3.24, 2.35,1.84, 1.47, and 1.12% ID/g at 5, 15, 30, 60, and 120 min, respectively. For ⁶⁸Ga-NOTA-RGD2, the tumor uptake was 4.39, 3.46, 2.79, 2.34, and 1.89 % ID/g at 5, 15, 30, 60, and 120 min respectively. For ⁶⁸Ga-NOTA-RGD4, the tumor uptake was 4.90, 4.08, 3.48, 2.86, and 2.13% ID/g at 5, 15, 30, 60, and 120 min respectively (FIG. 6-3B). All three tracers were excreted mainly through the kidneys. The renal uptake of ⁶⁸Ga-NOTA-RGD and ⁶⁸Ga-NOTA-RGD2 had no significant difference (P>0.05). Although ⁶⁸Ga-NOTA-RGD4 had the highest tumor uptake, the uptake in the kidneys was almost doubled compared with those of the monomeric and dimeric analogs (P<0.001). All three compounds have comparable liver and muscle uptake in the dynamic scan. ⁶⁸Ga-NOTA-RGD4 exhibited the highest heart uptake at the early time point (data not shown), which might indicate the longer circulation time of this tracer. However, this difference was diminished at later time points.

To assess the effect of the anesthesia on the clearance of the tracers from the nontargeted tissues (such as the liver and kidneys), we also performed separate static scans at 30, 60, and 120 min (n=3) in addition to the above dynamic scans. From FIG. 6-4a, it can be seen that all tracers gave much better tumor-to-background contrast than from dynamic scans due to the faster clearance of nonspecifically bound activity when the rodents were kept awake vs under isoflurane anesthesia. The tumor uptake was determined to be 1.9±0.2, 1.4±0.2, and 1.1±0.1% ID/g at 30, 60, and 120 min for ⁶⁸Ga-NOTA-RGD1; 2.6±0.2, 2.2±0.1, and 1.7±0.1% ID/g at 30, 60, and 120 min for ⁶⁸Ga-NOTA-RGD2; and 3.4±0.1, 2.8±0.1, and 2.0±0.2% ID/g at 30, 60 and 120 min for ⁶⁸Ga-NOTA-RGD4 (FIG. 6-4c). Compared with the dynamic scans, these uptakes were only marginally decreased. In contrast, the kidney uptake measured from the region of interest (ROI) analysis of the static scans was significantly lower than that from the dynamic scans at all time points examined. For example, ⁶⁸Ga-NOTA-RGD2 exhibited only 2.0% ID/g kidney uptake in this static scan compared with 4.6% ID/g in the dynamic scan at 1 h p.i. ⁶⁸Ga-NOTA-RGD4 showed the highest liver uptake among the three RGD probes tested, which might be attributed to its relatively large molecular size. The nonspecific uptake in the muscle was at a very low level for all three tracers. We also calculated the tumor-to-major-organ ratios of these ⁶⁸Ga-NOTA-RGD probes to compare their tumor targeting efficacy and in vivo pharmacokinetics at 1 h p.i. (FIG. 6-4d). Although ⁶⁸Ga-NOTA-RGD4 had the highest tumor uptake, the tumor-to-kidney ratio was significantly lower than that of ⁶⁸Ga-NOTA-RGD1 and ⁶⁸Ga-NOTA-RGD2. Comparable tumor/liver, tumor/kidney, and tumor/muscle ratios were observed for ⁶⁸Ga-NOTA-RGD1 and ⁶⁸Ga-NOTA-RGD2, while the absolute tumor uptake of ⁶⁸Ga-NOTA-RGD2 was significantly higher than that of ⁶⁸Ga-NOTA-RGD1 (P<0.01). Taken together,⁶⁸Ga-NOTA-RGD2 provided the best image quality with the same amount of injected activity among the three tracers tested. The microPET images at 1 h p.i. of U87MG tumor-bearing mouse injected with ⁶⁸Ga-NOTA-RGD2 and a blocking dose of c(RGDyK) are shown in FIG. 6-4b. The U87MG tumor uptake was reduced to the background level (0.31±0.02% ID/g), confirming the integrin αᵥβ₃-specific binding of ⁶⁸Ga-NOTA-RGD2 in the tumor. Similar to the previously observed results, the tracer cleared from the body significantly faster and the uptake in most of the organs (e.g., liver, kidneys, and muscle) was also lower than those without c(RGDyK) blocking (FIG. 6-4e).

### Biodistribution Studies

To validate the accuracy of microPET quantification, we also performed a biodistribution experiment by using the direct tissue sampling technique. For this, U87MG tumor-bearing mice were tail-vein injected with ⁶⁸Ga-NOTA-RGD2 (typically 740 Bq/mouse) and sacrificed at 1 h p.i. The data shown as the percentage administered activity (injected dose) per gram of tissue (% ID/g) in FIG. 6-5. The tumor uptake was 3.82±0.7% ID/g and the kidney uptake was 4.30±0.25% ID/g for the control group. The uptake values in the other major organs were around or less than 1% ID/g.

To confirm the receptor specificity, ⁶⁸Ga-NOTA-RGD2 was co-injected with blocking dose of c(RGDyK) (10 mg/kg). A decrease of radioactivity was seen in all dissected tissues and organs (FIG. 6-5), with the change of tumor uptake being the most significant, as it was reduced markedly from 3.82±0.7 to 0.21±0.03% ID/g at 1 h time point. Similar patterns have been observed in other radiolabeled RGD peptide studies as well.

### Discussion

The development of radiolabeled peptides for diagnostic and therapeutic applications has expanded exponentially in the last decade. Peptidic radiopharmaceuticals can be produced easily and inexpensively and have many favorable properties, including fast clearance, rapid tissue penetration, low antigenecity (Mol Pharm. 2006;3:472-87 and BioDrugs. 2004;18:279-95, which is incorporated herein by reference for the corresponding discussion). We are particularly interested in developing radiolabeled RGD peptides because they bind to integrin αᵥβ₃ that is overexpressed on newly formed neovasculature and the tumor cells of many common cancer types. We and others also have found that multimeric RGD peptides can significantly enhance the affinity of the receptor-ligand interaction through the polyvalency effect. In this study we explored the imaging characteristics of ⁶⁸Ga-labeled RGD multimers and sought to identify an optimal peptide conjugate for this generator-based short-lived PET isotope.

Both NOTA and DOTA can be used as bifunctional chelators for ⁶⁸Ga labeling. However, DOTA has a larger cavity than NOTA, which results in lower stability of the ⁶⁸Ga complex. The log stability constants for Ga-NOTA was determined to be 30.98, compared with 21.33 for Ga-DOTA complex (Inorganica Chimica Acta. 1991;190:37-46 and Inorganica Chimica Acta. 1991;181:273-80, which is incorporated herein by reference for the corresponding discussion). Moreover, the ⁶⁸Ga labeling of NOTA complex can be carried out at room temperature within short time, while the DOTA complex needs a much higher temperature and its application for protein or antibody labeling is thereby limited. Therefore, in this study, we constructed NOTA conjugated monomeric, dimeric and tetrameric RGD peptides for ⁶⁸Ga labeling. To examine the interaction between NOTA-RGDmultimers and integrin αᵥβ₃, we performed a cell-binding assay to assess the receptor-binding affinity of these ligands. The integrin αᵥβ₃-binding affinity followed the order of NOTA-RGD4>NOTA-RGD2>NOTA-RGD1. On the basis of the cell binding assay, we observed a multivalent effect for these RGD multimers.

After labeling with ⁶⁸Ga, we first performed dynamic scans for these tracers in the U87MG glioblastoma xenograft model, which has been well established to have a high integrin αᵥβ₃ expression. All three tracers showed prominent uptake in the tumor and predominant renal clearance. ⁶⁸Ga-NOTA-RGD4 had the highest tumor uptake, followed by ⁶⁸Ga-NOTA-RGD2 and ⁶⁸Ga-NOTA-RGD1. However ⁶⁸Ga-NOTA-RGD4 also exhibited much higher kidney uptake than monomeric and dimeric analog, which might limit its potential applications. We have previously shown that a high affinity RGD peptide ligand tends to accumulate in the kidney through both receptor-mediated binding and renal clearance. Rodent kidneys have been found to express integrin in the endothelial cells of small glomerulus vessels.

Radiometallic PET isotope ⁶⁸Ga has several distinct advantages over ⁶⁴Cu. First, the generator-based ⁶⁸Ga is more readily available than the cyclotron-produced ⁶⁴Cu. Second, ⁶⁸Ga possesses much higher positron efficiency (89%) than ⁶⁴Cu (17.4%). Third, Ga-NOTA complex is a highly stable complex, resulting in little transchelation when ⁶⁸Ga-labeled NOTA-peptide conjugates are administered intravenously. By contrast, 6⁴Cu complexes through DOTA or other macrocyclic ligand chelation are not necessarily stable enough to resist transchelation in the liver, creating an unnecessarily high hepatic uptake of ⁶⁴Cu. Indeed, ⁶⁸Ga-NOTA-RGD complexes show significantly lower liver uptake than ⁶⁴Cu-DOTA-RGD analogs.

Nevertheless, the relatively short half-life of ⁶⁸Ga (t_{1/2}=68 min) is a major concern for large sized peptides. Our previous data have shown that ⁶⁴Cu-DOTA-RDG4 is superior to the dimeric and monomeric RGD counterparts in terms of both tumor uptake and tumor/background contrast when most of the non-specific uptake has been cleared within 2-4 hours. Although ⁶⁸Ga-NOTA-RGD4 had significantly higher tumor uptake than ⁶⁸Ga-NOTA-RGD2 and ⁶⁸Ga-NOTA-RGD1, ⁶⁸Ga-labeled RGD tetramer also showed relatively high renal uptake so the tumor/kidney ratio of the tetramer was less than that of dimer and monomer. It is possible that at time points later than 2 h p.i. there would be sufficient renal clearance of ⁶⁸Ga-NOTA-RGD4 to improve the tumor/kidney ratio, but the relatively short half-life of ⁶⁸Ga might not allow visualization by microPET at time points beyond 2 h.

Despite the high receptor affinity of the tetrameric RGD peptide, the relatively large molecular size and consequently slow clearance of this peptide tracer makes it less suitable for ⁶⁸Ga-labeling and PET imaging as compared with the RGD monomer and dimer. As shown in FIG. 6-4, ⁶⁸Ga-NOTA-RGD2 and ⁶⁸Ga-NOTA-RGD1 had a comparable tumor to major organ ratio, but the absolute tumor uptake of the dimer is about twice as much as that of the monomer, thus providing better imaging quality. Therefore, we focused mainly on this dimeric tracer in the following experiments. The integrin αᵥβ₃ specificity of ⁶⁸Ga-NOTA-RGD2 was confirmed by effective tumor uptake inhibition in the presence of c(RGDyK) in both non-invasive PET imaging and biodistribution studies. It is also of note that the kidney uptake under dynamic scan (FIG. 6-3B) was significantly higher than that obtained under static scan (FIG. 6-4C). This is likely due to the reduced glomerular filtration rate of isoflurane anesthetized mice over conscious mice.

Through the comparison of tumor uptake and contrast among the three peptide tracers developed in this, we believe that ⁶⁸Ga-NOTA-RGD2 is a most promising tracer for further studies. Our future work on the ⁶⁸Ga-labeled dimeric RGD peptide tracer will be to test whether the tumor/background ratio derived from microPET imaging or direct tissue sampling reflects the tumor integrin expression level. Predominant renal clearance of ⁶⁸Ga-labeled RGD peptides will limit their applications in detecting lesions that are in the kidneys and around urinary bladder. Ways to reduce or eliminate renal clearance may be needed to image urological malignancies. A more thorough comparison between ⁶⁸Ga-labeled RGD peptides and other PET isotope (such as ¹⁸F and ⁶⁴Cu) labeled same peptides is also needed to determine the pros and cons of each radiotracer.

### Conclusion

Monomeric, dimeric and tetrameric RGD peptides have been labeled with the generator-produced ⁶⁸Ga for PET imaging of tumor integrin αᵥβ₃ expression. The short half-life of ⁶⁸Ga is highly compatible with the fast tumor localization of RGD peptides. Despite the fact that ⁶⁸Ga-NOTA-RGD4 has the highest integrin affinity in vitro and highest tumor uptake in vivo, its poor tumor/kidney ratio makes this tracer less useful than ⁶⁸Ga-NOTA-RGD1 and ⁶⁸Ga-NOTA-RGD2. ⁶⁸Ga-NOTA-RGD1 and ⁶⁸Ga-NOTA-RGD2 showed similar tumor-to-background contrast, but the dimer had higher tumor uptake and prolonged retention than the monomeric counterpart. In short, ⁶⁸Ga-NOTA-RGD2 may enable the production of kit-formulated PET radiopharmaceutical for integrin αᵥβ₃ imaging.

### Example 6

### Introduction

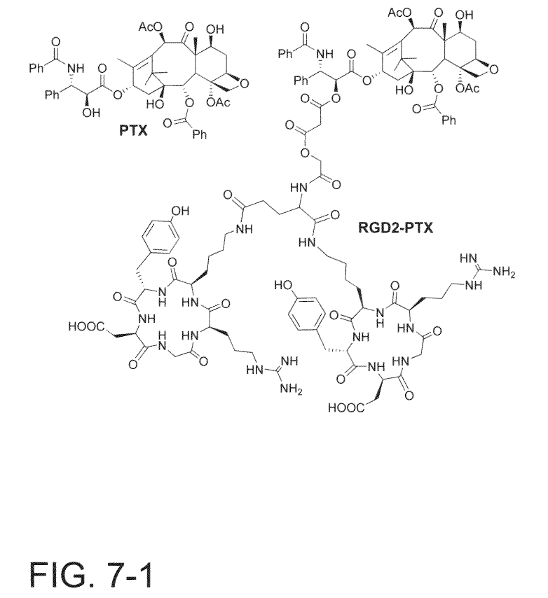

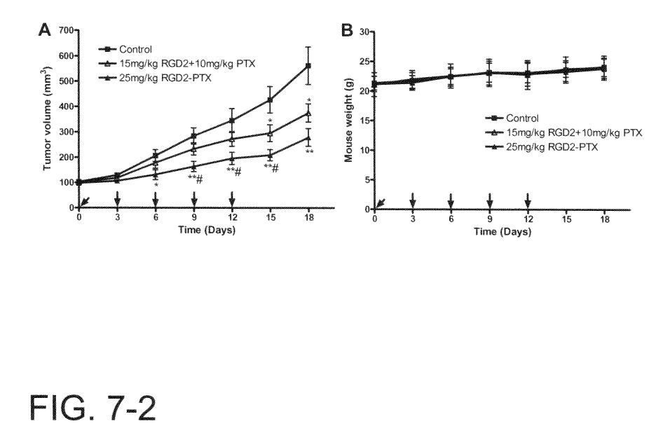

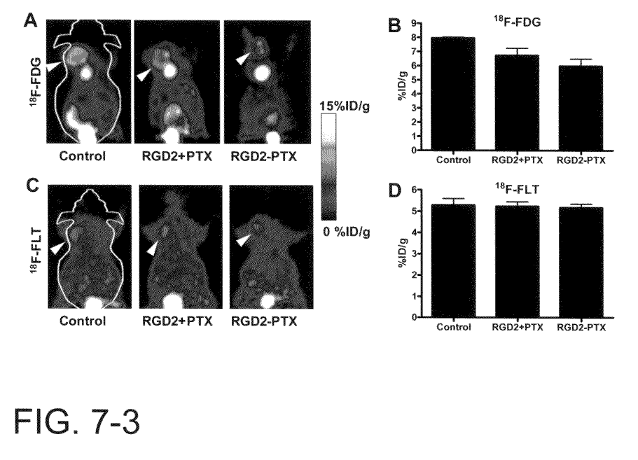

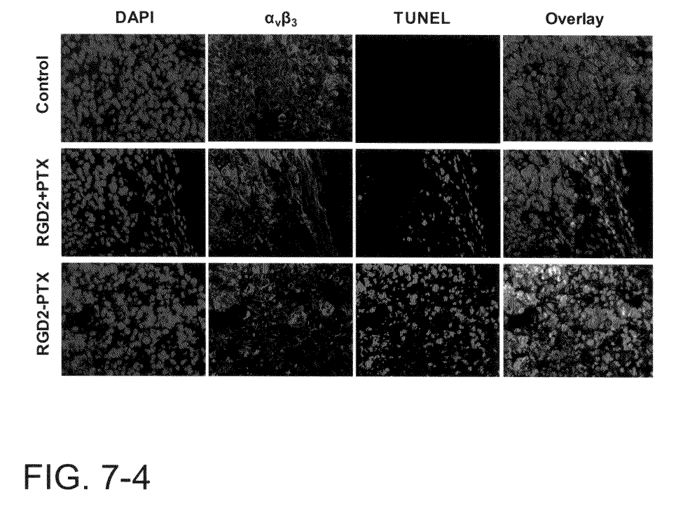

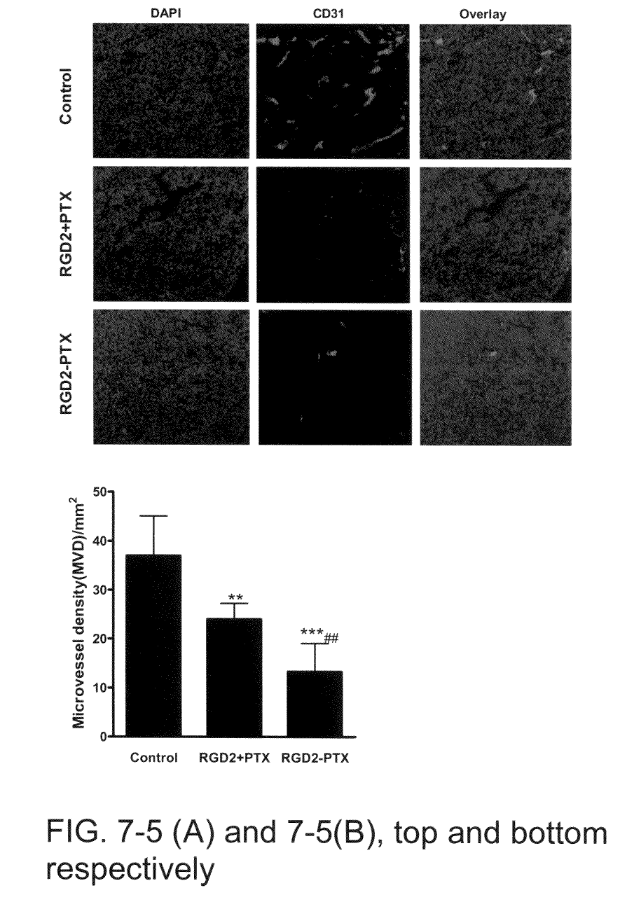

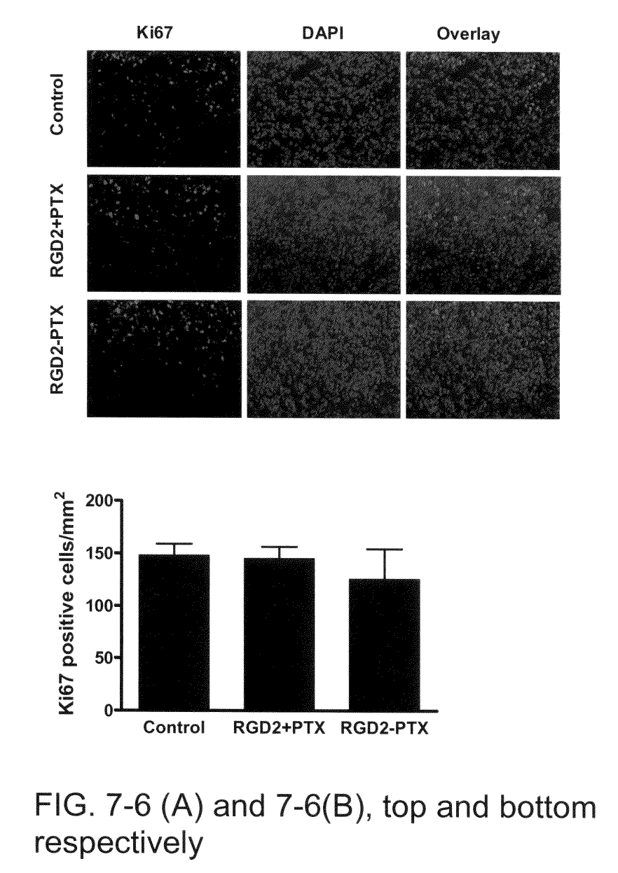

In this Example, we evaluated the antitumor efficacy of a dimeric RGD peptide paclitaxel conjugate (RGD2−PTX) in an orthotopic MDA-MB-435 breast cancer model. We have previously conjugated PTX with a dimeric RGD peptide E[c(RGDyK)]₂ (FIG. 7-1) and evaluated the antitumor activity in a metastatic breast cancer cell line MDA-MB-435 (J Med Chem. 2005;48:1098-106, which is incorporated herein by reference of the corresponding discussion). The in vitro results showed that the RGD2−PTX conjugate inhibited cell proliferation with activity comparable to that observed for paclitaxel, both of which were mediated by an arrest of G2/M-phase of the cell cycle followed by apoptosis. In addition, when RGD2−PTX was labeled with ¹²⁵I through the tyrosine residue on the RGD peptide, integrin specific accumulation of ¹²⁵I-RGD2−PTX in orthotopic MDA-MB-435 tumor was observed. Here we would like to extend this effort and study the anti-tumor effect of RGD2−PTX in vivo.

To assess the effect of conjugation and the presence of drug moiety on the MDA-MB-435 tumor and normal tissue uptake, the biodistribution of ³H-RGD2−PTX was compared with that of ³H-PTX. The treatment effect of RGD2−PTX and RGD2+PTX was measured by tumor size, ¹⁸F-FDG/PET, ¹⁸F-FLT/PET, and postmortem histopathology.

By comparing the biodistribution of ³H-RGD2−PTX and ³H-PTX we found that ³H-RGD2−PTX had higher initial tumor exposure dose and prolonged tumor retention than ³H-PTX. Metronomic low dose treatment of breast cancer indicated that RGD2−PTX is significantly more effective than PTX+RGD2 combination and solvent control. Although in vivo ¹⁸F-FLT/PET imaging and ex vivo Ki67 staining indicated little effect of the PTX based drug on cell proliferation, ¹⁸F-FDG/PET imaging showed significantly reduced tumor metabolism in the RGD2−PTX treated mice versus those treated with RGD2+PTX and solvent control. TUNEL staining also showed that RGD2−PTX treatment also had significantly higher cell apoptosis ratio than the other two groups. Moreover, the microvessel density was significantly reduced after RGD2−PTX treatment as determined by CD31 staining.

Our results demonstrate that integrin targeted delivery of paclitaxel allows preferential cytotoxicity to integrin expressing tumor cells and tumor vasculature. The targeted delivery strategies developed here may also be applied to other chemotherapeutics for selective tumor killing.

### Materials and Methods

All reagents, unless otherwise specified, were of analytical grade and purchased commercially. Dimeric RGD peptide E[c(RGDyK)]₂ was synthesized by Peptides International, Inc (Louisville, Ky.). PTX-2′-succinate (PTXSX) was prepared by reacting PTX (Hande Tech, Houston, Tex.) with equal molar amount of succinic anhydride in pyridine (J Med Chem. 1989;32:788-92, which is incorporated herein by reference for the corresponding discussion). ³H-PTX was purchased from Moravek Biochemicals, Inc. (Brea, Calif.) with a specific activity of 2.4 Ci/mmol.

### Preparation of RGD2−PTX and ³H-RGD2−PTX Conjugate

RGD2−PTX was prepared from dimeric RGD peptide E[c(RGDyK)]₂ according to our previously reported procedure (J Med Chem. 2005;48:1098-106, which is incorporated herein by reference for the corresponding discussion). ³H-RGD2−PTX was also obtained by using the same method. In brief, ³H-PTX was mixed with excess amount of non-radioactive PTX and reacted with succinate anhydride to provide carrier added ³H-PTXSX. The active ester ³H-PTXSX-OSSu was then prepared in situ and added to a solution of dimeric RGD peptide. The reaction mixture was incubated at 4° C. for overnight and then purified by semi-preparative reversed-phase high-performance liquid chromatography (RP-HPLC) on a Dionex 680 chromatography system with a UVD 170U absorbance detector. After lyophilization, ³H-RGD2−PTX conjugate was obtained as white fluffy powder in 48% yield with specific activity of 1.68 μCi/mg.

### Animal Model

All animal experiments were performed in compliance with the guidelines for the care and use of research animals established by the Stanford University's Animal Studies Committee. Female athymic nude mice (nu/nu) were obtained from Harlan (Indianapolis, Ind.) at 6-8 weeks of age and were kept under sterile conditions. The MDA-MB-435 cells were harvested and suspended in sterile PBS at a concentration of 5×10⁷ cells/mL. Viable cells (5×10⁶) in PBS (100 μL) were injected orthotopically in the right mammary fat pad. Palpable tumors appeared by day 10-14 post-implantation. Tumor growth was followed by caliper measurements of perpendicular measures of the tumor. The tumor volume was estimated by the formula: tumor volume=a×(b²)/2, where a and b are the tumor length and width respectively in mm.

### Biodistribution

To assess the effect of conjugation and the presence of drug moiety on the MDA-MB-435 tumor and normal tissue uptake, the biodistribution of ³H-RGD2−PTX was compared with that of ³H-PTX. Orthotopic MDA-MB-435 tumor-bearing female athymic nude mice (n=3 per time point) were injected with 2.9 μmol/kg ³H-RGD2−PTX or ³H-PTX via the tail vein. The animals were euthanized at 4, 24 and 48 h post injection and major organs and tissues were collected correspondingly. Approximately 100 mg of the tissue was added to glass scintillation vials containing 1 mL of tissue solubilizer SoluEne®-350 (Perkin-Elmer, Waltham, Mass.). These samples were digested at 55° C. for overnight followed by bleaching to obtain the decolorized samples. Chemiluminescence was reduced by the addition of glacial acetic acid. Hionic-Fluor liquid scintillation cocktail (Perkin-Elmer) was added to all samples, which were then counted with a Tri-Carb 2800TR liquid scintillation Analyzer (Perkin-Elmer).

### Treatment of MDA-MB-435 Breast Cancer Model

When palpable tumors were present in all animals (100-150 mm³), mice were randomly divided into three groups (n=8 per group). Group 1 and 2 were treated with solvent control (10% DMSO/90% normal saline) and 15 mg/kg RGD+10 mg/kg PTX mixture, respectively. Group 3 was treated with 25 mg/kg RGD2−PTX conjugates to keep the effective PTX dose at the same level as group 2 (10 mg/kg PTX motif). Each mouse was treated by i.p. injection every three days with a total of five doses. The mouse body weight and tumor volume were measured every 3 days for up to 20 days before euthanasia.

### MicroPET Imaging

Detailed procedure for positron emission tomography (PET) imaging has been reported earlier (Eur J Nucl Med. 2001;28:1326-35, which is incorporated herein by reference for the corresponding discussion). Briefly, PET scans were performed using a microPET R4 rodent model scanner (Siemens Medical Solutions). After 6 h fasting, mice were injected with about 100 μCi of 2-deoxy-2-[¹⁸F]fluoro-D-glucose (¹⁸F-FDG) or 3′-deoxy-3′-[¹⁸F]-fluorothymidine (¹⁸F-FLT) via tail vein under isoflurane anesthesia and 3-5 min PET scans were performed at 1 h postinjection (p.i.). The images were reconstructed by a two-dimensional ordered subsets expectation maximum (OSEM) algorithm with no attenuation or scatter correction. For each microPET scan, regions of interest (ROIs) were drawn over the tumor by using vendor software ASI Pro 5.2.4.0 on decay corrected whole-body coronal images. Assuming a tissue density of 1 g/mL, the ROIs were converted to MBq/g/min using a conversion factor, and then divided by the administered activity to obtain an imaging ROI-derived percent injected dose per gram (% ID/g).

Double Staining of TUNEL and Human Integrin αᵥβ₃

Frozen tissue slices (5-μm thick) were taken out from freezer and warmed for 20 min at room temperature. Fluorescent TUNEL assay was then conducted by following the manual instruction of In Situ Cell Death Detection kit (Roche, Indianapolis, Ind.). After TUNEL staining, slides were blocked with 10% goat serum in PBS for 15 min at room temperature and incubated with anti-human αᵥβ₃ antibody (MedImmune, Gaithersburg, Md.) for 1 h at room temperature. After 3×5 min washing with PBS, slides were incubated with FITC-conjugated goat anti-human secondary antibody (Jackson ImmunoResearch Laboratories, Inc., West Grove, Pa.). After staining, slides were mounted with VECTASHIELD mounting medium (Vector Laboratories, Buringame, Calif.) and examined under an epifluorescence microscope (Carl Zeiss Axiovert 200M).

### Ki67 and CD31 Immunofluorescence Staining.

Frozen tumor sections (5-μm thick) were fixed with cold acetone for 10 min and dried in the air for 30 min. After blocking with 10% donkey serum for 30 min at room temperature, the sections were incubated with rabbit anti-human Ki67 (1:100, NeoMarkers, Fremont, Calif.) or rat anti-mouse CD31 antibodies (1:100, BD Biosciences, San Jose, Calif.) separately overnight at 4° C. After incubation with Cy3-conjugated donkey anti-rabbit and FITC-conjugated donkey anti-rat secondary antibodies (1:200, Jackson ImmunoResearch Laboratories, Inc., West Grove, Pa.), the slides were mounted with DAPI-containing mounting medium and examined under an epifluorescence microscope (Carl Zeiss Axiovert 200M).

### Statistical Analysis

Statistical significance was determined by one-way ANOVA using the computer SPSS (10.0) statistic package. P value<0.05 was considered significant.

### Result

### Chemistry and Radiochemistry

The synthesis of RGD2−PTX was performed through an active ester method. PTX-SX was activated and then conjugated with the amino group of dimeric RGD peptide under a slightly basic condition. RGD2−PTX was obtained as a fluffy white powder (J Med Chem. 2005;48:1098-106, which is incorporated herein by reference for the corresponding discussion). ³H-RGD2−PTX was synthesized by the same method. However, non-radioactive PTX was added as a carrier to improve the yield. Although the specific activity of ³H-RGD2−PTX was dropped to 1.68 μCi/mg, it is still sufficient for the following biodistribution studies.

### Biodistribution of ³H-PTX and ³H-RGD2−PTX

³H-PTX and ³H-RGD2−PTX were injected at equivalent molar amount to guarantee the comparability. As seen from Table 1, Example 6, the highest concentration of ³H-PTX was found in the liver at 4 h (2389.3±408.8 ng/g). No significant difference was observed for the accumulation of ³H-PTX between the muscle (257.3±32.2 ng/g) and the tumor (239.0±56.2 ng/g). The ³H-PTX also cleared very fast from the body. As compared with the 4 h time point, the concentration of ³H-PTX at 24 h dropped by 20-fold in the liver (123.4±12.2 ng/g) and 9-fold in kidneys (38.0±13.3 ng/g). Such low levels were maintained throughout 48 h. We also observed around 3-fold decrease for the concentration of ³H-PTX in the tumor at 24 h (85.6±15.2 ng/g) as compared to that at 4 h (239.0±56.2 ng/g), which was further reduced to 45.8±1.69 ng/g (5.2-fold decrease compared with 4 h time point) at 48 h post drug administration. The tumor/muscle ratio was determined to be 0.93 at 4 h, 2.08 at 24 h, and 1.29 at 48 h.

In contrast, ³H-RGD2−PTX had a tumor uptake of 357.5±62.62 ng/g effective PTX concentration at 4 h, 229.4±50.4 ng/g at 24 h, and 148.8±40.2 ng/g at 48 h time point (Table 2, Example 6). The tumor uptake of ³H-RGD2−PTX in MDA-MB-435 tumor is significantly higher than ³H-PTX at all time points examined (P<0.001) and the tumor clearance rate is also much slower, presumably due to integrin specific delivery of PTX based on our previous experiments (J Med Chem. 2005;48:1098-106, which is incorporated herein by reference for the corresponding discussion). The muscle uptake of ³H-RGD2−PTX was also lower than ³H-PTX. The resulting tumor-to-muscle ratios of ³H-RGD2−PTX were 2.86 at 4 h, 2.82 at 24 h, and 1.74 at 48 h, which were significantly higher than those of ³H-PTX (P<0.05). It is of note that the initial liver uptake of ³H-RGD2−PTX (1252.9±109.9 ng/g at 4 h) was significantly lower than that of ³H-PTX (P<0.01). However, ³H-PTX tends to clear faster than ³H-RGD2−PTX. Renal uptake of ³H-RGD2−PTX is higher than ³H-PTX (P<0.01) at both early and late time points, which may be attributed to both renal clearance and integrin specific binding of RGD2−PTX as the endothelial cells of small glomerulus vessels of rodent kidneys express β₃ integrin. Also note that the blood activity for ³H-RGD2−PTX was considerably higher than ³H-PTX, which might be related to the metabolic instability of the construct. Overall, prominent tumor uptake and retention of RGD2−PTX may provide tumor treatment benefit over PTX.

### Treatment of MDA-MB435 Breast Cancer Model

To determine whether RGD2−PTX conjugate has better antitumor effect than the combination of PTX+RGD2 (in equal PTX dose) in vivo as we proposed, female athymic nude mice bearing MDA-MB-435 tumor were randomly divided into three groups and treated with vehicle (Saline with 10% DMSO), RGD2 (15 mg/kg) plus PTX (10 mg/kg), or RGD2−PTX conjugate 25 mg/kg (equimolar dose of PTX) every three days (a total of 5 doses). As shown in FIG. 7-2A, the combination of RGD2 plus PTX therapy started to show significant therapeutic effect as compared with the vehicle control group at day 15 when the treatment was initiated (P<0.05). However, the effectiveness of RGD2−PTX conjugate treatment became obvious as compared to the other two treatments after two doses. After day 9, RGD2−PTX conjugate group showed even more tumor suppression effect (P<0.01 compared with vehicle group, p<0.05 compared with PTX+RGD2 group). Moreover, no significant body weight difference was observed among these three treatment groups (FIG. 7-2B).

¹⁸F-FDG and ¹⁸F-FLT microPET Imaging

¹⁸F-FDG microPET is a functional imaging technique that reflects the glycolytic rate of tissues and has been used to measure the increased metabolic demand in tumor cells. Currently, the use of PET for response assessment is changing from evaluation at the end of treatment to prediction of tumor response early during the course of therapy. Therefore, we thus performed ¹⁸F-FDG microPET on day 10 after 3 doses of treatment. As shown in FIGS. 7-3A & 7-3B, the tumor uptake of ¹⁸F-FDG was decreased from 7.95±0.39% ID/g (vehicle control group) to 6.73±0.50% ID/g in PTX+RGD2 treatment group, and to 5.97±0.54% ID/g in RGD2−PTX treatment group (P<0.01). These tumor uptakes during the treatment correlated well with our therapy results at later time points. To assess the effects of therapy on tumor proliferation, ¹⁸F-FLT imaging (Cancer Res. 2007;67:1706-15, which is incorporated herein by reference for the corresponding discussion) was also conducted. No significant difference was observed among the control and two treatment groups (P>0.05, FIGS. 7-3C & 7-3D). In fact, the tumor growth curve showed a steady increase of tumor growth in all three groups, which may also suggest that the PTX could not effectively inhibit cell proliferation in this experiment.

### Immunofluorescence Staining

To evaluate whether cell apoptosis was involved in the RGD2−PTX enhanced regression on MDA-MB-435 tumors, the TUNEL assay was used to quantify cell apoptosis in tumor sections from all three groups. As shown in FIG. 7-4, vehicle-treated tumors did not show specific cell apoptosis. Combination of RGD2 with PTX for the treatment only resulted in moderately positive TUNEL staining at tumor peripheral area. In contrast, RGD2−PTX conjugate treatment group showed significant cell apoptosis throughout the tumor. At the same time, we also detected human integrin αᵥβ₃ expression on the same tissue section by immunofluorescence staining. Although TUNEL staining was quite different among these three groups, all tumor sections showed similar integrin αᵥβ₃ expression pattern. For the PTX+RGD2 treatment group, PTX seems to be accumulated only on the angiogenic edge of the tumor and cause apoptosis at the corresponding tumor periphery. The center of the tumor with necrosis and low vessel density does not allow efficient diffusion of PTX and thus little or no PTX induced apoptosis was observed. For the RGD2−PTX treatment group, TUNEL positive staining was found throughout the tumor with excellent overlay with integrin αᵥβ₃, confirming the effectiveness of integrin specific delivery of PTX.

We also carried out the CD31 staining to study the effect of PTX treatment on vascular damage. Microvessel density (MVD) analysis revealed that RGD2−PTX treated tumor had significantly lower vessel density (13.3±5.7 vessels/mm²) than the PTX+RGD2 treated tumor (24.0±3.2 vessels/mm²; P<0.01, FIG. 7-5) and solvent treated tumor (37.0±8.1 vessels/mm²; P<0.01, FIG. 7-5). The tumor vessels in PTX+RGD2 treatment group tend to have large diameters while the vessels in the RGD2−PTX treatment group tend to be small and irregular. To value whether tumor cell proliferation inhibition was also involved in the RGD2−PTX enhanced regression on MDA-MB-435 tumors, the Ki67 (cell proliferation marker) immunofluorescence was used to quantify cell proliferation in tumor sections from all groups. However, no significantly delayed cell proliferation was observed in RGD2−PTX conjugate therapy group compared with vehicle control group and combination (RGD2+PTX) group (FIG. 7-6), which was also consistent with the ¹⁸F-FLT imaging result (FIGS. 7-3C & 7-3D).

Discussion The anti-tumor efficacy of clinically used anticancer drugs is often limited by their nonspecific toxicity to proliferating normal cells, which could result in low therapeutic index and narrow therapeutic window. Previously, we have demonstrated that targeting drugs to receptors involved in tumor angiogenesis is a novel and promising approach to improve cancer treatment (J Med Chem. 2005;48:1098-106, which is incorporated herein by reference for the corresponding discussion). The RGD2−PTX was constructed from a dimeric RGD peptide E[c(RGDyK)]₂ and PTX through the 2′-hydroxy group of paclitaxel and amino group of RGD glutamate residue (J Med Chem. 2005;48:1098-106, which is incorporated herein by reference for the corresponding discussion). A metabolically unstable ester bond is preferred here, as PTX, an antimicrotubule agent, needs to be released from the RGD2−PTX construct once inside the cell in order to exert its toxicity. By targeting integrin αᵥβ₃ through the RGD motif, improved tumor specificity and cytotoxic effect of paclitaxel was observed. In this work, we evaluated the tumor therapeutic effect of RGD2−PTX in vivo in comparison with PTX+RGD2 treatment and solvent only treatment.

Although we have synthesized ¹²⁵I-RGD2−PTX and studied its distribution in vivo, the ¹²⁵I was labeled to RGD motif and the ester bond between RGD2 and PTX was metabolically unstable. Once the ester bond is broken, ¹²⁵I counting would only reflect the distribution of dimeric RGD instead of PTX. Therefore, we studied the distribution of ³H-RGD2−PTX, which is more relevant to the pharmacokinetics of PTX within RGD2−PTX. Our experimental results in vivo showed that ³H-RGD2−PTX conjugate possessed higher tumor uptake and prolonged tumor retention than ³H-PTX, which may count for the better therapeutic efficacy of RGD2−PTX than RGD2+PTX.

In the following experiments, tumor response to therapy was estimated by tumor volume measurement, ¹⁸F-FDG PET, ¹⁸F-FLT PET, and ex vivo histopathological validation. RGD2−PTX treatment showed significant tumor growth delay than the RGD2+PTX treatment group and solvent control, ¹⁸F-FDG PET also revealed reduced tumor metabolism after PTX and RGD2−PTX treatment. Ex vivo immunohistochemistry revealed that RGD2−PTX is more effective than RGD2+PTX in terms of inducing tumor apoptosis and destroying tumor vasculature. However, neither ¹⁸F-FLT PET nor Ki67 staining showed significant difference among the three treatment groups, which concurred with the observation that RGD2+PTX and RGD2−PTX slowed down the tumor growth but the tumor volume still increased with time despite of multiple dose administrations. The dose and dose interval (10 mg PTX equivalent every three days for a total of 5 doses) did not seem to cause body weight loss or other visible toxicological effect. Further studies focusing on the test of the effect of various doses and treatment frequencies are required to optimize the treatment efficacy.

Despite the successful demonstration of integrin-targeted delivery of PTX for breast cancer therapy, there are several limitations to the current study. First, although ³H-RGD2−PTX biodistribution showed higher tumor uptake and longer retention of PTX in the integrin positive MDA-MB-435 tumor than PTX, the absolute tumor uptake value is still rather low, due in part to the lipophilic character of PTX and RGD2−PTX, and small molecular sizes, leading to short circulation half-life and rapid clearance. Several strategies have been employed to increase the water-solubility and biocompatibility of paclitaxel. Notably, the commercial formulation of pacliatxel (i.e., Taxol®) contains Cremophor, which forms micelles that entrap the drug and increases blood half-life as compared to DMSO formulation used in this study. More recently, a Cremophor free, albumin stabilized formulation of paclitaxel, Abraxane®, was approved by FDA for 2^{nd}-line therapy of advanced breast cancer. We postulate that albumin-paclitaxel conjugate with RGD peptide attachment would allow both passive targeting based on the enhanced permeability and retention effect (EPR effect) of tumor vascularture and specific tumor targeting based on integrin recognition would outperform Abraxane for further enhanced anti-tumor effect of paclitaxel. Such strategy may be extended to various biocompatible nanoparticles to carry RGD peptide and PTX for controlled release therapy of cancer.

### Conclusion

We have successfully demonstrated the ability of dimeric RGD peptide to deliver paclitaxel chemotherapeutic drug to integrin positive breast cancer tumor. The treatment efficacy of RGD2−PTX was confirmed by size measurement, in vivo PET imaging and ex vivo histopathology. The tumor growth delay is related to tumor proliferation rather than tumor metabolism as confirmed by ¹⁸F-FDG/PET and ¹⁸F-FLT/PET.

### Table Legends

TABLE 1
Example 6. Tissue distribution of ³H-PTX in Balb/c nude mice
bearing MDA-MB-435 tumor. Values are mean ± SD (n = 3) and
shown as ³H-PTX concentration (ng/g Tissue).

| Organ | 4 h | 24 h | 48 h |
|---|---|---|---|
| Blood | 67.1 ± 9.8 | 42.7 ± 14.7 | 35.0 ± 1.5 |
| Skin | 135.1 ± 23.5 | 24.6 ± 3.2 | 25.4 ± 9.3 |
| Muscle | 257.3 ± 32.2 | 41.1 ± 17.2 | 35.4 ± 10.7 |
| Heart | 200.7 ± 48.5 | 37.2 ± 10.7 | 42.9 ± 10.9 |
| Lung | 329.2 ± 18.2 | 35.2 ± 5.4 | 47.9 ± 8.8 |
| Liver | 2389.3 ± 408.8 | 123.4 ± 12.2 | 132.6 ± 31.9 |
| Kidney | 339.6 ± 67.6 | 38.0 ± 13.3 | 35.7 ± 2.0 |
| Spleen | 365.5 ± 118.5 | 51.6 ± 7.6 | 36.4 ± 8.4 |
| Stomach | 180.7 ± 15.7 | 24.4 ± 4.7 | 17.2 ± 4.0 |
| Intestine | 274.1 ± 110.1 | 14.4 ± 2.5 | 12.8 ± 7.0 |
| tumor | 239.0 ± 56.2 | 85.6 ± 15.2 | 45.8 ± 1.7 |
| tumor/muscle | 0.93 | 2.08 | 1.29 |
| tumor/liver | 0.1 | 0.69 | 0.34 |
| tumor/kidney | 0.7 | 2.25 | 1.28 |

TABLE 2
Example 6. Tissue distribution of ³H-RGD2-PTX in Balb/c nude mice
bearing MDA-MB-435 tumor. Values are mean ± SD (n = 3) and
shown as ³H-RGD2-PTX concentration (ng/g Tissue).

| Organ | 4 h | 24 h | 48 h |
|---|---|---|---|
| Blood | 101.7 ± 30.8 | 143.1 ± 18.0 | 225.4 ± 12.8 |
| Skin | 144.1 ± 15.9 | 87.2 ± 16.1 | 66.0 ± 9.1 |
| Muscle | 125.1 ± 24.0 | 81.2 ± 11.4 | 85.6 ± 28.0 |
| Heart | 228.7 ± 29.8 | 170.7 ± 18.6 | 222.5 ± 16.2 |
| Lung | 300.4 ± 30.9 | 238.6 ± 75.6 | 207.8 ± 48.2 |
| Liver | 1252.9 ± 109.9 | 510.4 ± 28.9 | 545.3 ± 30.6 |
| Kidney | 1421.6 ± 289.8 | 338.9 ± 22.1 | 281.8 ± 32.6 |
| Spleen | 322.3 ± 59.3 | 228.7 ± 39.4 | 227.9 ± 28.2 |
| Stomach | 119.0 ± 16.7 | 71.63 ± 9.5 | 71.7 ± 12.2 |
| Intestine | 127.8 ± 20.3 | 52.1 ± 7.5 | 50.8 ± 9.5 |
| tumor | 357.5 ± 62.6 | 229.4 ± 50.4 | 148.8 ± 40.2 |
| tumor/muscle | 2.86 | 2.82 | 1.74 |
| tumor/liver | 0.29 | 0.45 | 0.27 |
| tumor/kidney | 0.25 | 0.68 | 0.53 |

### Example 7

### Application of Radiolabeled RGD Peptides for Myocardial Infarction Imaging Induction of Myocardial Infarction

Induction of MI was done as previously described by our laboratory. Adult female Sprague-Dawley rats (weight 150-200 g; Charles River Laboratories, Wilmington, Mass.) were used for this study. On the day of surgery, anesthesia was induced with isoflurane (5%) and the animals were intubated for mechanical ventilation. The anesthesia was then maintained with isoflurane (2%). MI was induced by ligation of the left anterior descending coronary artery 2 to 3 mm from the tip of the left auricle with a 7-0 polypropylene suture. This resulted in myocardial blanching and ST-segment elevation on an ECG monitor (Silogic EC-60 model, Silogic, Stewartstown, Pa.). In the sham operated animals, a suture was placed in the myocardium (without ligating the left coronary artery).

Assessment of Left Ventricular Contractility with Echocardiography

To assess cardiac function, rats received isofluorane (2%) for general anesthesia and were placed on the scanning table. Echocardiographic images were obtained using a dedicated small animal high-resolution-imaging unit and a 30-MHz linear transducer (Vevo 770®, Visualsonics, Toronto, Canada). Using the parasternal short axis view, left ventricular end-diastolic and end-systolic diameters (LVEDD and LVESD, respectively) were measured, and left ventricular fractional shortening was calculated as (LVEDD-LVESD)/LVEDD*100.

### MicroPET Scanning

Animals were anesthetized with isofluorane (2%) and injected with approximately 1 mCi (37 MBq) of ⁶⁴Cu-DOTA-E{E[c(RGDyK)]₂}₂ (or ¹⁸F-FPRGD2) via the tail vein and allowed to recover. To determine the best signal/background ratio, animals were scanned at 1 h after injection of the tracer. At the time of scanning, animals were anesthesized with isofluorane (2%) and prone positioned on the microPET Concorde R4 rodent model scanning gantry (Siemens A G, Malvern, Pa.). The scanner has a computer-controlled bed and 10.8-cm transaxial and 8-cm axial fields of view (FOV). Pixel size was of 0.845×0.845×1.2 mm, and a slice thickness of 0.845 mm and full width half maximum of 1.66, 1.65 and 1.84 mm for tangential, radial, and axial orientation, respectively. It has no septa and operates exclusively in the 3-dimensional list mode. A 15 minute static acquisition was performed with the mid thorax in the center of the field of view (FOV), and images reconstructed using a filtered back projection algorithm.

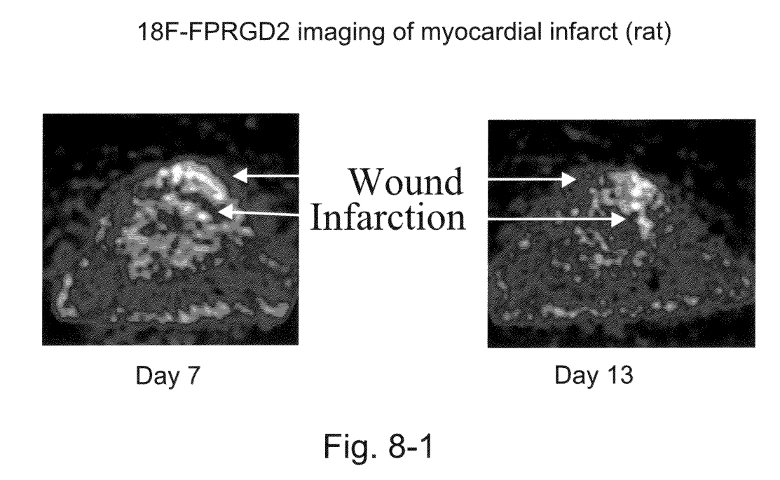

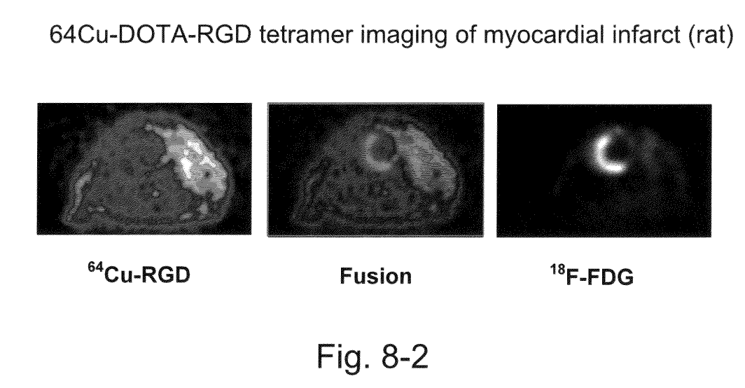

FIG. 8-1 illustrates microPET images of rat myocardial infarction with 18F-FPRGD2. Transaxial images of the same animal on day 7 and 13 were shown. Both wound and the iinfarcted myocardium showed positive signal.

FIG. 8-1: At day 7 postoperatively, sham operated animals did not have significant myocardial uptake of ¹⁸F-FPRGD2 (data not shown). MI induction was associated with a significant increase in uptake of ¹⁸F-FPRGD2 in the anterolateral wall of the myocardium. Such signal remained high at day 13, and then decreased over time until it reached baseline levels at day 24. Importantly, the tracer uptake was only seen in the areas supplied by the ligated coronary artery, and not in remote areas.

FIG. 8-2 illustrates microPET images of rat myocardial infarction with 64Cu-DOTA-RGD tetramer and FDG. In particular, the representative images are the following: ⁶⁴Cu-DOTA-RGD tetramer (left), ¹⁸F-FDG (right), and ⁶⁴Cu-DOTA-RGD tetramer-¹⁸F-FDG fused image (middle). FDG scan shows that coronary artery ligation resulted in a lack of ¹⁸F-FDG uptake, and that the uptake of ⁶⁴Cu-DOTA-RGD tetramer occurs in areas supplied by the ligated coronary artery. Fusion of both scans results in complementation of ¹⁸F-FDG and ⁶⁴Cu-DOTA-RGD tetramer signals. There is also increased uptake in the area of the surgical wound.

In FIG. 8-2: At Day 3 after induction of MI, the animals were scanned with ⁶⁴Cu-DOTA-E{E[c(RGDyK)]₂}₂ (1 h postinjection) and then re-injected with ¹⁸F-FDG (for assessment of myocardial viability). ¹⁸F-FDG and ⁶⁴Cu-DOTA-VEGF₁₂₁ images were fused showing that the ⁶⁴Cu-DOTA-VEGF₁₂₁ myocardial signal matched extremely well to areas of infarcted myocardium as evidenced by a lack of ¹⁸F-FDG uptake. On the other hand, in sham-operated animals, there were no infarcted areas, and thus no lack of ¹⁸F-FDG uptake (data not shown). Furthermore, post operatively, animals (both sham and Ml groups) had increased uptake of ¹⁸F-FDG and ⁶⁴Cu-DOTA-VEGF₁₂₁ at the level of the surgical wound, consistent with an inflammatory response.

### Example 8

Stroke Imaging with 18F-FPRGD2
Induction of dMACO Stroke Model

Anesthesia for Sprague-Dawley rats (290-350 g) was induced by 5% isoflurane and maintained by 2-3% isoflurane. A ventral midline incision was made and the two CCAs were isolated. Snares were placed around the CCAs and the animal was placed on its right side. A 2 cm vertical scalp incision was made midway between the left eye and ear. The temporalis muscle was bisected and a 2 mm burr hole was made at the junction of the zygomatic arch and squamous bone. The distal MCA was exposed and ligated above the rhinal fissure with a 10-0 suture. The CCA snares were tightened to occlude the CCAs for 2 h. In the permanent MCA occlusion model, both CCAs were then released, while the distal MCA remained occluded.

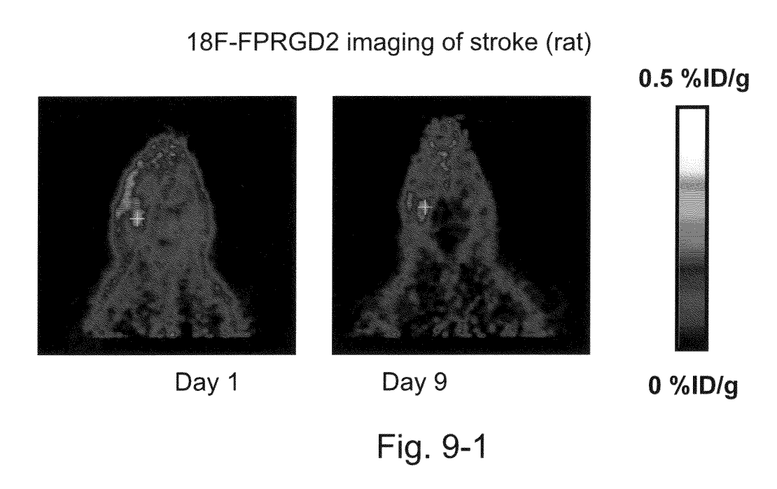

FIG. 9-1 illustrates representative coronal images of microPET scans of stroke rats at day 1 and day 9 after a suture model produced by permanent occlusion of the distal middle cerebral artery (dMCAO). Both wound and the lesion were detectable at day 1. At day 9, the wound signal is significantly decreased, but the signal in the lesion reflecting angiogenesis is remained.

FIG. 9-1: Representative coronal images of microPET scans of stroke rats at day 1 and day 9 after a suture model produced by permanent occlusion of the distal middle cerebral artery (dMCAO). Both wound and the lesion were detectable at day 1. At day 9, the wound signal is significantly decreased but the signal in the lesion reflecting angiogenesis is remained.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

1. A RGD compound comprising:
a multimeric RGD (arginine-glycine-aspartic acid) peptide;
a tag, wherein the tag is selected from a detecting unit, a therapeutic unit, or a combination thereof; and
a linker connecting the tag and multimeric RGD peptide.

2. The RGD compound of claim 1, wherein the multimeric RGD peptide can include 2 or more RGD peptide units.

3. The RGD compound of claim 1, wherein the multimeric RGD peptide is selected from: an RGD dimer peptide (E[c(RGDyK)]₂), an RGD tetramer peptide (E{E[c(RGDyK)]₂}₂), or an RGD octamer peptide (E{E{E[c(RGDyK)]₂}₂}₂).

4. The RGD compound of claim 1, further comprising a second tag, where the second tag is selected from a detecting unit, a therapeutic unit, or a combination thereof, and wherein the tag and the second tag are not the same.

5. The RGD compound of claim 1, wherein the RGD peptide unit is a cyclic peptide containing the Arg-Gly-Asp amino acid sequence.

6. The RGD compound of claim 2, wherein the cyclic peptide is selected from a head-to-tail cyclized peptide or a cyclized peptide via a disulfide bond.

7. The RGD compound of claim 1, wherein the linker is selected from:
a carbohydrate, a peptide, a polyethylene glycol (PEG), or a combination thereof.

8. The RGD compound of claim 7, wherein linker is a poly(ethylene glycol) having a molecular weight of about 200 to 20,000.

9. The RGD compound of claim 1, wherein the tag is a radiolabel selected from ¹⁸F, ^{76/77}Br, ^{123/124/125/131}I, or ²¹¹At.

10. The RGD compound of claim 1, wherein the tag is a 4-fluorobenzoyl group.

11. The RGD compound of claim 1, wherein the tag is a macrocyclic chelating agent that is chelated with a metal.

12. The RGD compound of claim 1, wherein the macrocyclic chelating agent is 1,4,7,10-tetraazadodecane-N,N′,N″,N′″-tetraacetic acid (DOTA) and the metal is ⁶⁴Cu.

13. The RGD compound of claim 1, wherein the macrocyclic chelating agent is 1,4,7-triazacyclononane-1,4,7-triacetic acid (NOTA) and the metal is ⁶⁸Ga.

14. The RGD compound of claim 1, wherein the macrocyclic chelating agent is 6-hydrazinonicotinic (HYNIC) and the metal is ^{99m}Tc.

15. The RGD compound of claim 1, wherein the tag is a macrocylic chelating agent complexed with a radiolabel, wherein the macrocyclic chelating agent is selected from: 1,4,7,10-tetraazadodecane-N,N′,N″,N′″-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), diethylenetriaminepentaacetic (DTPA), 4,11-bis(carboxymethyl)-1,4,8,11-tetraazabicyclo[6.6.2]hexadecane hexaazamacrocyclic cage ligand (CB-TE2A), 1-N-(4-aminobenzyl)-3,6,10,13,16,19-hexaazabicyclo[6.6.6]eicosane-1,8-diamine (SarAr), 6-hydrazinonicotinic (HYNIC), diamide dithiolate ligand system (N2S2), or mercaptoacetyl-triglycine (MAG3), wherein the radiolabel is selected from: ⁶⁰Cu, ⁶¹Cu, ⁶²Cu, ⁶⁴Cu, ⁶⁷CU, ⁶⁷ Ga, ⁶⁸Ga, ⁸⁶Y, ⁸⁸Y, ⁹⁰Y, ¹⁷⁷Lu, ²¹²Bi, ²¹³Bi, ¹⁵³Gd, ¹⁴⁹Tb, ¹⁶¹Tb, ¹⁵⁷Dy, ¹⁶⁵Dy, ¹⁶⁵Er, ¹⁶⁹Er, ¹⁷¹ Er, ¹⁶⁷Tm, ¹⁶⁹Yb, ¹⁵³Sm, ¹⁶⁶Ho, ¹¹¹In, ^{94m}Tc, or ^{99m}Tc.

16. The RGD compound of claim 1, wherein the tag is a chemotherapeutic selected from: paclitaxel, doxorubicin, methotrexate, chlorambucil, or 5-fluorodeoxyuridine.

17. The RGD compound of claim 1, having structure A as shown in FIG. 1-5a.

18. The RGD compound of claim 1, having structure B as shown in FIG. 1-5b.

19. The RGD compound of claim 1, having structure C as shown in FIG. 1-5c.

20. The RGD compound of claim 1, having structure D as shown in FIG. 1-5d.

21. The RGD compound of claim 1, having structure E as shown in FIG. 1-5e.

22. The RGD compound of claim 1, having structure F as shown in FIG. 1-6b.

23. A kit, comprising a RGD compound of claim 1 and directions for use.

24. A method of imaging tissue, cells, or a host comprising:
contacting with or administering to a tissue, cells, or host an RGD compound of claim 1, and
imaging the tissue, cells, or host, with an imaging system.

25. The method of claim 24, wherein the imaging is performed in vivo or in vitro.

26. The method of claim 24, wherein imaging includes imaging cancer in the tissue, cells, or host.

27. The method of claim 24, wherein imaging includes imaging an infarct in the tissue, cells, or host.

28. The method of claim 24, wherein imaging includes imaging a stroke in the tissue, cells, or host.

29. The method of claim 24, wherein the imaging system is a PET imaging system.

30. A method of diagnosing the presence of one or more angiogenesis related diseases or related biological events in the tissue, cells, or a host comprising:
contacting or administering to a tissue, cells, or a host an RGD compound of claim 1; and
imaging the tissue, cells, or a host with an imaging system, wherein the location of the RGD compound corresponds to the location of the angiogenesis related diseases or related biological events.
